SEQUENCE LISTING (19 sequences)

SEQ ID NO: 1 - protein, 180 aa, unidentified
MAPGVARGPTPYWRLRLGGAALLLLLIPVAAAQEPPGAACSQNTNKTCEECLKNVSCLWCNTNKACLDYPVTSVLPPASLCKLSSARWGVCWVNFEALIITMSVVGGTLLLGIAICCCCCCRRKRSRKPDRSEEKAMREREERRIRQEERRAEMKTRHDEIRKKYGLFKEENPYARFENN

SEQ ID NO: 2 - protein, 180 aa, synthetic construct
MAPGVARGPTPYWRLRLGGAALLLLLIPVAAAQEPPGAACSQNTNKTCEECLKNVSCLWCNTNKACLDYPVTSVLPPASLCKLSSARWGVCWVNFEALIITMSVVGGTLLLGIAICCCCCCRRKRSRKPDRSEEKAMREREERRIRQEERRAEMKTRHDEIRKKAGLFKEENPAARFENN

SEQ ID NO: 3 - protein, 145 aa, synthetic construct
MAPGVARGPTPYWRLRLGGAALLLLLIPVAAAQEPPGAACSQNTNKTCEECLKNVSCLWCNTNKACLDYPVTSVLPPASLCKLSSARWGVCWVNFEALIITMSVVGGTLLLGIAICCCCCCRRKRSRKPYGLFKEENPYARFENN

SEQ ID NO: 4 - protein, 237 aa, synthetic construct
MAPGVARGPTPYWRLRLGGAALLLLLIPVAAAQEPPGAACSQNTNKTCEECLKNVSCLWCNTNKACLDYPVTSVLPPASLCKLSSARWGVCWVNFEALIITMSVVGGTLLLGIAICCCCCCRRKRSRKPDRSEEKAMREREERRIRQEERRAEMKTRHDEIRKKYGLFKEENPYARFENNDPPVATDRSEEKAMREREERRIRQEERRAEMKTRHDEIRKKYGLFKEENPYARFENN

SEQ ID NO: 5 - protein, 51 aa, unidentified
DRSEEKAMREREERRIRQEERRAEMKTRHDEIRKKYGLFKEENPYARFENN

SEQ ID NO: 6 - protein, 6 aa, synthetic construct
DPPVAT

SEQ ID NO: 7 - protein, 5 aa, synthetic construct
GGGGS

SEQ ID NO: 8 - protein, 10 aa, synthetic construct
GGGGSGGGGS

SEQ ID NO: 9 - protein, 20 aa, synthetic construct
GGGGSGGGGSGGGGSGGGGS

SEQ ID NO: 10 - protein, 6 aa, synthetic construct
GGGGGG

SEQ ID NO: 11 - protein, 8 aa, synthetic construct
GGGGGGGG

SEQ ID NO: 12 - protein, 5 aa, synthetic construct
EAAAK

SEQ ID NO: 13 - protein, 10 aa, synthetic construct
EAAAKEAAAK

SEQ ID NO: 14 - protein, 15 aa, synthetic construct
EAAAKEAAAKEAAAK

SEQ ID NO: 15 - protein, 46 aa, synthetic construct
AEAAAKEAAAKEAAAKEAAAKALEAEAAAKEAAAKEAAAKEAAAKA

SEQ ID NO: 16 - protein, 5 aa, unidentified
VVVHN

SEQ ID NO: 17 - protein, 6 aa, synthetic construct
VVVHNG

SEQ ID NO: 18 - protein, 6 aa, synthetic construct
VVVHNC

SEQ ID NO: 19 - protein, 8 aa, unidentified
ILXCXXDE

(19) United States
(12) Patent Application Publication
(10) Pub. No.: US 2026/0034239 A1
(43) Pub. Date: Feb. 5, 2026

(54) LOADED EXTRACELLULAR VESICLE

(71) Applicant: Oxford University Innovation Limited, Oxford (GB)

(72) Inventors: Carla MARTIN, Oxford (Oxfordshire) (GB); Thomas ROBERTS, Oxford (Oxfordshire) (GB); Matthew WOOD, Oxford (Oxfordshire) (GB)

(21) Appl. No.: 18/996,093

(22) PCT Filed: Jul. 25, 2023
(86) PCT No.: PCT/GB2023/051958

§ 371 Date: Jan. 17, 2025

(30) Foreign Application Priority Data
Jul. 25, 2022 (GB) 2210861.7

(51) Int. Cl. A61K 47/69; A61K 9/50; A61K 38/46; A61K 47/65; A61P 35/00; C12N 5/071; C12N 9/22; C12N 15/11; C12N 15/88

(52) U.S. Cl. CPC A61K 47/6901; A61K 9/5068; A61K 38/465; A61K 47/65; A61P 35/00; C12N 5/0686; C12N 9/226; C12N 15/11; C12N 15/88; C12N 2310/20

(57) ABSTRACT

The present invention relates to compositions comprising an extracellular vesicle (EV). In particular, the extracellular vesicle (EV) comprises a single pass EV transmembrane protein fused to a moiety on the surface of the EV and/or a cargo molecule. The composition may be used to deliver the cargo molecule. Methods of manufacturing the composition are also provided.

### FIELD OF THE INVENTION

The present invention relates to compositions comprising an extracellular vesicle (EV). In particular, the extracellular vesicle (EV) comprises a single pass EV transmembrane protein fused to a moiety on the surface of the EV and/or a cargo molecule. The composition may be used to deliver the cargo molecule. Methods of manufacturing the composition are also provided.

### BACKGROUND TO THE INVENTION

Extracellular vesicles (EVs) are cell-derived, membranous nanoparticles released by cells to the extracellular environment that facilitate the intercellular transfer of proteins and nucleic acids¹. EVs are heterogeneous in terms of size, molecular content, and biogenesis. Microvesicles are formed by direct outward budding of the plasma membrane, while exosomes derive from intraluminal vesicles (ILVs) formed by inward budding of the limiting membrane of late endosomal multivesicular bodies (MVBs). Upon fusion of MVBs with the plasma membrane, released ILVs are then known as exosomes.² While exosomes size ranges between 50-150 nm, microvesicles can be as large as 1,000 nm¹. EVs can be harnessed for pharmaceutical purposes by modifying them to include therapeutically relevant cargo molecules. EVs offer significant advantages over other delivery strategies such as viral delivery, that is hampered by anti-virus immunity³. In contrast, EVs are autologous, physiological entities with the potential for repeat dosing. Furthermore, given that EVs are fully biodegradable, toxicity resulting from an accumulation of nanoparticles due to repeated doses should be avoided. The enrichment of specific proteins in EVs relative to expression levels in their respective producer cell suggests that there are mechanisms that regulate the sorting of protein cargo to EVs⁴. Although these mechanisms are beginning to be deciphered, they still are not fully understood. Post-translational modifications (PTMs) such as myristylation, ubiquitination, and ubiquitin-like modifications have been shown to play a role in EV-protein sorting⁵.

Loading of therapeutic cargo molecules constitutes one of the main challenges for EV-based therapy. Not only must this cargo be present in the EVs, but it also ideally and/or in certain situations the cargo needs to be releasable from the EV, such that it is able to escape the endolysomal system so that it can reach the desired intracellular location once taken up by the recipient cells. Current methods for endogenous EV-protein loading often rely on multipass transmembrane proteins, particularly tetraspanins such as CD63⁶⁻⁹, that despite showing a high loading capacity are not generally amenable to further engineering on their extracellular domains (i.e. addition of targeting peptides¹⁰) given that their C- and N-termini are both situated on the luminal side. There is therefore a need for more suitable EV scaffold proteins that allow for both highly efficient intracellular EV-loading and that can be modified with targeting moieties at the EV surface.

### SUMMARY OF THE INVENTION

The project leading to this application has received funding from the European Union's Horizon 2020 research and innovation programme under grant agreement 721058.

The inventors have successfully provided an EV comprising a single pass EV transmembrane protein fused to a moiety on the surface of the EV and/or a cargo molecule.

In particular, the inventors made use of a bioinformatics approach to identify proteins having a surprising utility in efficiently loading an EV with cargo and enabling delivery of the cargo, for example in vitro and/or in vivo. Advantageously, the efficiency of loading and delivery relative to existing proteins used for loading cargo to EVs, such as CD63 and/or Lamp2B, is drastically improved. Therefore, the present invention allows for significantly improved delivery of cargo using EVs, for example in a therapeutic setting.

In particular, the present invention has all the advantages associated with the use of single-pass transmembrane EV proteins for EV engineering (i.e. ease of engineering, in particular for surface display) without the disadvantages associated with known single-pass transmembrane EV proteins, such as Lamp2B. In particular, as compared to Lamp2B, the single-pass transmembrane EV protein of the present invention allows for increased loading of fused moieties and/or cargoes. The single-pass transmembrane EV protein of the present invention also allows for EVs that co-express one or more additional exogenous constructs, alongside engineered PTTG1IP, including additional scaffold proteins comprising second and subsequent cargos as well as endosomal escape moieties such as VSVG. PTTG1IP is particularly preferable to Lamp2B wherein the scaffold is being N-terminally engineered (i.e. to allow for a cargo or moity to be displayed in the EV surface).

In accordance the invention provides the following aspect:
[1] a composition comprising an extracellular vesicle (EV), wherein the EV comprises a single pass EV transmembrane protein fused to:
   (a) a moiety on the surface of the EV, and/or
   (b) a cargo molecule;
[2] the composition according to [1], wherein the single pass EV transmembrane protein has a maximum molecular weight of 50 kDa;
[3] the composition according to [1] or [2], wherein the single pass EV transmembrane protein comprises one or more post translational modification optionally selected from one or more of glycosylation such as N-linked GlcNAc asparagine, prenylation, ubiquitination, myristoylation, sumoylation, phosphorylation such as kinase specific phosphorylation, lipidation, hydroxyproline, pyrolidone and carboxylic acid;
[4] the composition according to any one of [1]-[3], wherein the composition is substantially devoid of vesicle aggregates; and/or the diameter of the EV is 30 to 150 nm or 150 to 1000 nm;
[5] the composition according to any one of [1]-[4], wherein the single pass EV transmembrane protein comprises:
   (a) a polypeptide sequence having at least 80%, at least 90%, at least 95% or at least 100% sequence identity to SEQ ID NO: 1
   (b) a polypeptide sequence having at least 80%, at least 90%, at least 95% or at least 100% sequence identity to any one of SEQ ID NOs: 2 to 5;
   (c) at least 10, at least 20, at least 30, at least 40 or at least 50 contiguous amino acid residues from the polypeptide sequence of any one of SEQ ID NOs: 1 to 5; and/or
   (d) pituitary tumour-transforming gene 1 interacting protein (PTTG1IP), or a variant or fragment thereof, optionally wherein the variant or fragment thereof retains the intrinsic cleavage activity of PTTG1IP;
preferably wherein the EV comprises a further exogenous protein, more preferably wherein the further exogenous protein is endosomal escape moiety, and most preferably wherein the endosomal escape moiety is VSVG;
[6] the composition according to any one of [1]-[5], wherein the moiety on the surface of the EV is selected from one or more of a peptide and/or protein, a targeting peptide and/or targeting protein which binds to a molecule present on a cell to be targeted, a therapeutic moiety such as a receptor decoy, an endosomal escape moiety, an enzyme, a nuclease, a CRISPR-associated protein (Cas) such as SaCas9, SpCas9, Cas9, Cas12, Cas13 and variants and fusions thereof, a Transcription activator-like effector nucleases (TALEN) and variants and fusions thereof, a meganuclease and variants and fusions thereof, a zinc finger nuclease and variants and fusions thereof, an antibody and/or antigen-binding variant or fragment thereof, a single chain variable fragment (scFv), VHH, a nanobody, a nucleic acid-binding protein and/or peptide, a RNA- and/or DNA-binding protein, viral-binding protein, a small molecule drug, a nucleic acid, a nucleic acid analogue, an unnatural nucleic acid, gRNA, miRNA, shRNA, siRNA, piRNA, PMO, DNA and a DNA plasmid encoding a therapeutic peptide and/or protein; optionally wherein
   (a) the moiety on the surface of the EV is a targeting peptide and/or targeting protein which binds to a molecule present on a cell to be targeted, optionally wherein the targeting peptide and/or targeting protein binds to a molecule present on a cell of the liver, preferably a hepatocyte; the heart, preferably a cardiomyocytes or a smooth muscle cell; the brain or the nervous system, preferable a neurone or a glial cell, most preferably a sensory neurone a motor neurone or an interneuron; and/or
   (b) the moiety on the surface of the EV is selected from a peptide binding protein or an RNA binding protein, optionally wherein the peptide binding protein is capable of binding a Cas protein, preferably Cas9 or Cas12, or an adeno-associated virus (AAV), preferably an AAV capsid, and most preferably an AAV8 or AAV9 capsid;
[7] the composition according to any one of [1]-[6], wherein the cargo molecule is:
   (a) inside or outside the EV and/or
   (b) is an exogenous cargo molecule.
[8] the composition according to any one of [1]-[7], wherein the cargo molecule is selected from one or more of a therapeutic cargo, a peptide and/or protein, an enzyme, a nuclease, a CRISPR-associated protein (Cas) such as SaCas9, SpCas9, Cas9, Cas12, Cas13 and variants and fusions thereof, a Transcription activator-like effector nucleases (TALEN) and variants and fusions thereof, a meganuclease and variants and fusions thereof, a zinc finger nuclease and variants and fusions thereof, an antibody and/or antigen-binding variant or fragment thereof, a single chain variable fragment (scFv), VHH, a nanobody, a nucleic acid-binding protein and/or peptide, a RNA- and/or DNA-binding protein, viral-binding protein, a small molecule drug, a nucleic acid, a nucleic acid analogue, an unnatural nucleic acid, gRNA, miRNA, shRNA, siRNA, piRNA, PMO, DNA and a DNA plasmid encoding a therapeutic peptide and/or protein;
   optionally wherein the cargo molecule is a guide RNA, a Cas protein, preferably Cas9 or Cas12, or an adeno-associated virus (AAV) and/or wherein the therapeutic cargo is luminally loaded;
[9] the composition according to any one of [1]-[8], wherein the composition further comprises a release system, preferably wherein the release system comprises:
   (a) an organic compound-based release system;
   (b) a polypeptide-based release system;
   (c) a cis-cleaving polypeptide-based release system;
   (d) a cis-cleaving polypeptide-based release system comprising an intein;
   (e) a chemically induced dimerization system; and/or
   (f) a reversible protein-protein interaction module;
[10] the composition according to [9], wherein the release system can be activated to release the cargo molecule from the EV;
[11] the composition according to any one of [1]-[10], wherein the composition is co-administered with, and/or further comprises, an endosomal escape moiety that enhances release of the EV from endosomes;
[12] the composition according to [11], wherein the molecule that enhances release of the EV from endosomes is:
   (a) a molecule that binds to protons;
   (b) a fusogenic protein; or
   (c) a cationic peptide or polymer;
[13] the composition according to [11] or [12], wherein the molecule that enhances release of the EV from endosomes is:
   (a) an endosmolytic compound;
   (b) chloroquine, or a variant or derivative thereof, such as a proton binding variant or derivative thereof; or
   (c) a cell penetrating peptide (CPP);
[14] the composition according to any one of [1]-[13], wherein the EV is an exosome;
[15] the composition according to any one of [1]-[14], wherein the EV is derived from HEK293 cells, HEK293T cells, adipocytes, and/or mesenchymal stem cells;
[16] the composition according to any one of [1]-[15] for use in a method of delivering the cargo molecule in vitro, in vivo, and/or in situ;
[17] the composition according to any one of [1]-[16] and at least one pharmaceutically acceptable excipient, for use in a method of therapy in a subject;
[18] the composition according to any one of [1]-[17] and at least one pharmaceutically acceptable excipient, for use in a method of treating and/or preventing Alzheimer's disease, autoimmune conditions, cancer, cardiovascular disease, cystic fibrosis, Duchenne muscular dystrophy, haemophilia, Huntington's disease, lysosomal storage disease, liver disorders, macular degeneration, myotonic dystrophy, neuromuscular disease, Parkinson's disease, sepsis, spinal muscular atrophy, stroke, genetic disorders, CNS conditions, neuro-degenerative disorders, heart disorders, Phenylketonuria, heart failure or ALS;
[19] the composition according to [17] or [18], wherein said use is gene therapy and/or gene-editing;
[20] the use of a composition according to any one of [1]-[15] for purifying an EV;
[21] the in vitro or ex vivo use of a composition according to any one of [1]-[15] as a research tool, a diagnostic tool, an imaging tool, a biological reference material, an experimental control and/or an experimental standard;
[22] the use according to [20] or [21], wherein the composition is immobilised to a solid support;
[23] a method of manufacturing a composition according to any one of [1]-[15]comprising:
   (a) introducing into a host cell a polynucleotide construct encoding the single pass EV transmembrane protein;
   (b) expressing the polynucleotide construct in the host cell; and
   (c) obtaining EVs in which the single pass EV transmembrane protein has been expressed;
[24] the method according to [23], wherein the host cell is cultured in a bioreactor to produce a conditioned media and the EVs are obtained from the conditioned media;
   optionally wherein the EVs are purified from the conditioned media using one or more of liquid chromatography, tangential flow filtration, ultracentrifugation, density-gradient separation, antibody precipitation and polymer precipitation; and
[25] a polypeptide construct comprising a single pass EV transmembrane protein fused to:
(a) a moiety on the surface of the EV; and/or (b) a cargo molecule, according to any one of [1]-[10]; a polynucleotide construct encoding the polypeptide construct; or a cell comprising the polypeptide construct and/or the polynucleotide construct.

### DESCRIPTION OF THE FIGURES

FIG. 1 In silico identified EV-enriched PTM annotations.

(a) Venn diagram of statistically significant EV-enriched PTMs in HEK293T, MSCs and adipocytes. Numbers in the diagram refer to the number of PTMs identified for each group. Violin plots representing the EV to cell abundance ratios (log₁₀) of proteins with statistically significant EV-enriched PTM annotations identified in HEK293T (b). Only EV-enriched PTM annotations common across all three cell lines are shown. Numbers on the y-axis depict the number of proteins with the given PTM in the dataset. Violin plot of the EV to cell abundance ratios (log₁₀) of all proteins in the dataset is also shown. Median value of all proteins in the dataset is represented as a dashed line. *P<0.05.

FIG. 2 N-glycosylation promotes PTTG1IP EV-sorting.

(a) Scheme of PTTG1IP N-glycosylation mutants fused to GFP. (b) Western blot of WT PTTG1IP and NGlcNAc mutants fused to GFP in HEK293T cells. Cell lysates were treated with PNGase F to remove all N-glycosylation or with Endonuclease H (EndoH) to remove high-mannose and hybrid N-glycosylations. Lysates were blotted against ACTB (beta-actin) and GFP. (c) Western blot of WT PTTG1IP and NGlcNAc mutants fused to GFP in HEK293T EVs and cells. Cell lysates and isolated EVs were blotted against ACTB (beta-actin) and ALIX respectively. Both cell lysates and isolated EVs were blotted against GFP. (d) Percentage of GFP+ EVs quantified by single-vesicle analysis of conditioned media from HEK293T cells transfected with GFP-fusion constructs. (e) Median fluorescence intensity of GFP+ EVs. Data are presented as mean+SEM, n=3 replicates, *P<0.05, **P<0.01, ***P<0.001.

FIG. 3 EV-loading assessment of PTTG1IP-engineered variants.

(a) PTTG1IP protein structure and length. (b) Scheme of PTTG1IP variants fused to GFP. PTTG1IP-2YA has its two YXXL endocytosis motifs mutated to AXXL and PTTG1IP-130-164Del has a deletion from amino acid 130 to 164. (c) Percentage of GFP+ EVs quantified by single-vesicle analysis of conditioned media from HEK293T cells transfected with GFP-fusion constructs. (e) Median fluorescence intensity of GFP+ EVs. Data are presented as mean+SEM, n=3 replicates, *P<0.05.

FIG. 4 EV-mediated delivery of Cre to reporter cells by PTTG1IP-engineered variants.

(a) Scheme of tested Cre fusion constructs. SC is a self-cleaving endopeptidase derived from Shewanella oneidensis. The intein is a self-cleaving sequence from Myobacterium tuberculosis. PTTG1IPDel has a deletion from amino acid 130 to 164. PTTG1IP-130-180aa has amino acids 130-180 duplicated on PTTG1IP C-terminus. (b) Timeline of EV-transfer experiment to Cre reporter cells. 24h after donor cell transfection with Cre constructs and VSVG, media is replaced with fresh OptiMEM and EVs are isolated 48h later by size exclusion liquid chromatography (SEC). Isolated EVs are then transferred to recipient cells in 96-well plates (5x 109 EVs/ml). 24h later media is replaced with DMEM 10% FBS and after 24h cells are analysed for GFP expression by flow cytometry. (c) Scheme of the Cre reporter cells. RFP is constitutively expressed by reporter cells while GFP is only expressed when the floxed stop cassette is excised by Cre. (d) GFP positive cells quantified by flow cytometry 48h after EV-transfer to T47D cells. (e) GFP positive cells quantified by flow cytometry 48h after EV-transfer to HeLa cells. (f) EV-transfer dose response in B16 cells. (g) EV-transfer dose response in T47D cells. Data are presented as mean+SEM, n=3 replicates, ***P<0.001, ****P<0.0001. FIG. 5 EV-mediated delivery of Cre to reporter mice.

(a) Timeline of EV-transfer experiment to Cre reporter mice. 24h after donor cell transfection with Cre constructs and VSVG, media is replaced with fresh OptiMEM and EVs are isolated 48h later by SEC. 10 days before EV transfer, mice were injected with 500,000 B16F10 Cre reporter cells/mouse. On day 10, isolated EVs were injected to recipient mice by intra-tumoural injection (1×10¹⁰ EVs/mice). 4 days later, tumours are harvested and Cre activity is analysed. (b) Cre recombination activity in mice tumours treated with PTTG1IP-intein-Cre+VSVG, Cre+VSVG or VSVG only (control EVs). Cre recombination site was amplified by PCR and the products resolved by agarose gel electrophoresis. Cre recombination is indicated by a shorter 900 bp product (arrow) while non-recombined site is observed as a 1,700 bp product. Recombination percentages are indicated for recombined samples

FIG. 6 GFP expression in mice tumours.

GFP expression in mice tumours injected intratumourally with PTTGITP-intein-Cre+VSVG EVs, Cre+VSVG EVs or PBS examined by ICH. n=3 mice.

FIG. 7 EV-mediated delivery of Cas9 to reporter cells.

(a) Scheme of the Cas9 stoplight reporter construct. mCherry is constitutively expressed under a CMV promoter, followed by an F2A self-cleaving peptide sequence, a Cas9-binding linker region and a stop codon. Two GFP open reading frames that are frameshifted by one or two nucleotides are placed after the stop codon, separated by a F2A sequence. Upon Cas9 cleavage and indel formation, the translation reading frame will be shifted (on 2/3 of the editing events) and GFP will be expressed together with mCherry. (b) Timeline of EV-transfer experiment to Cas9 reporter cells. 24h after donor cell transfection with Cas9 constructs and VSVG, media is replaced with fresh OptiMEM and EVs are isolated 48h later by SEC. Isolated EVs are then transferred to recipient cells (that were transfected with sgRNA 24h before) in 96-well plates (3×10⁹ EVs/ml). 24h later media is replaced with DMEM 10% FBS and after 24h cells are analysed for GFP expression by flow cytometry. (c) GFP positive cells quantified by flow cytometry 48h after EV-transfer to sgRNA-transfected reporter cells. (d) Timeline of EV-transfer experiment to reporter cells. In this case, reporter cells did not express sgRNA. (e) GFP positive cells quantified by flow cytometry 48h after EV-transfer to reporter cells. Data are presented as mean+SEM, n=3 replicates, **P<0.01, ***P<0.001.

FIG. 8 Bioinformatics analysis scheme.

Protein motif, domain, and post-translational modification (PTM) data was extracted from the UniProt database. Motif and PTM data were then manually cured to remove duplicates and pool together identical features. In the case of PTMs, additional groups of related PTMs were pooled and included in the analysis. Protein features data was integrated with mass spectrometry proteomics data²⁴ (from HEK293T, MSCs, and adipocytes) and only features that appear on at least 5 proteins were included in the analysis due to statistical requirements. Distributions of proteins with a given feature and that of all proteins was then compared by Kruskall-Wallis and tested for significance (P<0.05). Kolmogorov-Smirnov test followed by Benjamini-Yekuteli post hoc test is then applied to test for features that are significantly EV-enriched (P<0.05). The distribution of EV to cell abundance ratio (log₁₀) of proteins with significantly EV-enriched features was then represented and relevant features were manually selected.

FIGS. 9 and 10 In silico-identified EV-enriched PTM annotations in MSCs and adipocytes.

Violin plots representing the EV to cell abundance ratios (log₁₀) of proteins with statistically significant EV-enriched PTMs identified in MSCs (FIG. 9) and adipocytes (FIG. 10). Only EV-enriched annotated PTMs common across all three cell lines are shown. Numbers on the y-axes depict the number of proteins with the given PTM in the dataset. Violin plot of the EV to cell abundance ratios (log₁₀) of all proteins in the dataset is included as a reference. Median value of all proteins in the dataset is represented as a dashed line. *P<0.05.

FIG. 11 In silico-identified EV-enriched annotated motifs.

Violin plots representing the EV to cell abundance ratios (log₁₀) of proteins with statistically significant EV-enriched annotated motifs identified in HEK293T (a), MSCs (b) and adipocytes (c). Numbers on the y-axes depict the number of proteins with the given motif in the dataset. Violin plot of the EV to cell abundance ratios (log₁₀) of all proteins in the dataset is included as a reference. Median value of all proteins in the dataset is represented as a dashed line. *P<0.05.

FIG. 12 In silico-identified EV-enriched domain annotations

(a) Venn diagram of statistically significant EV-enriched annotated domains in HEK293T, MSCs and adipocytes. Numbers in the diagram refer to the number of domains identified for each group. Violin plots representing the EV to cell abundance ratios (log₁₀) of proteins with statistically significant EV-enriched domains identified in HEK293T (b), MSCs (c) and adipocytes (d). Only EV-enriched annotated domains common across all three cell lines are shown. Numbers on the y-axes depict the number of proteins with the given domain in the dataset. Violin plot of the EV to cell abundance ratios (log₁₀) of all proteins in the dataset is included as a reference. Median value of all proteins in the dataset is represented as a dashed line. *P<0.05.

FIG. 13 Assessment of EV-loading features from bioinformatics analysis.

(a) Scheme of the GFP-fusion constructs that were assessed. (b) Percentage of GFP+ EVs quantified by single-vesicle analysis of conditioned media from HEK293T cells transfected with GFP-fusion constructs. (c) Median fluorescence intensity of GFP+ EVs. (d) Western blot of GFP-fusion constructs in HEK293T cells and EVs. Cell lysates and isolated EVs were blotted against ACTB (beta-actin) and ALIX respectively. Both cell lysates and isolated EVs were blotted against GFP. Data are presented as mean+SEM, n=3 replicates, *P<0.05, **P<0.01, ****P<0.0001.

FIG. 14 Western blot of Cre-fusion constructs.

(a) Western blot of Cre-fusion constructs from FIG. 4d,e in HEK293T EVs and cells. (b) Western blot of Cre-fusion constructs from FIG. 4f,g in HEK293T cells and EVs. Both cell lysates and isolated EVs were blotted against c-Myc. Cell lysates and isolated EVs were blotted against ACTB (beta-actin) and ALIX respectively. SC is a self-cleaving endopeptidase from Shewanella oneidensis. PTTG1IPDel has a deletion from amino acid 130 to 164. PTTG1IP-130-180aa has amino acids 130-180 duplicated on PTTG1IP C-terminus.

FIG. 15 PTTG1IP has a higher EV-loading capacity than CD63.

(a) Percentage of GFP+ EVs quantified by single-vesicle analysis. (b) Median fluorescence intensity of GFP+ EVs quantified by single-vesicle analysis of conditioned media from HEK293T cells transfected with PTTG1IP-GFP or CD63-GFP. (c) Western blot of GFP-fusion constructs in HEK293T cells and EVs. Cell lysates and isolated EVs were blotted against ACTB (beta-actin) and ALIX respectively. Both cell lysates and isolated EVs were blotted against GFP. Data are presented as mean+SEM, n=3 replicates, ***P<0.001.

FIG. 16 Amino acids 130-180 of PTTG1IP are able to induce cleavage in EVs when fused to CD63.

HEK293T cells were transfected with CD63-intein-Cre or CD63-3×PTTG1IP(130-180aa)-Cre, and Cre cleavage in the EVs was analysed. Western blot of CD63-intein-Cre and CD63-3×PTTG1IP(130-180aa)-Cre in HEK293T cells and EVs. Cell lysates and isolated EVs were blotted against Cre, ALIX and ACTB (beta-actin).

FIG. 17 Assessment of Cas9 reporter cell activation after Cas9 and sgRNA transfection.

GFP positive cells quantified by flow cytometry 48h after Cas9, sgRNA or Cas9 and sgRNA transfection to HEK293T stoplight reporter cells. Data are presented as mean+SEM, n=3 replicates, ****P<0.0001.

FIG. 18 Comparison of PTTG1IP and CD63 for EV-mediated delivery of Cas9.

GFP positive cells quantified by flow cytometry 48h after PTTG1IP-intein-Cas9 or CD63-intein-Cas9 EV-transfer to sgRNA transfected HEK293T stoplight reporter cells. Isolated EVs were transferred to recipient cells in 96-well plates (2×10⁹ EVs/ml). Data are presented as mean+SEM, n=2, **P<0.01.

FIG. 19 Uptake of targeted EVs displaying HER2 scFv in PTTG1IP N terminus by human breast cancer cell line SKBR3.

Luciferase activity (RLU, relative light units) in recipient cells after treatment with 2×10⁶ or 2×10⁵ RLU EVs derived from cells expressing PTTG1IP fused to HER2 scFv targeting domain in the N terminus and to Nluc in the C terminus or EVs derived from cells expressing PTTG1IP fused to only to Nluc. * p<0.05.

FIG. 20 Comparison of PTTG1IP and Lamp2B for loading of GFP.

(a) The bar chart shows the proportion of GFP positive EVs produced by HEK cells, as quantified by NanoFCM Flow, 72h after the HEK cells were transfected with a Lamp2B-GFP or a PTTG1IP-eGFP construct. The engineered PTTG1IP constructs are expressed in a comparable proportion of EVs to engineered Lamp2B constructs.

(b) The bar chart shows the median fluorescence (GFP) intensity in EVs produced by HEK cells, as quantified by flow cytometry, 72 h after transfection with a Lamp2B-GFP or a PTTG1IP-eGFP construct. The EVs produced by the cells transfected with the engineered PTTG1IP construct have a higher average GFP signal, indicating a higher level of expression, as compared to EVs produced by cells transfected with engineered Lamp2B construct.

FIG. 21 Comparison of Assessment of EV co-localisation of VSVG and engineered PTTG1IP or VSVG and engineered Lamp2B.

(a) The bar chart shows the proportion of GFP positive EVs produced by HEK cells, as quantified by NanoFCM Flow, 72h after the HEK cells were transfected with a VSVG construct and a Lamp2B-GFP, 06E07-Lamp2b-eGFP, IRVHH-Lamp2b-eGFP, PTTG1IP-eGFP, 06E07-PTTG1IP-eGFP or a IRVHH-PTTG1IP-eGFP construct. The engineered PTTG1IP constructs are expressed in a larger proportion of EVs produced by cells that were co-transfected with VSVG, as compared to engineered Lamp2B constructs.

(b) The bar chart shows the proportion of GFP and VSVG positive EVs produced by HEK cells, as quantified by NanoFCM Flow, 72h after the HEK cells were transfected with a VSVG construct and a Lamp2B-GFP, 06E07-Lamp2b-eGFP, IRVHH-Lamp2b-eGFP, PTTG1IP-eGFP, 06E07-PTTG1IP-eGFP or an IRVHH-PTTG1IP-eGFP construct. A larger proportion of the EVs that were produced by cells that were transfected with an engineered PTTG1IP constructs and a VSVG construct were double positive for GFP and VSVG, as compared with the EVs that were produced by cells that were transfected with an engineered Lamp2B construct and a VSVG construct.

FIG. 22 Comparison of PTTG1IP and Lamp2B for functional delivery of Cre Recombinase in vitro.

(a) The western blot image shows the level of Cre Recombinase expression and the level of VSVG expression in EVs produced by HEK cells, 72h after the cells were transfected with a VSVG construct and a Lamp2B-intein-Cre, 06E07-Lamp2b-intein-Cre, IRVHH-Lamp2b-intein-Cre, PTTG1IP-intein-Cre, 06E07-PTTG1IP-intein-Cre or a IRVHH-PTTG1IP-intein-Cre construct. Cre expression was increased in EVs produced by the cells that were transfected with the engineered PTTG1IP constructs, as compared with the EVs that were produced by cells that were transfected with an engineered Lamp2B construct and a VSVG construct. Alix is the loading control.

(b) The immunofluorescence images show B16 Traffic light cells. The nuclei of all the cells have been stained and the cells into which if Cre has been functionally delivered and has induced genetic recombination also express GFP in the cytoplasm. EVs were produced in HEK cells that were transfected with a VSVG construct and a Lamp2B-intein-Cre, a 06E07-Lamp2b-intein-Cre, an IRVHH-Lamp2b-intein-Cre, a PTTG1IP-intein-Cre, a 06E07-PTTG1IP-intein-Cre or an IRVHH-PTTG1IP-intein-Cre construct. The EVs were added to the B16 Traffic light cells and representative images were taken after 40 hours.

(c) The bar chart shows the percentage of the B16 Traffic light cells that are GFP positive 40 hours after the addition of EVs produced by HEK cells that had been transfected with a VSVG construct and a Lamp2B-intein-Cre, a 06E07-Lamp2b-intein-Cre, an IRVHH-Lamp2b-intein-Cre, a PTTG1IP-intein-Cre, a 06E07-PTTG1IP-intein-Cre or an IRVHH-PTTG1IP-intein-Cre construct. Addition of EVs produced by cells transfected with VSVG and engineered PTTG1IP constructs mediate improved functional delivery of the EV cargo, as compared to EVs produced by cells transfected with VSVG and engineered Lamp2b constructs.

### DETAILED DESCRIPTION OF THE INVENTION

The present disclosure is directed to an EV comprising a single pass EV transmembrane protein fused to a moiety on the surface of the EV and/or a cargo molecule. Surprisingly, such an EV may be efficiently loaded with cargo, and enable the effective delivery of the cargo, for example in vitro and/or in vivo. Advantageously, the efficiency of binding and delivery relative to existing proteins used for loading cargo to EVs, such as CD63 and/or Lamp2B, is drastically improved, in particular when a single pass EV transmembrane protein such as PTTG1IP or a variant or fragment thereof, is used. In addition, PTTG1IP allows for the effective production of EVs that co-express the engineered PTTG1IP construct alongside other exogenous constructs (for instance endosomal escape moieties such as VSVG or additional scaffold proteins carrying additional cargo molecules).

A further significant advantage of PTTG1IP is the surprising intrinsic cleavage activity that is present. Therefore, the present invention allows for significantly improved delivery of cargo using EVs, for example in a therapeutic setting.

### General Definitions

Unless defined otherwise, technical and scientific terms used herein have the same meaning as commonly understood by a person skilled in the art to which this invention belongs.

In general, the term “comprising” is intended to mean including but not limited to. For example, the phrase “a method comprising” particular steps should be interpreted to mean that the method includes those steps, but the method may comprise further steps. In some embodiments, “comprising” may be replaced by “consisting”.

The terms “around” or “about” or “˜” in relation to a reference numerical value and its grammatical equivalents as used herein can include the numerical value itself and a range of values plus or minus 10% from that numerical value. For example, the term “around” or “about” in relation to a reference numerical value can also include a range of values plus or minus 10%, 9%, 8%, 7%, 6%, 5%, 4%, 3%, 2%, or 1% from that value.

The singular forms “a”, “an”, and “the” include plural referents unless the content clearly dictates otherwise.

Embodiments and features described in connection with one of the aspects and/or embodiments of the present invention also apply mutatis mutandis to all the other aspects and/or embodiments of the invention. For example, the fusion proteins described herein (i.e. the single pass EV transmembrane protein fused to: (a) a moiety on the surface of the EV and/or (b) a cargo molecule), are to be understood to be disclosed, relevant, and compatible with all other aspects, teachings and embodiments herein, for instance aspects and/or embodiments relating to the methods for producing or purifying the EVs, or relating to the corresponding polynucleotide constructs described herein or the engineered EV-producing cells from which the EVs derive. Furthermore, certain embodiments described in connection with certain aspects, for instance the administration routes of the EVs comprising the therapeutic cargo molecule and optionally the fusion polypeptides, as described in relation to aspects pertaining to treating certain medical indications, may naturally also be relevant in connection with other aspects and/or embodiment such as those pertaining to the pharmaceutical compositions comprising such EVs. Furthermore, all polypeptides and proteins identified herein can be freely combined in fusion proteins using conventional strategies for fusing polypeptides. As a non-limiting example, the fusion protein described herein may be freely combined in any combination with one or more targeting moiety, endosomal escape moiety, additional cargo, albumin or albumin binding domain, additional protein, optionally combined with all other polypeptide domains, regions, sequences, peptides, groups herein, e.g. any multimerization domains, linker sequences, release domains, etc. Furthermore, when teachings herein refer to EVs in singular and/or to EVs as discrete natural nanoparticle-like vesicles it should be understood that all such teachings are equally relevant for and applicable to a plurality of EVs and populations of EVs. As a general remark, a therapeutic protein, binding protein for a therapeutic agent, targeting moiety, endosomal escape moiety, albumin or albumin binding domain or purification domain and all other aspects, embodiments, and alternatives in accordance with the present invention may be freely combined in any and all possible combinations without deviating from the scope and the gist of the invention. Furthermore, any polypeptide or polynucleotide or any polypeptide or polynucleotide sequences (amino acid sequences or nucleotide sequences, respectively) of the present invention may deviate considerably from the original polypeptides, polynucleotides and sequences as long as any given molecule retains the ability to carry out the desired technical effect associated therewith. As long as their biological properties are maintained the polypeptide and/or polynucleotide sequences according to the present application may deviate with as much as 50% (calculated using, for instance, BLAST or ClustalW) as compared to the native sequence, although a sequence identity or similarity that is as high as possible is preferable (for instance 60%, 70%, 80%, or e.g. 90% or higher). Standard methods in the art may be used to determine homology. For example, PILEUP and BLAST algorithms can be used to calculate homology or line up sequences. The combination (fusion) of e.g. several polypeptides implies that certain segments of the respective polypeptides may be replaced and/or modified and/or that the sequences may be interrupted by insertion of other amino acid stretches, meaning that the deviation from the native sequence may be considerable as long as the key properties (e.g. retaining its therapeutic effect, or ability to bind to a therapeutic cargo or to bind to albumin and therefore extend half-life, ability to traffic a fusion construct to an EV, targeting capabilities, etc.) are conserved. Similar reasoning thus naturally applies to the polynucleotide sequences encoding for such polypeptides. Any accession numbers or SEQ ID NOs mentioned herein in connection with peptides, polypeptides and proteins shall only be seen as examples and for information only, and all peptides, polypeptides and proteins shall be given their ordinary meaning as the skilled person would understand them. Thus, as above-mentioned, the skilled person will also understand that the present invention encompasses not merely the specific SEQ ID NOs and/or accession numbers referred to herein but also variants and derivatives thereof. All proteins, polypeptides, peptides, nucleotides and polynucleotides mentioned herein are to be construed according to their conventional meaning as understood by a skilled person.

The terms “genetically modified” and “genetically engineered” are used interchangeably herein and can mean, for example, that the EV, preferably an exosome, is derived from a genetically modified/engineered cell or is otherwise genetically engineered to express and/or modify the expression of proteins in the lumen, extravesicular membrane and/or displayed on the surface of the EV (e.g., exosome), which is typically incorporated into the EVs, preferably exosomes, produced by those cells. Such genetically engineered or genetically modified EVs do not occur in nature.

A “nucleic acid” refers to a polynucleotide and includes polyribonucleotides and poly-deoxyribonucleotides. Nucleic acids according to the present invention may include any polymer or oligomer of pyrimidine and purine bases, e.g., cytosine, thymine, and uracil, and adenine and guanine, respectively. Indeed, the present invention contemplates any deoxyribonucleotide or ribonucleotide component, and any chemical variants thereof. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, at least 8, at least 15 or at least 25 nucleotides in length, but may be up to 50, 100, 1000, 5000, 10000, 15000, or 20000 nucleotides long or a compound that specifically hybridises to a polynucleotide. Polynucleotides include sequences of DNA or RNA or mimetics thereof, which may be isolated from natural sources, recombinantly produced or artificially synthesised. A further example of a polynucleotide as employed in the present invention may be a peptide nucleic acid (PNA; see U.S. Pat. No. 6,156,501, which is hereby incorporated by reference in its entirety.) The invention also encompasses situations in which there is a non-traditional base pairing, such as Hoogsteen base pairing, which has been identified in certain tRNA molecules and postulated to exist in a triple helix. “Polynucleotide” and “oligonucleotide” are used interchangeably herein. It will be understood that when a nucleotide sequence is represented herein by a DNA sequence (e.g., A, T, G, and C), this also includes the corresponding RNA sequence (e.g., A, U, G, C) in which “U” replaces “T”.

As used herein, “polynucleotide” includes, for instance, cDNA, RNA, DNA/RNA hybrid, antisense RNA, siRNA, mRNA, guideRNA, ribozyme, genomic DNA, synthetic forms, and mixed polymers, both sense and antisense strands, and may be chemically or biochemically modified to contain non-natural or derivatised, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

The term “small molecule agent” or “small molecule” or “small molecule drug” or “small molecule therapeutic” are used interchangeably herein and shall be understood to relate to any molecular agent which may be used for the treatment and/or diagnosis of a disease and/or disorder, and also for modulating or changing e.g. the activity and/or the binding and/or the location of a binding protein. Small molecule agents are normally synthesized via chemical synthesis means, but may also be naturally derived, for instance via purification from natural sources, or may be obtained through any other suitable means or combination of techniques. A brief, non-limiting definition of a “small molecule” is any organic compound with a molecular weight of less than 900 g/mol (Dalton) that may in essentially any way regulate, impact, or influence a biological process. For the purposes of this invention, small molecules may be substantially larger than 900 g/mol, for instance 1500 g/mol, 3000 g/mol, or occasionally even larger. Overall, molecular weight and/or molecular size is not a defining factor behind what constitutes a small molecule agent. In fact, for the purposes of the present invention any agent that can be bound by a binding protein displayed on an EV is considered to be a “small molecule agent”.

All publications, patents and patent applications cited herein, whether supra or infra, are hereby incorporated by reference in their entirety.

### Extracellular Vesicles (EVs)

The terms “extracellular vesicle” or “EV” or “exosome” shall be understood to relate to any type of vesicle, or vesicle variant, that is, for instance, obtainable from a cell, for instance a microvesicle (e.g. any vesicle shed from the plasma membrane of a cell), an exosome (e.g. any vesicle derived from the endo-lysosomal pathway such as a multi-vesicular body), an apoptotic body (e.g. obtainable from apoptotic cells), ARRDC1 Mediated Microvesicle (ARIM), a microparticle (which may be derived from e.g. platelets), an ectosome (derivable from e.g. neutrophils and monocytes in serum), prostatosome (e.g. obtainable from prostate cancer cells), or a cardiosome (e.g. derivable from cardiac cells), etc. Furthermore, the said terms shall be understood to also relate to in some embodiments extracellular vesicle mimics, cellular membrane vesicles obtained through membrane extrusion or other techniques, etc. Essentially, the present invention may relate to any type of lipid-based structure (with vesicular morphology or with any other type of suitable morphology). It will be clear to the skilled artisan that when describing medical and scientific uses and applications of the EVs, the present invention normally relates to a plurality of EVs, i.e. a population of EVs which may comprise thousands, millions, billions or even trillions or even more EVs. In the same vein, the term “population” shall be understood to encompass a plurality of entities which together constitute such a population. In other words, individual EVs when present in a plurality constitute an EV population. Thus, naturally, the present invention pertains both to individual EVs and populations of EVs, as will be clear to the skilled person. Similar reasoning naturally applies to the genetically modified cells, i.e. both individual cells and populations of such cells.

Extracellular vesicles (EVs) are lipid bilayer-delimited particles that are naturally released from a cell and, unlike a cell, cannot replicate. EVs range in diameter from near the size of the smallest physically possible unilamellar liposome (around 20-30 nm) to as large as 10 m. Thus, the EVs can have a diameter of 30 nm-150 nm, 30 nm-250 nm, 30 nm-500 nm, 30 nm-1000 nm, 150 nm-250 nm, 150 nm-500 nm, 150 nm-1000 nm, 250 nm-500 nm, 250 nm-1000 nm or 500 nm-1000 nm. EVs can carry a variety of cargo, such as proteins, nucleic acids, lipids, metabolites, small molecule drugs, biological drugs such as antibodies, and/or organelles from the parent cell. Most cells that have been studied to date release EVs, including eukaryotic cells such as animal and plant cells, bacterial cells and fungal cells. In addition, EVs have also been isolated from physiological fluids, such as plasma, urine, amniotic fluid and malignant effusions. A wide variety of EV subtypes have been proposed, defined variously by size, biogenesis pathway, cargo, source, and function. EVs for use in accordance with the present invention can be derived from any suitable cell or physiological fluid.

During the last decade, a new paradigm of cell-to-cell communication has emerged involving EVs, with implications for both normal and pathological physiology. Given their unique biological and pharmacological characteristics, EVs have gained tremendous interest in understanding their roles in physiology, pathophysiology and drug delivery. Immunological inertness, ability to cross biological barriers, and to carry bioactive molecules are some of the attractive features of EVs that can be exploited in drug delivery applications. However, lack of efficient drug loading methods, and an incomplete understanding of EV biogenesis and uptake mechanisms remain critical challenges that need to be addressed. Current methods of loading therapeutic molecules into EVs such as electroporation, genetic engineering of host cells and chemical conjugation, are limited by low efficiency, toxicity and lack of scalability. Moreover, they produce a heterogeneous population of EVs that imposes complexity in understanding phenotypic effect of EVs in the targeted cells. To harness therapeutic potential of EVs, it is important to understand intracellular pathways of EV biogenesis, which will provide opportunities to exploit the natural characteristics of EVs for therapeutic applications. There is a growing interest in clinical applications of EVs as biomarkers and therapies alike.

In any one of the embodiments of the invention, the EV may be an exosome. Exosomes are produced in the endosomal compartment of most eukaryotic cells. The multivesicular body (MVB) is an endosome defined by intraluminal vesicles (ILVs) that bud inward into the endosomal lumen. If the MVB fuses with the cell surface (the plasma membrane), these ILVs are released as exosomes. In multicellular organisms, exosomes and other EVs are present in tissues and can also be found in biological fluids including blood, urine, and cerebrospinal fluid. They are also released in vitro by cultured cells into their growth medium. Since the size of exosomes is limited by that of the parent MVB, exosomes are generally thought to be smaller than most other EVs, from about 20 to several hundred nm in diameter: around the same size as many lipoproteins but much smaller than cells.

EVs may form aggregates. Such aggregates arise through covalent and/or non-covalent interactions between molecules on the surface of the EV that result in two or more discrete EVs associated with each other such that the associated EVs substantially move together as one unit when in bulk solution. In a preferred embodiment, aggregation of EVs is minimized or eliminated, such that EVs exist as substantially discrete entities.

It will be clear to the skilled artisan that when describing medical and scientific uses and applications of the EVs, the present invention normally relates to a plurality of EVs, i.e. a population of EVs which may comprise thousands, millions, billions or even trillions of EVs. As can be seen from the experimental section below, EVs may be present in concentrations such as 10⁵, 108, 10¹⁰, 10¹¹, 10¹², 10¹³, 10¹⁴, 10¹⁵, 10¹⁸, 10²⁵, 10³⁰ EVs (often termed “particles”) per unit of volume (for instance per ml or per litre), or any other number larger, smaller or anywhere in between. In the same vein, the term “population”, which may e.g. relate to an EV comprising a certain protein shall be understood to encompass a plurality of entities which together constitute such a population. In other words, individual EVs when present in a plurality constitute an EV population. Thus, naturally, the present invention pertains both to individual EVs and populations comprising EVs, as will be clear to the skilled person. The dosages of EVs when applied in vivo may naturally vary considerably depending on the disease to be treated, the administration route, the activity and effects of additional constituents (e.g. an albumin binding domain (ABD)), therapeutic cargo, any targeting moieties present on the EVs, the pharmaceutical formulation, etc.

### Cell Sources

Generally, EVs may be derived from essentially any cell source, be it a primary cell source or an immortalized cell line. The EV source cells may be any embryonic, foetal, and adult somatic stem cell types, including induced pluripotent stem cells (iPSCs) and other stem cells derived by any method, as well as any adult cell source. The source cells per the present invention may be select from a wide range of cells and cell lines, for instance mesenchymal stem or stromal cells (MSCs) (obtainable from e.g. bone marrow, adipose tissue, Wharton's jelly, perinatal tissue, chorion, placenta, tooth buds, umbilical cord blood, skin tissue, etc.), fibroblasts, amnion cells and more specifically amnion epithelial cells optionally expressing various early markers, myeloid suppressor cells, M2 polarized macrophages, adipocytes, endothelial cells, fibroblasts, etc. Cell lines of particular interest include human umbilical cord endothelial cells (HUVECs), human embryonic kidney (HEK) cells such as a HEK293T cell, endothelial cell lines such as microvascular or lymphatic endothelial cells, erythrocytes, erythroid progenitors, chondrocytes, MSCs of different origin, amnion cells, amnion epithelial (AE) cells, any cells obtained through amniocentesis or from the placenta, airway or alveolar epithelial cells, fibroblasts, endothelial cells, etc. Also, immune cells such as B cells, T cells, NK cells, macrophages, monocytes, dendritic cells (DCs) are also within the scope of the present invention, and essentially any type of cell which is capable of producing EVs is also encompassed herein. When treating neurological diseases, one may contemplate to utilise as source cells e.g. primary neurons, astrocytes, oligodendrocytes, microglia, and neural progenitor cells. When treating neuromuscular diseases, one may contemplate to utilise as source cells e.g. myoblasts or myotubes. The source cell may be either allogeneic, autologous, or even xenogeneic in nature to the patient to be treated, i.e. the cells may be from the patient himself or from an unrelated, matched or unmatched donor. In certain contexts, allogeneic cells may be preferable from a medical standpoint, as they could provide immuno-modulatory effects that may not be obtainable from autologous cells of a patient suffering from a certain indication. For instance, in the context of treating systemic, peripheral and/or neurological inflammation, allogeneic MSCs or AEs may be preferable as EVs obtainable from such cells may enable immuno-modulation via e.g. macrophage and/or neutrophil phenotypic switching (from pro-inflammatory M1 or N1 phenotypes to anti-inflammatory M2 or N2 phenotypes, respectively). In one preferred embodiment, EVs are derived from HEK293T cells, MSCs or adipocytes. The cell lines from which EVs are derived may be adherent or suspension cells and may be generated as stable cell lines or single clones.

In particular, the present invention relates to cells which have been stably modified to comprise at least one monocistronic, bicistronic or multicistronic polynucleotide construct according to the invention (as defined above) encoding a fusion protein of the invention. Such cells may be stably or transiently transfected with the polynucleotides according to the present invention to render them engineered EV producing cells. The cells of the present invention may be a monoclonal cell or a polyclonal cell line.

Preferred producer cells according to the present invention may be a: HEK cell, a HEK293 cell, a HEK293T cell, an MSC, in particular a WJ-MSC cell or a BM-MSC cell, a fibroblast, an amnion cell, an amnion epithelial cell, CEVEC's CAP® cells, a placenta-derived cell, a cord blood cell, an immune system cell, an endothelial cell, an epithelial cell or any other cell type, wherein said cells may be for instance adherent cells, suspension cells, and/or suspension-adapted cells. In a most preferred embodiment, the producer cells are HEK293 cells.

### Single Pass EV Transmembrane Protein

A single pass EV transmembrane protein is any protein and/or peptide that spans the lipid membrane of an EV, such as the lipid bilayer of an EV, only once. As compared to other types of EV transmembrane proteins (e.g. tetraspanins), single pass EV proteins are associated with a number of advantages. For instance, moieties and/or proteins of interest may be presented on the surface of the EV by fusion onto the (N-)terminus of a single-pass EV protein, whereas with tetraspanin EV protein, more complex and restricted loop engineering is required. Hence engineering for surface-display is easier and there is increased likelihood of successful display and high levels of loading being achieved. Engineering at the CTD (c-terminal domain) and/or NTD (n-terminal domain) of single pass transmembrane proteins provides much more flexibility as compared to engineering into the loops of a multipass transmembrane protein, specifically larger cargos (including those cargos requiring protein linkers to allow for optimal activity) can be engineered into the construct without affecting the protein folding and membrane insertion which often restricts the utility of engineering into the loops of a multipass transmembrane protein. This is particularly the case where the moiety or protein of interest (POI) being engineered is of a large size, for instance larger than 30 kDa, larger than 40 kDa, larger than 50 kDa or larger than 75 kDa.

The single pass EV transmembrane protein may be expressed in the host cell from which the EV is isolated from. Alternatively, or in addition, the single pass EV transmembrane protein may be expressed in a separate cell and added to an isolated EV. The single pass EV transmembrane protein may preferably be expressed in the host cell from which the EV is isolated from. Expression may be natural and/or recombinant expression. The single pass EV transmembrane protein may be a protein that is naturally associated with the EV and/or host cell from which the EV is isolated. Alternatively, the single pass EV transmembrane protein may be a protein that is not naturally associated with the EV and/or host cell from which the EV is isolated.

The single pass EV transmembrane protein typically comprises three domains: the extracellular domain, the transmembrane domain, and the intracellular domain. The transmembrane domain is generally the smallest at typically around 25 amino acid residues and generally forms an alpha helix inserted into the membrane. The extracellular domain is typically larger than the intracellular domain and is often globular, whereas many intracellular domains have relatively high disorder. An intracellular domain protein may naturally function as a monomer. Alternatively or additionally, dimerization or higher-order oligomerization may confer biological function. The single pass EV transmembrane protein may have at least one globular domain situated at one or both sides of EV membrane, for example comprised in the extracellular domain and/or the intracellular domain).

The single pass EV transmembrane protein may be selected from one or more of a Type I, Type II, Type III and Type IV single pass EV transmembrane protein. Type I single pass EV transmembrane proteins have their N-terminus on the extracellular side of the membrane and a signal peptide that has been cleaved off. Type II single pass EV transmembrane proteins have their N-terminus on the cytoplasmic side of the membrane and a transmembrane helix located close to the N-terminus, where it functions as an anchor. Type III single pass EV transmembrane proteins have their N-terminus on the extracellular side of the membrane and no signal peptide. Type IV single pass EV transmembrane proteins have their N-terminus on the cytoplasmic side of the membrane, and a transmembrane helix located close to the C-terminus where it functions as an anchor.

The single pass EV transmembrane protein may have a maximum molecular weight of 10 kDa, 15 kDa, 20 kDa, 25 kDa, 30 kDa, 40 kDa, 50 kDa, 75 kDa, 100 kDa, 250 kDa, 500 kDa, 750 kDa or 1000 kDa. The maximum molecular weight of the EV transmembrane protein may preferably be 100 kDa, more preferably 50 kDa, and most preferably 25 kDa. The molecular weight of the single pass EV transmembrane protein may be 10 kDa to 15 kDa, 10 kDa to 20 kDa, 10 kDa to 25 kDa, 10 kDa to 30 kDa, 10 kDa to 40 kDa, 10 kDa to 50 kDa, 10 kDa to 75 kDa, 10 kDa to 100 kDa, 10 kDa to 250 kDa, 10 kDa to 500 kDa, 10 kDa to 750 kDa, 10 kDa to 1000 kDa, 15 kDa to 20 kDa, 15 kDa to 25 kDa, 15 kDa to 30 kDa, 15 kDa to 40 kDa, 15 kDa to 50 kDa, 15 kDa to 75 kDa, 15 kDa to 100 kDa, 15 kDa to 250 kDa, 15 kDa to 500 kDa, 15 kDa to 750 kDa, 15 kDa to 1000 kDa, 20 kDa to 25 kDa, 20 kDa to 30 kDa, 20 kDa to 40 kDa, 20 kDa to 50 kDa, 20 kDa to 75 kDa, 20 kDa to 100 kDa, 20 kDa to 250 kDa, 20 kDa to 500 kDa, 20 kDa to 750 kDa, 20 kDa to 1000 kDa, 25 kDa to 30 kDa, 25 kDa to 40 kDa, 25 kDa to 50 kDa, 25 kDa to 75 kDa, 25 kDa to 100 kDa, 25 kDa to 250 kDa, 25 kDa to 500 kDa, 25 kDa to 750 kDa, 25 kDa to 1000 kDa, 30 kDa to 40 kDa, 30 kDa to 50 kDa, 30 kDa to 75 kDa, 30 kDa to 100 kDa, 30 kDa to 250 kDa, 30 kDa to 500 kDa, 30 kDa to 750 kDa, 30 kDa to 1000 kDa, 40 kDa to 50 kDa, 40 kDa to 75 kDa, 40 kDa to 100 kDa, 40 kDa to 250 kDa, 40 kDa to 500 kDa, 40 kDa to 750 kDa, 40 kDa to 1000 kDa, 50 kDa to 75 kDa, 50 kDa to 100 kDa, 50 kDa to 250 kDa, 50 kDa to 500 kDa, 50 kDa to 750 kDa, 50 kDa to 1000 kDa, 75 kDa to 100 kDa, 75 kDa to 250 kDa, 75 kDa to 500 kDa, 75 kDa to 750 kDa, 75 kDa to 1000 kDa, 100 kDa to 250 kDa, 100 kDa to 500 kDa, 100 kDa to 750 kDa, 100 kDa to 1000 kDa, 250 kDa to 500 kDa, 250 kDa to 750 kDa, 250 kDa to 1000 kDa, 500 kDa to 750 kDa, 500 kDa to 1000 kDa, or 750 kDa to 1000 kDa.

The single pass EV transmembrane protein may comprise one or more post translational modification. The post translational modification may be selected from one or more of isoglutamyl cysteine, O-linked GalNAc threonine, a GPI-anchor, N-linked GlcNAc complex asparagine, heparan sulfate, cleavage on pair of basic residues, a proteoglycan, pyrrolidone carboxylic acid, O-linked xylose, a zymogen, phosphoserine such as by FAM20C, N-linked GlcNAc asparagine, at least one interchain disulphide bond, a disulfide bond, a glycoprotein, hydroxyproline, glycosylation, ADP-ribosylarginine such as by cholera toxin, palmitate, O-linked glycosylation, phosphothreonine such as by PKC, a lipoprotein, removed in mature form, cysteine methyl ester, lipidation, S-modification, prenylation, S-geranylgeranyl cysteine, ubiquitination, myristoylation, sumolyation and phosphorylation. More preferably, the post translational modification may be selected from one or more of glycosylation such as N-linked GlcNAc asparagine, prenylation, ubiquitination, myristoylation, sumoylation, phosphorylation, lipidation, hydroxyproline, pyrolidone carboxylic acid and kinase specific phosphorylation. Most preferably, the post translational modification may be N-linked GlcNAc asparagine.

The single pass EV transmembrane protein may be selected from a protein from a suitable database, such as from the ExoCarta database (exocarta.org), or a variant or fragment thereof. In one embodiment, the single pass EV transmembrane protein is not vesicular stomatitis virus G (VSVG) protein or a variant or fragment thereof. The single pass EV transmembrane protein may comprise:
(a) a polypeptide sequence having at least 80%, at least 90%, at least 95% or at least 100% sequence identity to SEQ ID NO: 1
(b) a polypeptide sequence having at least 80%, at least 90%, at least 95% or at least 100% sequence identity to any one of SEQ ID NOs: 2 to 5;
(c) at least 10, at least 20 or at least 30 contiguous amino acid residues from the polypeptide sequence of any one of SEQ ID NOs: 1 to 5; and/or
(d) pituitary tumour-transforming gene 1 interacting protein (PTTG1IP), or a variant or fragment thereof, optionally wherein the variant or fragment thereof retains the intrinsic cleavage activity of PTTG1IP, for example the intrinsic cleavage activity of SEQ ID NO: 5.

The number of single pass EV transmembrane protein molecules associated with 25 each EV may be 1-10, 1-100, 1-500, 1-1000, 1-3000, 1-5000, 1-10,000, 100-500, 100-1000, 100-3000, 100-5000, 100-10,000, 500-1000, 500-3000, 500-5000, 500-10,000, 1000-3000, 1000-5000, 1000-10,000, 3000-5000, 3000-10,000 or 5000-10,000. The number of single pass EV transmembrane protein molecules associated with each EV may be at least about 10 molecules, at least about 100 molecules, at least about 500 molecules, at least about 1000 molecules, at least about 3000 molecules, at least about 5000 molecules or at least about 10,000 molecules. The number of single pass EV transmembrane proteins associated with each EV according to the invention is preferably increased compared to the number of single pass EV transmembrane proteins associated with each EV in a wild type setting. In this context, a wild type setting may refer to an unmodified naturally occurring EV and/or an unmodified naturally occurring single pass EV transmembrane protein. The number of single pass EV transmembrane proteins associated with each EV according to the present invention may be increased, when compared to a wild type setting, by at least 1.25 fold, at least 1.5 fold, at least 2 fold, at least 5 fold, at least 10 fold, at least 25 fold, at least 50 fold, at least 100 fold, at least 250 fold, at least 500 fold, at least 1000 fold, at least 2500 fold, at least 5000 fold, or at least 10,000 fold.

An EV or EV composition of the invention may comprise or consist of a homogenous or substantially homogenous population of single pass EV transmembrane proteins (i.e., the single pass EV transmembrane proteins associated with the EV may be the same or substantially the same). Alternatively, an EV or EV composition of the invention may comprise or consist of a heterogenous population of single pass EV transmembrane proteins (i.e., two or more different single pass EV transmembrane proteins may be associated with the same EV). Such a heterogeneous population may comprise at least 2, at least 3, at least 4, at least 5, at least 6, at least 7, at least 8, at least 9 or at least 10 different single pass EV transmembrane proteins associated with the same EV. For example, 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10 different single pass EV transmembrane proteins may be associated with the same EV.

### Moiety on the Surface of the EV

A moiety may be associated with the surface of the EV, and therefore exposed to the environment that is external to the EV. The term “associated” may be used in its broadest sense to describe a moiety associated with an EV such that the moiety and EV move substantially together as one unit when in bulk solution. The moiety may, for example, be associated with the outer leaflet of the EV membrane, such as the outer lipid bilayer of the EV. The moiety may be associated through a non-covalent interaction. Examples of non-covalent interactions include electrostatic interactions such as ionic interactions, hydrogen bonding and halogen bonding. Examples of non-covalent interactions also include Van der Waals forces and hydrophobic effects. However, in a preferable embodiment, the moiety is fused to the surface of the EV by a covalent interaction, preferably a covalent interaction with the single pass EV transmembrane protein of the invention. For example, the moiety on the surface of the EV may be fused to the transmembrane portion of the single pass EV transmembrane protein through the design of a suitable construct, such that the moiety on the surface of the EV is covalently linked through the peptide backbone to the transmembrane protein of the single pass EV transmembrane protein.

One or more linkers can optionally be included between two protein sequences that are to be fused, such as between the moiety and the single pass EV transmembrane protein. The linker physically separates and preferably preserves the functionality of one or both of the proteins that are fused. For example, the presence of a linker, may allow flexibility (for instance, to avoid steric hinderance from components of the EV corona) and enables the fusion protein to be positioned optimally for display on the surface of the EV or luminally according to what is required. Linkers according to the invention are useful in providing increased flexibility, improving pharmacokinetics (PK), increasing expression and improving biological activity of the fusion polypeptide constructs, and also to the corresponding polynucleotide constructs, and may also be used to ensure avoidance of steric hindrance and maintained functionality of the fusion polypeptides. Exemplary linkers according to the present invention include glycine or serine linkers which increase stability or flexibility such as (GGGGS)n (n=1, 2, 4) or (Gly)6, (Gly)8, rigid linkers such as (EAAAK)n (n=1-3, and A(EAAAK)4ALEA(EAAAK)4A, bending linkers (XP)ₙ or cleavable linkers such as disulphide, protease sensitive sequences. The linker may preferably be 10 amino acids or fewer in length. In some aspects, the linker is 9, 8, 6, 5, 4, 3, 2 amino acids in length or shorter. The linker can include any amino acids, such as Ala, Pro, Cys, and Gly. The linker may be present between PTTG1IP and, for example GFP, intein, Cre or a self-cleaving sequence. Alternatively or in addition, the linker may be present between CD63 and a self-cleaving sequence, and/or between a self-cleaving sequence and Cre. In one specific embodiment of the invention, the linker may be DPPVAT (SEQ ID NO: 6) or a variant or fragment thereof.

Alternatively, or in addition to, the linker, the fusion protein may comprise a multimerization domain. Multimerization domains may be homomultimerization domains or heteromultimerization domains. The multimerization domains may be a dimerization domain, a trimerization domain, a tetramerization domain, or any higher order of multimerization domain. Multimerization domains enable dimerization, trimerization, or any higher order of multimerization of the fusion polypeptides, which increases the sorting and trafficking of the fusion polypeptides into EVs and may also contribute to increase the yield of vesicles produced by EV-producing cells. Exemplary multimerization domains include: leucine zipper, fold-on domain, fragment X, collagen domain, 2G12 IgG homodimer, mitochondrial antiviral-signaling protein CARD filament, Cardiac phospholamban transmembrane pentamer, parathyroid hormone dimerization domain, Glycophorin A transmembrane, HIV Gp41 trimerisation domain, HPV45 oncoprotein E7 C-terminal dimer domain, and any combination thereof. In one embodiment, a multimierization domain is not present.

The moiety may be present in the host cell from which the EV is isolated from. For example, a protein moiety may be expressed in the host cell from which the EV is isolated from. Alternatively, or in addition, the moiety may be added to an isolated EV. For example, the protein moiety may be expressed in a different cell from the cell that the EV is isolated from, and/or the moiety may be produced artificially such as in a suitable cell-free system. Expression may be natural and/or recombinant expression. The moiety may be one that is naturally associated with the EV and/or host cell from which the EV is isolated. Alternatively, moiety may be one that is not naturally associated with the EV and/or host cell from which the EV is isolated.

The moiety on the surface of the EV may be selected from any suitable molecule. For example, the moiety may be selected from one or more of a peptide and/or protein, a targeting peptide and/or targeting protein which binds to a molecule present on a cell to be targeted, a therapeutic moiety such as a receptor decoy, an endosomal escape moiety, an enzyme, a nuclease, a CRISPR-associated protein (Cas) such as SaCas9, SpCas9, Cas9, Cas12, Cas13 and variants and fusions thereof, a Transcription activator-like effector nucleases (TALEN) and variants and fusions thereof, a meganuclease and variants and fusions thereof, a zinc finger nuclease and variants and fusions thereof, an antibody and/or antigen-binding variant or fragment thereof, a single chain variable fragment (scFv), VHH, a nanobody, a nucleic acid-binding protein and/or peptide, a RNA- and/or DNA-binding protein, viral-binding protein, a small molecule drug, a nucleic acid, a nucleic acid analogue, an unnatural nucleic acid, gRNA, miRNA, shRNA, siRNA, piRNA, PMO, DNA and a DNA plasmid encoding a therapeutic peptide and/or protein. In one preferable embodiment, the moiety is a targeting moiety. A targeting moiety is any moiety which confers tropism or increases the tropism of the EV to a desired target, such as a desired cell type or tissue. In another preferable embodiment, the moiety has therapeutic utility.

The moiety on the surface of the EV may alternatively or in addition be selected from enzymes, receptors such as decoy receptors, membrane proteins, transporters, cytokines, antigens, neoantigens, immune effector molecules, ribonuclear proteins, nucleic acid binding proteins, antibodies, nanobodies, antibody fragments, antibody-drug conjugates, gene editing proteins such as CRISPR effector proteins including Cas proteins, transcription activator-like effector nucleases (TALENs), meganucleases, viral capsids or viral capsid binding proteins (in particular an AAV capsid, such as. AAV8 or AAV9). Immune effector molecules are particularly useful in the case of EVs loaded with a viral (e.g. AAV or lentiviral) cargo but may equally be used where EV is loaded with any cargo according to the invention. The immune effector may act to reduce immunogenicity of the EV. In some embodiments, the immune effector functions stimulate immune inhibitors. In other embodiments, the immune effector functions inhibit immune stimulating molecules. In some embodiments, ABD-EV comprises molecules that stimulate immune inhibitors and molecules that inhibit immune stimulating molecules. Exemplary immune effector molecules include, but are not limited to, one or more of CTLA4, B7-1, B7-2, PD-1, PD-L1, PD-L2, CD28, or VISTA. In some embodiments, the envelope comprises CTLA4 and PD-L1, CTLA and PD-L2 CTLA-4 and VISTA, PD-L1 and PD-L2, PD-L1 and VISTA, PD-L2 and VISTA, CTLA4 and PD-L1 and PD-L2, CTLA4 and PD-L1 and VISTA, CTLA4 and PD-L2 and VISTA, PD-L1 and PD-L2 and VISTA, or CTLA4 and PD-L1 and PD-L1 and VISTA. The immune effector molecule may form part of the fusion construct, or may form part of a separate fusion protein construct comprising an immune effector molecule fused to any EV protein according to the present invention.

The moiety on the surface of the EV, such as genetic material on the surface of the EV, may be modified. For example, the genetic material may comprise: (i) single stranded 2′-O-methyl ribose modifications, single stranded 2′-O-methoxy-ethyl (MOE) ribose modifications, single stranded phosphoroamidate backbone chemistry, single stranded methylphosphonate backbone chemistry and/or single stranded phospohorothioate backbone chemistry; (ii) antisense modified oligonucleotides, antisense modified oligonucleotides comprising 2′-O-Me ribose modifications, antisense modified oligonucleotides comprising peptide nucleic acids, antisense modified oligonucleotides designed to induce exon skipping, antisense modified oligonucleotides which inhibit hairpin loops, and/or trans-splicing antisense modified oligonucleotides; and/or (iii) unnatural oligonucleotides selected from 2′-O-Me ribose modified oligonucleotides, morpholino, thio-nucleotides, chemically modified siRNAs and/or peptide nucleic acids.

In a preferred embodiment, the moiety on the surface of the EV is a targeting peptide and/or targeting protein. In one embodiment the targeting peptide and/or targeting protein is a moiety which binds to a target expressed on the surface of a cell or tissue to be targeted. In a most preferred embodiment, the targeting moiety targets the liver, the heart, the brain or neuronal tissue, hepatocytes, cardiomyocytes, cardiac smooth muscle cells, sensory neurons, motor neurons, interneurons or glia cells.

In a further preferred embodiment, the moiety on the surface of the EV is moiety that is capable of binding a therapeutic cargo. In one embodiment, the therapeutic cargo is an RNA, in particular a guide RNA; a Cas, in particular Cas9 or Cas12; or a virus, in particular an adeno-associated virus. In one embodiment, the therapeutic cargo has been modified to include a domain that renders it capable of binding to the moiety on the surface of the EV. In one embodiment, the moiety on the surface of the EV may be an RNA-binding protein or protein domain, for instance a PUF domain. In an alternative embodiment, the moiety on the surface of the EV may be a protein binding protein or protein domain such as an Fc-binder, a nanobody or a VHH. In a particularly preferred embodiment, the moiety on the surface of the EV is a nanobody or VHH that is capable of binding a Cas protein, in particular Cas9 or Cas12, or an adeno-associated virus, in particular an AAV capsid, preferably an AAV8 or an AAV9 capsid.

### Targeting

The EV of the present invention may be targeted, for example to a desired cell type or tissue. This targeting is achieved by expressing on the surface of the EV a targeting moiety which binds to the target, such as a target expressed on the surface of a cell or tissue to be targeted. The targeting moiety may be any moiety identified above. The targeting moiety may be a peptide which may be expressed as a fusion with a transmembrane protein typically expressed on the surface of the EV, such as fusion to the single pass EV transmembrane protein of the invention.

In more detail, the EV of the invention can be targeted, for example to particular cell types or tissues, by expressing on their surface a targeting moiety such as a peptide. Suitable peptides are those which bind to cell surface moieties such as receptors or their ligands found on the cell surface of the cell to be targeted. Examples of suitable targeting moieties are short peptides, scFv and complete proteins, so long as the targeting moiety can be expressed on the surface of the EV and preferably does not interfere with cargo carrying capacity of the EV. Peptide targeting moieties may typically be less than 100 amino acids in length, for example less than 50 amino acids in length, less than 30 amino acids in length, to a minimum length of 10, 5 or 3 amino acids. For example, peptide targeting moieties may be 3 to 5 amino acids, 3 to 10 amino acids, 3 to 30 amino acids, 3 to 50 amino acids, 3 to 100 amino acids, 5 to 10 amino acids, 5 to 30 amino acids, 5 to 50 amino acids, 5 to 100 amino acids, 10 to 30 amino acids, 10 to 50 amino acids, 10 to 100 amino acids, 30 to 50 amino acids, 30 to 100 amino acids or 50 to 100 amino acids in length.

Targeting moieties can be selected to target particular cells, subcellular locations, tissues, organs or other bodily compartments. Organs and cell types that may be targeted include: the brain, neuronal cells, the blood brain barrier, muscle tissue, the eye, lungs, liver, kidneys, heart, stomach, intestines, pancreas, red blood cells, white blood cells including B cells and T cells, lymph nodes, bone marrow, spleen and cancer cells. Alternatively, or in addition, targeting moieties may target a diseased tissue such as a tumour, for example HER2-positive breast tumours. In a preferred embodiment, the EV are targeted to brain tissue. In a most preferred embodiment, the targeting moiety targets the liver, the heart, the brain or neuronal tissue, hepatocytes, cardiomyocytes, cardiac smooth muscle cells, sensory neurons, motor neurons, interneurons or glia cells.

Specific examples of targeting moieties include muscle specific peptide, discovered by phage display, to target skeletal muscle, a 29 amino acid fragment of Rabies virus glycoprotein (RVG) that binds to the acetylcholine receptor or a fragment of neural growth factor that targets its receptor to target neurons, the secretin peptide that binds to the secretin receptor can be used to target biliary and pancreatic epithelia. As an alternative, immunoglobulins and their derivatives, including scFv antibody fragments and VHHs can also be expressed as a fusion protein to target specific antigens. As an alternative, natural ligands for receptors can be expressed as fusion proteins to confer specificity, such as NGF which binds NGFR and confers neuron-specific targeting. The peptide targeting moiety may be expressed on the surface of the EV by expressing it as a fusion protein with an EV transmembrane protein. A number of proteins are known to be associated with EVs; that is they are incorporated into the EV as it is formed. The preferred proteins for use in targeting the EVs of the present invention are those which are transmembrane proteins or fused to transmembrane proteins, such as the single pass EV transmembrane protein of the invention. Other examples of targeting moieties include brain targeting moieties such as rabies virus glycoprotein (RVG), nerve growth factor (NGF) which binds to NGF-receptor, melanotransferrin and the FC5 Peptide and muscle targeting moieties such as Muscle Specific Peptide (MSP).

The targeting moiety may be expressed as part of a fusion protein with the single pass transmembrane protein according to the present invention alternatively the targeting moiety may be engineered into the EV by fusion to a classical EV protein. The EV proteins which is comprised in the fusion proteins as per the present invention may be selected from any classical EV protein, such as one selected from the group comprising the following non-limiting examples: CD9, CD53, CD63, CD81, CD54, CD50, FLOT1, FLOT2, CD49d, CD71, CD133, CD138, CD235a, ALIX, Syntenin-1, Syntenin-2, LAMP-2B, syndecan-1, syndecan-2, syndecan-3, syndecan-4, a TSPAN such as TSPAN1, TSPAN2, TSPAN3, TSPAN4, TSPAN5, TSPAN6, TSPAN7, TSPAN8, TSPAN9, TSPAN10, TSPAN11, TSPAN12, TSPAN13, TSPAN14, TSPAN15, TSPAN16, TSPAN17, TSPAN18, TSPAN19, UPK1B, UPK1A, PRPH2, ROM1, TSPAN31, TSPAN32, TSPAN33, CD37, CD82, CD151, CD231, CD102, NOTCHI, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD 274, CD362, COL6A1, AGRN, EGFR, GAPDH, GLUR2, GLUR3, HLA-DM, HSPG2, L1CAM, LAMB1, LAMC1, ARRDC1, PDGFRN, ATP2B2, ATP2B3, ATP2B4, BSG, IGSF2, IGSF3, IGSF8, ITGB1, ATP1A2, ATP1A3, ATP1A4, ITGA4, SLC3A2, ATP1A1, ATP1B3, ATP2B1, LFA-1, LGALS3BP, Mac-1 alpha, Mac-1 beta, MFGE8, SLIT2, STX3, TCRA, TCRB, TCRD, TCRG, VTI1A, VTI1B, an integrin such as ITGAD, ITGAE, ITGAL, ITGAM, ITGAV, ITGAX, ITGA1, ITGA2, ITGA2B, ITGA3, ITGA4, ITGA5, ITGA6, ITGA7, ITGA8, ITGA9, ITGA10, ITGA11, ITGBL1, ITGB1, ITGB2, ITGB3, ITGB4, ITGB5, ITGB6, ITGB7, ITGB8, PTGFRN, BASP1 and any other EV proteins, and any combinations, derivatives, domains, variants, mutants, or regions thereof.

The EV of the present invention may be targeted to a desired cell type or tissue. For example, the EV of the present invention may be targeted to a cancer cell and/or the blood-brain-barrier (BBB). When present in the blood, EV of the present invention may cross the BBB. At least 0.01%, at least 0.1%, at least 1%, at least 2%, at least 5%, at least 10%, at least 20%, at least 25%, at least 30%, at least 40%, at least 50%, at least 60%, at least 70%, at least 80%, at least 90%, at least 95% or at least 99% of the total number of EVs delivered into a patient may be targeted to the desired cell type or tissue and/or cross the BBB.

### Cargo Molecule

The EV may be loaded with a cargo molecule (i.e. cargo). The terms “load”, “loaded”, “loading”, “onto an EV” and “into an EV” may be used in their broadest sense to describe a cargo molecule associated with an EV such that the EV and its cargo molecule move substantially together as one unit when in bulk solution. Thus, the cargo molecule may be encapsulated in the interior (i.e. within the lumen) of the EV. Alternatively, or in addition, the cargo molecule may be present on the surface (i.e. outside) of the EV. The cargo molecule may be associated with the inner and/or outer membrane of the EV, such as the lipid bilayer of the EV. Alternatively, or in addition, the cargo molecule may be associated with an EV transmembrane protein, such as the single pass EV transmembrane protein of the invention.

In a preferable embodiment, association of the cargo molecule with the EV is through a covalent bond with the EV transmembrane protein, such as with the single pass EV transmembrane protein of the invention. In such embodiments, a linker can optionally be included between the cargo molecule and the single pass EV transmembrane protein. The linker is preferably 10 amino acids or fewer in length. In some aspects, the linker is 9, 8, 6, 5, 4, 3, 2 amino acids in length or shorter. The linker can include any amino acids, such as Ala, Pro, Cys, and Gly. The linker may be as defined above. Alternatively or in addition, association of the cargo molecule with the EV may be through a non-covalent interaction, such as a non-covalent interaction with the EV membrane. Examples of non-covalent interactions include electrostatic interactions such as ionic interactions, hydrogen bonding and halogen bonding. Examples of non-covalent interactions also include Van der Waals forces, hydrophobic effects and ionic interactions. The cargo molecule may be inside and/or outside the EV depending upon the orientation of its association, for example the orientation of its fusion with the single pass EV transmembrane protein of the invention. When the cargo molecule is inside the EV, it may also be possible to reduce or eliminate recognition by the innate immune system of the cargo molecule and thus reduce or eliminate acute inflammatory responses associated with the naked delivery of the cargo molecule.

The cargo molecule may be present in the host cell from which the EV is isolated from. For example, a cargo molecule may be expressed in the host cell from which the EV is isolated from. Alternatively, or in addition, the cargo molecule may be added to an isolated EV. For example, the cargo molecule may be a protein cargo molecule which may be expressed in a different cell from the cell that the EV is isolated from, and/or the cargo molecule may be produced artificially such as in a suitable cell-free system. Expression may be natural and/or recombinant expression. The cargo molecule may be one that is naturally associated with the EV and/or host cell from which the EV is isolated (i.e. an endogenous cargo molecule). Alternatively, the cargo molecule may be one that is not naturally associated with the EV and/or host cell from which the EV is isolated (i.e. an exogenous cargo molecule).

The cargo molecule may be selected from any suitable molecule. For example, the cargo molecule may be selected from one or more of a therapeutic cargo, a peptide and/or protein, an enzyme, a nuclease, a CRISPR-associated protein (Cas) such as SaCas9, SpCas9, Cas9, Cas12, Cas13 and variants and fusions thereof, a Transcription activator-like effector nucleases (TALEN) and variants and fusions thereof, a meganuclease and variants and fusions thereof, a zinc finger nuclease and variants and fusions thereof, an antibody and/or antigen-binding variant or fragment thereof, a single chain variable fragment (scFv), VHH, a nanobody, a nucleic acid-binding protein and/or peptide, a RNA- and/or DNA-binding protein, a small molecule drug, a nucleic acid, a nucleic acid analogue, an unnatural nucleic acid, gRNA, miRNA, shRNA, siRNA, piRNA, PMO, DNA and a DNA plasmid encoding a therapeutic peptide and/or protein. In a preferred embodiment, the cargo molecule has therapeutic utility.

The cargo molecule may alternatively or in addition be selected from enzymes, receptors such as decoy receptors, membrane proteins, transporters, cytokines, antigens, neoantigens, immune effector molecules, ribonuclear proteins, nucleic acid binding proteins, antibodies, nanobodies, antibody fragments, antibody-drug conjugates, gene editing proteins such as CRISPR effector proteins including Cas proteins, transcription activator-like effector nucleases (TALENs), meganucleases.

The cargo molecule may alternatively or in addition be selected from antibodies, intrabodies, nanobodies, single chain variable fragments (scFv), VHHs, affibodies, bi- and multispecific antibodies or binders including bispecific T-cell engagers (BiTEs), receptors, ligands, transporters, enzymes for e.g. ERT or gene editing, tumour suppressors, viral or bacterial inhibitors, cell component proteins, DNA repair inhibitors, nucleases, proteinases, integrases, transcription factors, growth factors, apoptosis inhibitors and inducers, toxins (for instance pseudomonas exotoxins), structural proteins, neurotrophic factors such as NT3/4, brain-derived neurotrophic factor (BDNF) and nerve growth factor (NGF) and its individual subunits such as the 2.5S beta subunit, ion channels, membrane transporters, proteostasis factors, proteins involved in cellular signaling, translation- and transcription related proteins, nucleotide binding proteins, protein binding proteins, lipid binding proteins, glycosaminoglycans (GAGs) and GAG-binding proteins, metabolic proteins, cellular stress regulating proteins, inflammation and immune system regulating proteins such as cytokines and inhibitors of such cytokines (cytokines may include: CXCL8, GMCSF, interleukins including: IL-1 family, IL-2, IL-4, IL-6, IL-6-like, IL-9, IL-10, IL12, IL-13, IL-17, Interferons including INF-alpha/beta/gamma, TNF family members, CD40 and CD40L, TRAIL, and TGF-beta family) mitochondrial proteins, and heat shock proteins, etc. The cargo protein may be a reporter protein such as green fluorescent protein (GFP) or nanoLuc. In one preferred embodiment, the encoded protein is a CRISPR-associated (Cas) polypeptide (such as Cas9) with intact nuclease activity which is associated with (i.e. carries with it) an RNA strand that enables the Cas polypeptide to carry out its nuclease activity in a target cell once delivered by the peptide. The CRISPR (Clustered Regularly Interspaced Short Palindromic Repeats) components according to the present invention include CRISPR components that are derived from any bacterial source. The CRISPR components may come from class 1 or class 2, specifically the Cas type may be Cas type I, II, III, IV, V or VI. The specific Cas protein may be Cas9, Cas12 (Cas12a or Cas12b), C2c2, Cpf1, Cas10, Cas13 (cas13a, Cas13b or Cas13c), Cas3, a Cas14 protein, a CasX protein, or a CasY protein, CasMINI, or SuperFi-Cas9. The CRISPR protein may be a CRISPR nuclease, a CRISPR nickase, or a nuclease deficient CRISPR variant. Alternatively, in another preferred embodiment, the Cas polypeptide may be catalytically inactive, to enable targeted genetic engineering. Yet another alternative may be any other type of CRISPR effector such as the single RNA guided endonuclease Cpf1. The inclusion of Cpf1 is a particularly preferred embodiment of the present invention, as it cleaves target DNA via a staggered double-stranded break. Cpf1 may be obtained from species such as Acidaminococcus or Lachnospiraceae. In yet another exemplary embodiment, the Cas polypeptide may also be fused to a transcriptional activator (such as the P3330 core protein), to specifically induce gene expression.

The cargo molecule may alternatively or in addition be selected from the group comprising enzymes or transporters for lysosomal storage disorders, for instance glucocerebrosidases such as imiglucerase, alpha-galactosidase, alpha-L-iduronidase, iduronate-2-sulfatase and idursulfase, arylsulfatase, galsulfase, acid-alpha glucosidase (GAA), sphingomyelinase, galactocerebrosidase, galactosylceramidase, ceramidase, alpha-N-acetylgalactosaminidase, beta-galactosidase, lysosomal acid lipase, acid sphingomyelinase, NPC1, NPC2, heparan sulfamidase, N-acetylglucosaminidase, heparan-a-glucosaminide-N-acetyltransferase, N-acetylglucosamine 6-sulfatase, galactose-6-sulfate sulfatase, galactose-6-sulfate sulfatase, hyaluronidase, alphaN-acetyl neuraminidase, GlcNAc phosphotransferase, mucolipin1, palmitoylprotein thioesterase, tripeptidyl peptidase I, palmitoyl-protein thioesterase 1, tripeptidyl peptidase 1, battenin, linclin, alpha-D-mannosidase, beta-mannosidase, aspartylglucosaminidase, alpha-L-fucosidase, cystinosin, cathepsin K, sialin, LAMP2, and hexoaminidase.

The cargo molecule may alternatively or in addition be selected from the group comprising enzymes associated with Urea cycle disorders including N-Acetylglutamate synthase, carbamoyl phosphate synthetase, ornithine transcarbamoylase, argininosuccinic acid synthase, argininosuccinic acid lyase, arginase, mitochondrial ornithine transporter, citrin, y+L amino acid transporter 1, uridine monophosphate synthase UMPS.

In other preferred embodiments, the cargo molecule may be e.g. an intracellular protein that modifies inflammatory responses, for instance epigenetic proteins such as methylases and bromodomains, or an intracellular protein that modifies muscle function, e.g. transcription factors such as MyoD or Myf5, proteins regulating muscle contractility e.g. myosin, actin, calcium/binding proteins such as troponin, or structural proteins such as Dystrophin, mini-dystrophin, micro-dystrophin, utrophin, titin, nebulin, dystrophin-associated proteins such as dystrobrevin, syntrophin, syncoilin, desmin, sarcoglycan, dystroglycan, sarcospan, agrin, and/or fukutin. The cargo is typically a protein or peptide of human origin unless indicated otherwise by their name, any other nomenclature, or as known to a person skilled in the art, and they can be found in various publicly available databases such as Uniprot, RCSB, etc.

The cargo molecule may alternatively or in addition be an antigen/neoantigen, optionally wherein the antigen/neoantigen is suitable for use in cancer immunotherapy. Any antigen/neoantigen may be incorporated into the EVs of the present invention. The antigens may be suitable for raising immune responses against pathogens such as bacteria, viruses, funguses or the antigen may be a tumour antigen useful in eliciting an immune response against a tumour for cancer immunotherapy. There may be one or more antigens/neoantigens present in any EV according to the invention. The one or more antigens/neoantigens may be endogenous/autologous (coming from the subject itself) or exogenous/allogenic (coming from another subject) or in the case of more antigens/neoantigens being incorporated into/onto the EVs the antigens/neoantigens may be any mix of autologous/allogenic antigens. Preferably the antigens are autologous. Moreover, the one or more antigens/neoantigens may have any origin such as e.g. viral or bacterial or may be a tumour antigen and furthermore may be immunostimulatory or immunosuppressive or a combination thereof. The antigen/neoantigen maybe be useful in the treatment of any disease by immunotherapy. The treatment of cancer by immunotherapy is a particularly preferred embodiment. Where the antigen is a neo-antigen it may be identified by sequencing of a tumour to identify the neo-antigen. Exemplary tumour antigens may be: Alphafetoprotein (AFP), Carcinoembryonic antigen (CEA), CA-125, MUC-1, Epithelial tumour antigen (ETA), Melanoma-associated antigen (MAGE), WT-1, NY-ESO-1, LY6K, IMP3, DEPDC1, CDCA-1,abnormal products of ras, p53, KRAS, or NRAS, CTAG1B, Peptides derived from chromosomal translocations such as BCR-ABL or ETV6-AML1, viral antigens such as peptides from HPV-related cancers, peptides derived from proteins such as tyrosinase, gp100/pmel17, Melan-A/MART-1, gp75/TRP1, or TRP2, and overexpressed antigens such as MOK (RAGE-1), ERBB2 (HER2/NEU). Where the therapeutic cargo is an antigen or neoantigen the EV or pharmaceutical composition comprising the EV may optionally further comprise an adjuvant. Where the antigen is administered with an adjuvant to stimulate the immune response the adjuvant may be: an inorganic compound: such as aluminium hydroxide, aluminium phosphate, calcium phosphate hydroxide, a mineral oil such as paraffin oil, bacterial products such as killed bacteria Bordetella pertussis, Mycobacterium bovis, toxoids, a nonbacterial organic such as squalene, a detergent such as Quil A, a plant saponin, a cytokine such as IL-1, IL-2, IL-12, or RIBI (muramyl dipeptides) or ISCOM (immunostimulating complexes) such as STING (stimulator of interferon genes) agonists which can include cyclic dinucleotides. Such adjuvants may protect the therapeutic EV from rapid dispersal by sequestering it in a local deposit, or they may contain substances that stimulate the host to secrete factors that are chemotactic for macrophages and other components of the immune system. Adjuvants that can be incorporated to a vaccine are well-known by the person skilled in the art and will be selected, in such a way that they do not negatively affect the immunological activity of the EV. Where the adjuvant is a protein (for example a cytokine) in specific embodiments the adjuvant may be incorporated into the EV itself as part of the fusion protein.

Where the cargo molecule is a genetic cargo molecule such as a nucleic acid, the cargo molecule may have utility in gene therapy and/or gene editing. Nucleic acids are routinely used in gene therapy for the replacement of non-functional genes and for neutralization of disease-causing mutations via RNA interference (RNAi) effector molecules such as miRNAs, shRNAs and siRNAs. Exemplary genetic material cargos include: messenger RNA (mRNA), circular mRNA, Doggybone® DNA (dbDNA®), linear DNA, circular DNA, plasmid DNA, linear RNA, circular RNA, self-amplifying RNA or DNA, a viral genome either “naked” or within a capsid or a modified version of any of the above. As naked DNA and RNA are difficult to deliver in vivo due to rapid clearance, nucleases, lack of organ-specific distribution and low efficacy of cellular uptake, specialized gene delivery vehicles, such as viral vectors and cationic liposomes, are usually used for delivery. Loading EVs with genetic material cargo has a number of advantages, such as overcoming mutagenic integration associated with viruses such as lentiviruses; and inflammatory toxicity and rapid clearance associated with liposomes.

The genetic material to be loaded into the EVs is chosen on the basis of the desired effect of that genetic material on the cell into which it is intended to be delivered and the mechanism by which that effect is to be carried out. For example, the genetic material may be useful in gene therapy, for example in order to express a desired gene in a cell or group of cells. Such genetic material is typically in the form of plasmid DNA or viral vector encoding the desired gene and operatively linked to appropriate regulatory sequences such as promoters, enhancers and the like such that the plasmid DNA is expressed once it has been delivered to the cells to be treated. Exemplary viral cargos include: a viral vector which is an AAV vector or a lentiviral vector. In some embodiments, the viral vector is an AAV vector. In some embodiments, the AAV vector comprises a capsid from human AAV serotype AAV1, AAV2, AAV3, AAV4, AAV5, AAV6, AAV7, AAV8, AAV9, AAV 10, AAV11 or AAV12. In some embodiments, the AAV vector comprises an AAV viral genome comprising inverted terminal repeat (ITR) sequences from human AAV serotype AAV1, AAV2, AAV3, AAV4, AAV5, AAV6, AAV7, AAV8, AAV9, r AAV10. In some embodiments, the AAV capsid and the AAV ITR are from the same serotype or from different serotypes. In some embodiments the viral vector comprises a viral capsid and a viral genome, the viral genome comprising one or more heterologous transgenes. In preferred embodiments, the heterologous transgene encodes a polypeptide or protein. The protein encoded with in the viral genome may be any one of the protein cargos according to the invention allowing the viral cargo to act as a gene replacement therapy.

Examples of diseases susceptible to gene therapy include genetic diseases such as haemophilia B (Factor IX), cystic fibrosis (CTFR), Phenylketonuria (PKU), ALS and spinal muscular atrophy (SMN-1).

Genetic material can also be used in gene silencing. Such gene silencing may be useful in therapy to switch off aberrant gene expression or in animal model studies to create single or more genetic knock outs. Typically, such genetic material is provided in the form of siRNAs. For example, RNAi molecules including siRNAs can be used to knock down DMPK with multiple CUG repeats in muscle cells for treatment of myotonic dystrophy. In other examples, plasmids expressing shRNA that reduces the mutant Huntington gene (htt) responsible for Huntington's disease can be delivered with neuron specific exosomes. Other target genes include BACE-1 for the treatment of Alzheimer's disease. Some cancer genes may also be targeted with siRNA or shRNAs, such as ras, c-myc and VEGFR-2. Brain targeted siRNA loaded exosomes may be particularly useful in the silencing of BACE-1 in Alzheimer's disease, silencing of alpha-synuclein in Parkinson's disease, silencing of htt in Huntingdon's disease and silencing of neuronal caspase-3 used in the treatment of stroke to reduce ischaemic damage.

Genetic material can also be used for example in immunisation to express one or more antigens against which it is desired to produce an immune response. Thus, the nucleic acid cargo to be loaded into the EV can encode one or more antigens against which is desired to produce an immune response, including but not limited to tumour antigens, antigens from pathogens such as viral, bacterial or fungal pathogens. It will of course be apparent that the antigens may alternatively, or additionally, be a protein cargo molecule, such as a protein cargo molecule fused to the single pass EV transmembrane protein of the invention.

Genetic material, for example nucleic acid cargo molecules and/or nucleic acid that may be bound to a RNA/DNA binding protein or exogenously loaded into the exosome may in addition be selected from the group comprising shRNA, siRNA, saRNA, miRNA, an anti-miRNA, mRNA, modified mRNA, gRNA, pri-miRNA, pre-miRNA, circular RNA, piRNA, tRNA, rRNA, snRNA, lncRNA, ribozymes, mini-circle DNA, plasmid DNA, RNA/DNA vectors, trans-splicing oligonucleotides, splice-switching oligonucleotides, CRISPR guide strands, morpholinos (PMO) antisense oligonucleotides (ASO), peptide-nucleic acids (PNA), a viral genome and viral genetic material (for instance a naked AAV genome), but essentially any type of nucleic acid molecule can be delivered by the EVs of the present invention. Both single-stranded and double-stranded nucleic acid molecules are within the scope of the present invention, and the nucleic acid molecule may be naturally occurring (such as RNA or DNA) or may be a chemically synthesised RNA and/or DNA molecule which may comprise chemically modified nucleotides such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, thionucleotides, phosphoramidate, PNA, PMO, etc.

The cargo molecule, such as the genetic material, may be modified. For example, the genetic cargo molecule may comprise: (i) single stranded 2′-O-methyl ribose modifications, single stranded 2′ methoxy-ethyl backbone chemistry, single stranded phosphoroamidate backbone chemistry, single stranded methylphosphonate backbone chemistry and/or single stranded phospohorothioate backbone chemistry; (ii) antisense modified oligonucleotides, antisense modified oligonucleotides comprising 2′-O-Me ribose modifications, antisense modified oligonucleotides comprising peptide nucleic acids, antisense modified oligonucleotides designed to induce exon skipping, antisense modified oligonucleotides which inhibit hairpin loops, and/or trans-splicing antisense modified oligonucleotides; and/or (iii) unnatural oligonucleotides selected from 2′-O-Me ribose modified oligonucleotides, morpholino, thio-nucleotides, chemically modified siRNAs and/or peptide nucleic acids.

When the genetic material is an mRNA, the mRNA may be a naturally or non-naturally occurring mRNA. An mRNA may include one or more modified nucleobases, nucleosides, or nucleotides. A nucleobase of an mRNA is an organic base such as a purine or pyrimidine or a derivative thereof. A nucleobase may be a canonical base (e.g., adenine, guanine, uracil, and cytosine) or a non-canonical or modified base including one or more substitutions or modifications including but not limited to alkyl, aryl, halo, oxo, hydroxyl, alkyloxy, and/or thio substitutions; one or more fused or open rings; oxidation; and/or reduction. Thus, a nucleobase may be selected from the non-limiting group consisting of adenine, guanine, uracil, cytosine, 7-methylguanine, 5-methylcytosine, 5-hydroxymethylcytosine, thymine, pseudouracil, dihydrouracil, hypoxanthine, and xanthine. A nucleoside of an mRNA is a compound including a sugar molecule (e.g., a 5-carbon or 6-carbon sugar, such as pentose, ribose, arabinose, xylose, glucose, galactose, or a deoxy derivative thereof) in combination with a nucleobase. A nucleoside may be a canonical nucleoside (e.g., adenosine, guanosine, cytidine, uridine, 5-methyluridine, deoxyadenosine, deoxyguanosine, deoxycytidine, deoxyuridine, and thymidine) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase and/or sugar component. A nucleotide of an mRNA is a compound containing a nucleoside and a phosphate group or alternative group (e.g., boranophosphate, thiophosphate, selenophosphate, phosphonate, alkyl group, amidate, and glycerol). A nucleotide may be a canonical nucleotide (e.g., adenosine, guanosine, cytidine, uridine, 5-methyluridine, deoxyadenosine, deoxyguanosine, deoxycytidine, deoxyuridine, and thymidine monophosphates) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase, sugar, and/or phosphate or alternative component. A nucleotide may include one or more phosphate or alternative groups. For example, a nucleotide may include a nucleoside and a triphosphate group. A “nucleoside triphosphate” (e.g., guanosine triphosphate, adenosine triphosphate, cytidine triphosphate, and uridine triphosphate) may refer to the canonical nucleoside triphosphate or an analog or derivative thereof and may include one or more substitutions or modifications as described herein. For example, “guanosine triphosphate” should be understood to include the canonical guanosine triphosphate, 7-methylguanosine triphosphate, or any other definition encompassed herein. An mRNA may include a 5′ untranslated region, a 3′ untranslated region, and/or a coding or translating sequence, which is translated to create the fusion protein of the present invention. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine. In some embodiments, an mRNA may include a 5′ cap structure, a chain terminating nucleotide, a stem loop, a polyA sequence, and/or a polyadenylation signal. A cap structure or cap species is a compound including two nucleoside moieties joined by a linker which caps the mRNA at its 5′ end, and which may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, iri27′03′GpppG, iri27′03′GppppG, iri27′02′GppppG, m7Gpppm7G, m73′dGpppG, iri27′03′GpppG, iri27′03′GppppG, and m27 02′GppppG. An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. An mRNA may instead or additionally include a stem loop, such as a histone stem loop. A stem loop may include 1, 2, 3, 4, 5, 6, 7, 8, 9 or more nucleotide base pairs. For example, a stem loop may include 4, 5, 6, 7, 8, 9 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. The modified mRNA of the present invention may comprise in addition to the coding region (which codes for the fusion protein and which may be codon-optimized) one or more of a stem loop, a chain terminating nucleoside, miRNA binding sites, a polyA sequence, a polyadenylation signal, 3′ and/or 5′ untranslated regions (3′ UTRs and/or 5′ UTRs) and/or a 5′ cap structure. As abovementioned, various nucleotide modifications are preferably incorporated into the mRNA to modify it for increased translation, reduced immunogenicity, and increased stability. Suitable modified nucleotides include but are not limited to N1-methyladenosine (m1A), N6-methyladenosine (m6A), 5-methylcytidine (m5C), 5-methyluridine (m5U), 2-thiouridine (s2U), 5-methoxyuridine (5moU), pseudouridine (ψ), N1-methylpseudouridine (m1ψ). Among these mRNA modifications, m5C and ψ are the most preferred as they reduce the immunogenicity of mRNA as well as increase the translation efficiency in vivo. In preferred embodiments of the present invention, the composition herein comprises a modified mRNA as the polynucleotide cargo, wherein the mRNA is modified with at least 50% m5C and 50% ψ or m1ψ, preferably at least 75% m5C and 75% ψ or m1ψ, and even more preferably 90% m5C and 90% ψ or m1ψ, or even more preferably 100% modification using m5C and ψ or m1ψ.

The cargo molecule may be a small molecule. Non-limiting examples of small molecules include anticancer agents such as doxorubicin, methotrexate, 5-fluorouracil or other nucleoside analogues such as cytosine arabinoside, proteasome inhibitors such as bortezomib, or kinase inhibitors such as imatinib or seliciclib, or NSAIDs such as naproxen, aspirin, or celecoxib, antibiotics such as heracillin, or antihypertensives such as ACE inhibitors such as enalapril, ARBs such as candesartan, cyclic dinucleotides, etc. The present invention is naturally applicable also to other small molecules without departing from the gist of the invention, as would be clear to a person skilled in the art.

In a most preferred embodiment, the cargo is an RNA, in particular a guide RNA; a Cas, in particular Cas9 or Cas12; or a virus, in particular an adeno-associated virus. In one embodiment, the cargo has been modified to include a domain that renders it capable of binding to an RNA binding protein or a protein binding protein, for surface or luminal loading onto/into an EV.

### Binding Proteins

In certain embodiments, the moiety on the surface of the EV and/or cargo molecule may be a binding protein, such a binding protein for a therapeutic agent. Such binding proteins may be for instance RNA or DNA binding proteins, viral-binding proteins, small molecule binding proteins or Fc-binding proteins. In some embodiments the binding protein may be comprised in the fusion polypeptide. Alternatively, the binding protein may be present as a second protein, which may be expressed on a separate construct.

In an embodiment, the binding protein is a nucleic acid binding protein (NA-binding protein) such as an RNA or DNA binding protein. In these embodiments nucleic acid cargos which are loaded into the EVs by the binding of the nucleic acid to the NA-binding protein. Non-limiting examples of NA-binding proteins are Ago2, Dicer, Drosha, DGCR8, hnRNPA1, hnRNPA2B1, DDX4, ADAD1, DAZL, ELAVL4, IGF2BP3, SAMD4A, TDP43, FUS, FMR1, FXR1, FXR2, EIF4A13, the MS2 coat protein, as well as any domains, parts or derivates, thereof. More broadly, particular subclasses of RNA-binding proteins and domains, e.g. mRNA binding proteins (mRBPs), pre-rRNA-binding proteins, tRNA-binding proteins, small nuclear or nucleolar RNA-binding proteins, non-coding RNA-binding proteins, miRNA-binding proteins, shRNA-binding proteins and transcription factors (TFs). Furthermore, various domains and derivatives may also be used as the NA-binding domain to transport an NA cargo into EVs. Non-limiting examples of RNA-binding domains include small RNA-binding domains (RBDs) (which can be both single-stranded and double-stranded RBDs (ssRBDs and dsRBDs) such as DEAD, KH, GTP_EFTU, dsrm, G-patch, IBN_N, SAP, TUDOR, RnaseA, MMR-HSR1, KOW, RnaseT, MIF4G, zf-RanBP, NTF2, PAZ, RBM1CTR, PAM2, Xpo1, Piwi, CSD, and Ribosomal_L7Ae. Such RNA-binding domains may be present in a plurality, alone or in combination with others, and may also form part of a larger RNA-binding protein construct as such, as long as their key function (i.e. the ability to transport an NA cargo of interest, e.g. an mRNA or a short RNA) is maintained.

In preferred embodiments the present invention relates to two groups of NA-binding domains, namely PUF proteins and CRISPR-associated polypeptides (Cas), specifically Cas9, Cas6 and Cas13, as well as various types of NA-binding aptamers. The present invention uses the term PUF proteins to encompass all related proteins and domains of such proteins (which may also be termed PUM proteins), for instance human Pumilio homolog 1 (PUM1), PUMx2 or PUFx2 which are duplicates of PUM1, etc., or any NA-binding domains obtainable from any PUF (PUM) proteins. PUF proteins are typically characterized by the presence of eight consecutive PUF repeats, each of approximately 40 amino acids, often flanked by two related sequences, Csp1 and Csp2. Each repeat has a ‘core consensus’ containing aromatic and basic residues. The entire cluster of PUF repeats is required for RNA binding. The PUF proteins as per the present invention can be natural or engineered to bind anywhere in an RNA molecule, or alternatively one can choose PUF proteins with different binding affinities for different sequences and engineer the RNA molecule to contain said sequence. There is furthermore engineered and/or duplicated PUF domains that bind 16-nucleotides in a sequence-specific manner, which can also be utilized to increase the specificity for the NA cargo molecule further. Hence the PUF domain can be modified to bind any sequence, with different affinity and sequence length, which make the system highly modular and adaptable for any RNA cargo molecule as per the present invention. PUF proteins and regions and derivatives thereof that may be used as NA-binding domains as per the present invention include the following non-limiting list of PUF proteins: FBF, FBF/PUF-8/PUF-6, -7, -10, all from C. elegans; Pumilio from D. melanogaster; Puf5p/Mpt5p/Uth4p, Puf4p/Yg1014wp/Yg1023p, Puf5p/Mpt5p/Uth4p, Puf5p/Mpt5p/Uth4p, Puf3p, all from S. cerevisiae; PufA from Dictyostelium; human PUM1 (Pumilio 1, sometimes known also as PUF-8R) and any domains thereof, polypeptides comprising NA-binding domains from at least two PUM1, any truncated or modified or engineered PUF proteins, such as for instance PUF-6R, PUF-9R, PUF-1OR, PUF-12R, and PUF-16R or derivatives thereof, and X-Puf1 from Xenopus. Particularly suitable NA-binding PUFs as per the present invention includes the following: PUF 531, PUF mRNA loc (sometimes termed PUFengineered or PUFeng), and/or PUFx2, (sequences of which are available in PCT/EP2018/080681) and any derivatives, domains, and/or regions thereof. The PUF/PUM proteins are highly advantageous as they may be selected to be of human origin. Furthermore, as is the case with the PUF proteins, Cas proteins such as Cas6, Cas9 and Cas13 are highly preferred examples of releasable NA-binding domains which bind with suitable affinity to NA cargo molecules, thereby enabling a releasable, reversible attachment of the Cas protein to the NA cargo. As with the PUF-based NA-binding domains, the Cas proteins represent a releasable, irreversible NA-binding domain with programmable, modifiable sequence specificity for the target NA cargo molecule, enabling higher specificity at a lower total affinity, thereby allowing for both loading of the NA cargo into EVs and release of the NA cargo in a target location.

In some embodiments the moiety on the surface of the EV and/or cargo molecule may be a viral-binding protein, such as: proteins capable of binding to adeno-associated virus (AAV), for instance the AAV-receptor, or proteins capable of binding to other types of viruses such as lenti-viruses. Generally any protein capable of binding to a viral coat/envelope protein or capable of binding to a viral genome is within the scope of the invention. Exemplary viral-binding proteins include: AAVR GPR108, syndecans and albumin. Other exemplary viral cargos include: a viral vector which is an AAV vector or a lentiviral vector. In some embodiments, the viral vector is an AAV vector. In some embodiments, the AAV vector comprises a capsid from human AAV serotype AAV1, AAV2, AAV3, AAV4, AAV5, AAV6, AAV7, AAV8, AAV9, AAV 10, AAV11 or AAV12. In some embodiments, the AAV vector comprises an AAV viral genome comprising inverted terminal repeat (ITR) sequences from human AAV serotype AAV1, AAV2, AAV3, AAV4, AAV5, AAV6, AAV7, AAV8, AAV9, r AAV10. In some embodiments, the AAV capsid and the AAV ITR are from the same serotype or from different serotypes. In some embodiments, the viral vector is a lentiviral vector. In some embodiments, the lentiviral vector is derived from human immunodeficiency virus, a simian immunodeficiency virus or a feline immunodeficiency virus. In some embodiments, the lentiviral vector is non-replicating. In some embodiments, the lentiviral vector is non-integrating. In some embodiments the viral vector comprises a viral capsid and a viral genome, the viral genome comprising one or more heterologous transgenes. In preferred embodiments, the heterologous transgene encodes a polypeptide or protein. The protein encoded with in the viral genome may be any one of the protein cargos according to the invention allowing the viral cargo to act as a gene replacement therapy. In a particularly preferred embodiment, the viral-binding protein is a nanobody or VHH that is capable of binding an AAV capsid, preferably an AAV8 and/or an AAV9 capsid.

In some embodiments the moiety on the surface of the EV and/or cargo molecule may be a small molecule binding protein, such as any protein, polypeptide, or peptide (i.e. any molecule comprising a sequence of amino acids) to which a small molecule agent can be attached, via non-covalent or covalent attachment, or via a combination of both covalent and non-covalent interactions. The combination of a binding protein and a small molecule agent is herein described using terms such as “binding protein-small molecule conjugate” or “binding protein-small molecule drug conjugate” or “binding protein-small molecule agent conjugate”, or just “conjugate”. The binding protein may play several different roles: it may for instance be (i) a carrier and/or delivery modality which is primarily meant to transport a small molecule agent attached to it, (ii) a targeting agent to direct trafficking of the EV carrying the binder protein-small molecule conjugate to a particular location, (iii) a therapeutically active protein which becomes therapeutically active or inactive through the attachment of a small molecule, which may have agonistic or antagonistic effects, (iv) a signalling protein which together with or without its small molecule cargo may exert or contribute to a cellular and/or bodily change and a related therapeutic and/or prophylactic effect, (v) a protein carrying out or catalyzing a particular reaction only when brought into the proximity of another protein, etc. The binding protein may further contribute to a bodily and/or cellular action or activity and a related therapeutic effect by releasing the small molecule agent in a suitable location, or it may contribute to such effects by retaining the small molecule agent bound to it. An example of the first case is when the binding protein releases the small molecule drug inside a target cell after EV-mediated delivery, whereas an example of the second case is the delivery of an antibody-small molecule drug conjugate into a tumour.

In some embodiments the moiety on the surface of the EV and/or cargo molecule may be a protein capable of binding to an Fc domain, also known as an Fc-binding protein. The terms “Fc binding polypeptide” and “Fc binding protein” and “Fc binder” and “Fc-binding protein” and “binder” are used interchangeably herein and shall be understood to relate to any protein, polypeptide, or peptide (i.e. any molecule comprising a sequence of amino acids) which can bind an Fc domain. Typically, the Fc binding polypeptides of the present invention are derived from various sources that are either human or non-human (e.g. mammal sources, bacteria, etc.), they have high affinity for Fc domains of various antibody isotypes, subtypes, and species (for instance IgG (as non-limiting examples in the case of IgG, lgG1, lgG2, lgG3, lgG4, lgG2a, lgG2d, and/or lgG2c), IgA, IgM, IgM, IgD, etc.), and they can be fused to EV proteins. Non-limiting examples of Fc binding polypeptides in accordance with the present invention include, in addition to other Fc binding polypeptides mentioned through the present application, Protein A, Protein G, Protein A/G, Z domain, ZZ domain, human FCGRI, human FCGRIIA, human FCGRIIB (as a non-limiting example the accession number 31994), human FCGRIIC (as a non-limiting example the accession number 31995), human FCGRIIIA (as a non-limiting example the accession number P08637), human FCGR3B (as a non-limiting example the accession number 075015), human FCAMR, human FCERA, human FCAR, mouse FCGRI, mouse FCGRIIB, mouse FCGRIII, mouse, mouse FCGRn, and various combinations, derivatives, or alternatives thereof.

The terms “Fc containing protein” and “protein comprising an Fc domain” and “Fc domain-containing protein” and “Fc domain containing protein” and “Fc domain protein” and similar terms are used interchangeably herein and shall be understood to relate to any protein, polypeptide, or peptide (i.e. any molecule comprising a sequence of amino acids) which comprises an Fc domain, either naturally or as a result of engineering of the protein in question to introduce an Fc domain. Fc stands for “fragment crystallizable” or “fragment constant”, which is the name of the tail regions of antibodies. Fc domains can however also be created and used on other proteins, not only antibodies. Non-limiting examples of such Fc domain-containing proteins include antibodies and antibody derivatives, Fc-modified decoy receptors (such as CD24-Fc or CD52-Fc) and/or signal transducers such as interleukin decoy receptors for IL1, IL2, IL3, IL4, IL5, IL6 (such as the signal transducer gp130 (as a non-limiting example the accession number P40189)), IL7, IL8, TL9, IL10, IL1 1, IL12, IL13, IL14, IL15, IL17 (such as IL17R, with as a non-limiting example the accession number Q96F46), TL23 (such as IL23R, with as a non-limiting example the accession number Q5VWK5), etc., Fc domain-containing bi- and multi-specific binders, any type of Fc domain-containing receptors or ligands, Fc domain-modified enzymes for e.g. enzyme replacement therapy or gene editing, nucleases such as Cas and Cas9 onto which an Fc domain has been grafted, tumour suppressors fused to Fc domains, etc. suitable Fc domains that may be fused with a protein natively lacking an Fc domain include the following non-limiting examples: human IGHM (as a non-limiting example the accession number P01871), human IGHA1 (as a non-limiting example the accession number P01876), human IGHA2 (as a non-limiting example the accession number P01877), human IGKC (as a non-limiting example the accession number P01834), human IGHG1 (as a non-limiting example the accession number P01857), human IGHG2 (as a non-limiting example the accession number P01859), human IGHG3 (as a non-limiting example the accession number P01860), human IGHG4 (as a non-limiting example the accession number P01861), human IGHD (as a non-limiting example the accession number P01880), human IGHE (as a non-limiting example the accession number P01854). All accession numbers referred to herein are UniProtKB accession numbers.

The present invention is also directed to nanoparticle complexes comprising any binding protein according to the invention bound to the corresponding binding partner (such as albumin, an Fc containing protein, a nucleic acid, a virus or a small molecule). In this embodiment some or all of the EVs are bound to their corresponding binding partner.

The present invention also relates to pharmaceutical compositions comprising the nanoparticle complexes of the invention combined with a pharmaceutically acceptable excipient or carrier.

### Variants and Fragments

The following section relates to general features of all proteins and/or peptides (i.e. polypeptides), and in particular to variations, alterations, modifications, fragments or derivatisations of amino acid sequence. It will be understood that such variations, alterations, modifications fragments or derivatisations of proteins and/or peptides as are described herein are subject to the requirement that the proteins and/or peptides retain any further required activity or characteristic as may be specified in other sections of this disclosure.

Variants of proteins and/or peptides may be defined by particular levels of amino acid identity which are described in more detail in subsequent sections of this disclosure. Amino acid identity may be calculated using any suitable algorithm. For example the PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring 20 matrix (see Henikoff and Henikoff (1992) Proc. Natl. Acad. Sci. USA 89: 10915-10919²⁵) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787²⁶. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001. Alternatively, the UWGCG Package provides the BESTFIT program which can be used to calculate homology (for example used on its default settings) (Devereux et al (1984) Nucleic Acids Research 12, 387-395²⁷).

It will be understood that variants of proteins and/or peptides also includes substitution variants. Substitution variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

The amino acid sequence of proteins and/or peptides for use in the invention may be modified to include non-naturally occurring chemistries or to increase the stability and targeting specificity of the compound. When the proteins and/or peptides are produced by synthetic means, such amino acids may be introduced during production. The proteins and/or peptides may also be modified following either synthetic or recombinant production.

A number of side chain modifications are known in the art and may be made to the side chains of the proteins and/or peptides, subject to the proteins and/or peptides retaining any further required activity or characteristic as may be specified herein.

Variant proteins and/or peptides as described in this section are those for which the amino acid sequence varies from that in SEQ ID NOs: 1-5, but which retain one or more advantageous properties.

The variant sequences typically differ by at least 1, 2, 3, 5, 10, 20, 30, 40 or more mutations (which may be substitutions, deletions or insertions of amino acids). For example, from 1 to 10, 2 to 5, or 3 to 20 amino acid substitutions, deletions (giving rise to a fragment) or insertions may be made, provided the modified proteins and/or peptide retains its activity. The amino acid substitutions, deletions or insertions may be contiguous or non-contiguous.

Typically, proteins and/or peptides variants, such as variants of the single pass EV transmembrane protein, have more than about 50%, 55% or 65% identity, preferably at least 70%, at least 80%, at least 90% and particularly preferably at least 95%, at least 97% or at least 99% identity, with the relevant amino acid sequence, such as any one or more of SEQ ID NOs: 1-5. The identity of variants of the relevant amino acid sequence, such as any one or more of SEQ ID NOs: 1-5, may be measured over a region of at least 10, 20, 30, 40 or more contiguous amino acids of the sequence, or more preferably over the full length of the relevant amino acid sequence, excluding any signal sequence.

Typically, proteins and/or peptides fragments, such as fragments of the single pass EV transmembrane protein, comprise at least 10, at least 20, at least 30, at least 40, at least 50 or more contiguous amino acid residues from the relevant amino acid sequence, such as any one or more of SEQ ID NOs: 1-5.

### Release Systems

Two types of release systems, in particular, are envisaged for use with the present invention. The first release system may be a release system that can be activated to release the cargo molecule. The second release system is an endosomal escape moiety that may facilitate the release of an EV from an endosome, such as from a late endosome. However, as will be apparent to the skilled person, the presence of either release system may not be essential, for example when the therapeutic effect of an EV arises from the interaction between a cargo molecule on the surface of an EV interacting with a moiety on the outer surface of a cell.

The first release system may be a small molecule induced, organic compound-based and/or polypeptide-based release system. Typically in a small-molecule induced release system, the presence of a particular small molecule will induce dimerization and formation of a covalent bond between the cargo molecule and the single pass EV transmembrane protein, for example between linkers fused to the cargo molecule and the single pass EV transmembrane protein. The absence or change in concentration of the same small molecule, or presence of a different small molecule, may result in the activation of the linker causing it to split into two discrete units, where the discrete units of the linker are not attached to each other. Typically, and organic compound or polypeptide of a release system will be a linker forming a covalent link between the single pass EV transmembrane protein and the cargo molecule. Again, the linker may be activated to split into at least two discrete units, wherein the discrete units of the linker are not attached to each other.

The first release system may comprise a reversible protein-protein interaction modules, such as one based on an aptamer-aptamer binding protein interaction or a nanobody interaction. For example, where one protein is fused to the cargo molecule and the other protein fused to the single pass EV transmembrane protein. When brought together, the two proteins may interact with each other in a reversible manner. This interaction may be though a reversible covalent bond, or through a reversible non-covalent interaction, such as one or more of Van der Waals forces, hydrophobic effects or ionic interactions. Upon activation, the interaction between the cargo molecule and the single pass EV transmembrane protein may be reduced such that the cargo molecule is released.

Thus, when the release system is activated, the cargo molecule is released from the membrane bound single pass EV transmembrane protein, and hence released from the membrane of the EV. If the cargo molecule is attached to the outer surface of the EV, then activating the linker results in the cargo molecule being released from its association with the EV. Suitable activatable linkers will be apparent to the skilled person from the prior art. For example, a linker may be activated a specific wavelength or light, or due to a change in pH such as due to the acidification of endosomes, or to the presence or absence of a specific small molecule.

When the release system is a polypeptide-based system it may be selected from the group comprising various releasable polypeptide interaction systems which may be activated or triggered without the need for exogenous stimuli (i.e. the release systems are typically triggered by endogenous activity within a cell or an EV, or essentially within any biological system), for instance a cis-cleaving polypeptide-based release system (e.g. based on inteins), a nuclear localization signal (NLS)—NLS binding protein (NLSBP)-based release system or release systems based on other protein domains. A monomeric light-induced cleavage-based release system may be utilized, where only a very short burst of light is utilized to start an endogenous proteolytic cleavage of a monomeric protein domain and release the cargo molecule.

The cis-cleaving or “self cleaving” sequence may be selected from inteins, light induced monomeric or dimeric release domains such as Kaede, KikGR, EosFP, tdEosFP, mEos2, PSmOrange, the GFP-like Dendra proteins Dendra and Dendra2, CRY2-CIBN, etc. Protease cleavage sites may also be incorporated into the fusion proteins for spontaneous release, etc., depending on the desired functionality of the fusion polypeptide. In the case of nucleic acid cargos specific nucleic acid cleaving domains may be included. Non-limiting examples of nucleic acid cleaving domains include endonucleases such as Cas6, Cas13, engineered PUF nucleases, site specific RNA nucleases etc. The inclusion of release domains is highly advantageous because they enable release of particular parts or domains from the original fusion polypeptide. This is particularly advantageous when the release of parts of the fusion polypeptide would increase bioactive delivery of the cargo and/or when a particular function of the fusion polypeptide works better when part of a smaller construct. The term “self-cleaving protein” can mean a naturally occurring protein that excises itself from a native host protein through self-cleavage. An intein, or PTTG1IP (in particular the sequence of SEQ ID NO: 5) are suitable examples of cis-cleaving sequences. It is to be appreciated that certain modifications may be desirable to provide a protein that has self-cleaving capability only (i.e., no self-splicing). As suitable example of a protein capable of only self-cleavage (and no splicing) is AI-CM. The term “self-splicing protein” can mean a naturally occurring protein that excises itself from a native host protein through self-splicing and ligation of their flanking peptide bonds. A suitable example of a self-splicing protein is an intein. The terms “mini-intein” or “delta-intein” are used interchangeably herein and can be understood to relate to a modified intein, preferably from parent RecA, and lacking the endonuclease domain. The term “fast-cleaving intein” can be understood to relate to an intein or mini-intein that has been modified at the +C-extein position and/or —N-extein position so that the cleavage rate of said intein is quicker/faster than the original RecA, intein, or mini-intein. The term “slow-cleaving intein” can be understood to relate to an intein or mini-intein that has been modified at the +C-extein position and/or —N-extein position so that the cleavage rate of said intein is slower than the original RecA, intein, or mini-intein. An example of a slow-cleaving intein is ΔI-CM. The cis-cleaving sequence may be a self-cleaving protein, for example an intein. The intein may be a slow-cleaving or a fast-cleaving intein. The intein may be a mini-intein, such as a mini-intein that has been modified to optimise the cleavage rate. The intein may be a delta-intein-CM. Thus, cis-cleaving sequences (or self-cleaving) in accordance with the disclosure may include, but are not limited to, the following:

Inteins, mini-inteins, delta inteins and certain variants, mutations and domains thereof having a desired functionality (including, but not limited to self-cleaving instead of splicing), such as a mini-intein modified to optimise the cleavage rate. For example, a mini-intein having C1A, D24G, V67L and/or D150G substitution in the N-terminal portion (or an appropriate—C-extein position. For example, splicing is enabled by the +1 position of the intein, wherein the +1 is Cys. Substitution of Cys with Ala in ΔI-CM removes the splicing capability and supports cleavage only. It will be appreciated that other mutations, substitutions, and the like may also similarly work. As such the substitution hereinbefore mentioned, while exemplary, is not limiting in any way and may also differ from intein to intein.

In certain instances, one may opt to utilize slow-cleaving inteins. A slow-cleaving system may be preferable when more time is required to ensure efficient loading of an EV with the desired cargo. Slow-cleaving inteins may include, but are not limited to, the following: mini-inteins, delta inteins, delta-intein-CM and a mini-intein and certain variants, mutations and domains thereof having a desired functionality (such as, but not limited to cleavage action instead of splicing and cleavage rate). In a preferred embodiment, the slow-cleaving cis-cleaving release system is based on an intein system, wherein the C-terminal portion of the intein may comprise the amino acid sequences Val-Val-Val-His-Asn, more preferably wherein the C-terminal portion of the intein is modified to comprise Val-Val-Val-His-Asn-Gly. Certain modifications at the +1 C-extein position, have been observed to slow the cleavage rate (i.e., are slow-cleaving). Alternatively, in certain instances, one may opt to utilize a fast-cleaving cis-cleaving release system (such as a fast-cleaving cis-cleaving intein). A fast-cleaving system may be preferable when EVs need to be harvested quickly. In a preferred embodiment, the fast-cleaving cis-cleaving release system is based on an intein system, wherein the C-terminal portion of the intein may comprise the amino acid sequences Val-Val-Val-His-Asn or Val-Val-Val-His-Asn-Cys. Certain modifications at the +1 C-extein position, such as the abovementioned example, have been observed to speed up the cleavage rate (i.e., are fast-cleaving).

The polypeptide-based release system may be present in multiple copies in series, such as 1, 2, 3, 4, or 5 copies, which may increase the effectiveness of the release system. The invention also relates to the release system per se and uses of the release system per se. For example, it would be apparent to the skilled person that a fusion protein comprising one or more copy of SEQ ID NO: 5, or a biologically active variant or fragment thereof, could be used to facilitate the separation of one or more fusion components of any fusion protein. The second release system may facilitate the release of an EV that has been taken up into the cell, for example by endocytosis. Endocytosis describes the physiological uptake of extracellular materials by cells through their encapsulation in vesicular compartments termed endosomes. Thus, when taken up into cells by endocytosis, an EV may be encapsulated in an endosome and it may be desirable to facilitate the release of an EV from an endosome. The skilled person would recognize that a number of approaches could be combined with the present invention to facilitate release of an EV from an endosome, such as a molecule that enhances release of an EV from an endosome. Such a molecule may be co-administered with the EV. Alternatively, or in addition, the EV may be modified to comprise such a molecule, for example, where the molecule that enhances release of an EV from an endosome is linked to an EV membrane-bound moiety, for example cholesterol, or a protein and/or peptide such as the single pass EV transmembrane protein. The molecule that enhances release of an EV from an endosome may be a pH-sensitive membrane-perturbing molecule. The molecule that enhances release of an EV from an endosome may be a molecule that binds to protons such as chloroquine, or variants thereof such as proton binding variants and hydroxychloroquine. The molecule that enhances release of an EV from an endosome may alternatively be fusogenic protein such as VSVG or a cationic peptide or polymer. Thus, the molecule that enhances release of the EV from endosomes may be any suitable endosomolytic compound.

Other endosomal escape peptides may be used in combination with the present invention, such as one or more endosomal escape peptide selected from HIV TAT PDT (peptide/protein transduction domain), KALA, GALA and INF-7 (derived from the N-terminal domain of influenza virus hemagglutinin HA-2 subunit), endosomal escape moieties that act by causing membrane fusion such as Diphtheria toxin T domain, proton sponge type endosomal escape moieties such as lipids with histidine or imidazole moieties and cell penetrating peptides (CPPs) and other moieties that enable endosomal escape by acting to puncture membranes. CPPs are typically less than 50 amino acids but may also be longer, are typically highly cationic and rich in arginine and/or lysine amino acids and have the ability to gain access to the interior of virtually any cell type, exemplary CPPs may be transportan, transportan 10, penetratin, MTS, VP22, CADY peptides, MAP, KALA, PpTG20, proline-rich peptides, MPG peptides, PepFect peptides, Pep-1, L-oligomers, calcitoninpeptides, arginine-rich CPPs such as poly-Arg, tat and combinations thereof).

### Compositions and Therapeutic Applications

Compositions of the invention comprise at least an EV as set out in other sections of this disclosure. Thus, the compositions of the invention may be used for targeted delivery of a cargo molecule. In addition, the compositions of the invention may be used as a research tool, a diagnostic tool, an imaging tool, biological reference material, an experimental control and/or an experimental standard.

Compositions comprising EVs loaded with a cargo molecule may be useful for vaccination, treating immune-privileged sites such as the eye, delivering a cargo molecule across the blood brain barrier, treating acute conditions such as severe combined immunodeficiency and treating chronic conditions such as myotonic dystrophy. The invention provides a simple, robust and efficient way to load a cargo molecule such as RNA-based, protein-based, gene therapy-based and/or gene editing-based drugs onto EVs to produce the compositions of the invention.

The use of EV compositions to deliver a cargo molecule offers a number of advantages over conventional means of delivering cargo molecules. For example, when a cargo molecule is delivered using EV compositions, it may be protected from degradation and may be more stable; a cargo molecule may be delivered to a target tissue, such as a specific type of cancer, more efficiently and/or more specifically than if not associated with an EV. In addition, the cargo molecule may be less likely to elicit an immune response when contained within EVs as it is not freely available for detection by immune cells and/or binding to antibodies. Other potential advantages of the use of EVs to deliver cargo molecules include avoiding drug resistance, such as the upregulation of drug transporters such as ABC-transporters, rapid tissue internalisation, single particle uptake, wide therapeutic index, broad biodistribution and good bioavailability.

The EV composition of the invention may be loaded with any cargo molecule that has utility in the treatment and/or prevention of a condition, disease or disorder. The cargo molecule to be loaded into the EV is chosen on the basis of the desired effect on the cell and/or tissue into which it is intended to be delivered and the mechanism by which that effect is to be carried out. A single cargo molecule may be incorporated into the EV. Alternatively, more than one cargo molecule may be incorporated into the EV. The more than one cargo molecules may act on the same or different targets to bring about their therapeutic and/or preventative effect.

The cargo molecule loaded into the composition may be a cargo molecule that is not used to generate an immune response. The cargo molecule may be selected to provide a therapeutic benefit itself, and is not intended to be used to generate an immune response against the cargo molecule. The cargo molecule to be incorporated into the EV composition may be useful, for example, in the prophylaxis and/or treatment and/or alleviation of a variety of diseases and conditions, typically via the delivery of essentially any type of drug cargo, such as for instance mRNA, antisense or splice-switching oligonucleotides, siRNA, pDNA, peptides, proteins, antibodies, antibody-drug conjugates, gene editing technology such as CRISPR-Cas9, TALENs, meganucleases, or vesicle-based cargos such as viruses (e.g. AAVs, lentiviruses, etc.) or EVs (exosomes, microvesicles and the like). Non-limiting examples of diseases and conditions that are suitable targets for treatment using the EV composition herein include the following non-limiting examples: Crohn's disease, ulcerative colitis, ankylosing spondylitis, rheumatoid arthritis, multiple sclerosis, systemic lupus erythematosus, sarcoidosis, idiopathic pulmonary fibrosis, psoriasis, tumour necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, Guillain-Barre syndrome, acute myocardial infarction, ARDS, sepsis, meningitis, encephalitis, liver failure, non-alcoholic steatohepatitis (NASH), non-alcoholic fatty liver disease (NAFLD), kidney failure, heart failure or any acute or chronic organ failure and the associated underlying aetiology, graft-vs-host disease, Duchenne muscular dystrophy and other muscular dystrophies, In-born errors of metabolism including: Disorders of carbohydrate metabolism e.g., G6PD deficiency galactosemia, hereditary fructose intolerance, fructose 1,6-diphosphatase deficiency and the glycogen storage diseases, Disorders of organic acid metabolism (organic acidurias) such as alkaptonuria, 2-hydroxyglutaric acidurias, methylmalonic or propionic acidemia, multiple carboxylase deficiency, Disorders of amino acid metabolism such as phenylketonuria, maple syrup urine disease, glutaric acidemia type 1, Aminoacidopathies e.g., hereditary tyrosinemia, nonketotic hyperglycinemia, and homocystinuria, Hereditary tyrosinemia, Fanconi syndrome, Primary Lactic Acidoses e.g., pyruvate dehydrogenase, pyruvate carboxylase and cytochrome oxidase deficiencies, Disorders of fatty acid oxidation and mitochondrial metabolism such as short, medium, and long-chain acyl-CoA dehydrogenase deficiencies also known as Beta-oxidation defects, Reye's syndrome, Medium-chain acyl-coenzyme A dehydrogenase deficiency (MCADD.), MELAS, MERFF, pyruvate dehydrogenase deficiency, Disorders of porphyrin metabolism such as acute intermittent porphyria, Disorders of purine or pyrimidine metabolism such as Lesch-Nyhan syndrome, Disorders of steroid metabolism such as lipoid congenital adrenal hyperplasia, congenital adrenal hyperplasia, Disorders of mitochondrial function such as Kearns-Sayre syndrome, Disorders of peroxisomal function such as Zellweger syndrome and neonatal adrenoleukodystrophy, congenital adrenal hyperplasia or SmithLemli-Opitz, Menkes syndrome, neonatal hemochromatosis, Urea cycle disorders such as N-Acetylglutamate synthase deficiency, carbamoyl phosphate synthetase deficiency, ornithine transcarbamoylase deficiency, citrullinemia (deficiency of argininosuccinic acid synthase), argininosuccinic aciduria (deficiency of argininosuccinic acid lyase), argininemia (deficiency of arginase), hyperornithinemia, hyperammonemia, homocitrullinuria (HHH) syndrome (deficiency of the mitochondrial ornithine transporter), citrullinemia II (deficiency of citrin, an aspartate glutamate transporter), lysinuric protein intolerance (mutation in y+L amino acid transporter 1, orotic aciduria (deficiency in the enzyme uridine monophosphate synthase UMPS), all of the lysosomal storage diseases, for instance Alpha-mannosidosis, Betamannosidosis, Aspartylglucosaminuria, Cholesteryl Ester Storage Disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis Type I, GM1 Gangliosidosis Type II, GM1 Gangliosidosis Type III, GM2—Sandhoff disease, GM2—Tay-Sachs disease, GM2—Gangliosidosis, AB variant, Mucolipidosis II, Krabbe Disease, Lysosomal acid lipase deficiency, Metachromatic Leukodystrophy, MPS I—Hurler Syndrome, MPS I—Scheie Syndrome, MPS I Hurler-Scheie Syndrome, MPS II—Hunter Syndrome, MPS IIIA—Sanfilippo Syndrome Type A, MPS IIIB—Sanfilippo Syndrome Type B, MPS IIIB—Sanfilippo Syndrome Type C, MPS IIIB—Sanfilippo Syndrome Type D, MPS IV Morquio Type A, MPS IV—Morquio Type B, MPS IX—Hyaluronidase Deficiency, MPS VI—Maroteaux-Lamy, MPS VII—Sly Syndrome, Mucolipidosis I—Sialidosis, Mucolipidosis IIIC, Mucolipidosis Type IV, Mucopolysaccharidosis, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Neuronal Ceroid Lipofuscinosis T9, Neuronal Ceroid Lipofuscinosis T10, Niemann-Pick Disease Type A, Niemann-Pick Disease Type B, Niemann-Pick Disease Type C, Pompe Disease, Pycnodysostosis, Salla Disease, Schindler Disease and Wolman Disease, etc. cystic fibrosis, primary ciliary dyskinesia, pulmonary alveolar proteinosis, ARC syndrome, Rett syndrome, neurodegenerative diseases including Alzheimer's disease, Parkinson's disease, GBA associated Parkinson's disease, Huntington's disease and other trinucleotide repeat-related diseases, dementia, ALS, cancer-induced cachexia, anorexia, diabetes mellitus type 2, and various cancers. Virtually all types of cancer are relevant disease targets for the present invention, for instance, Acute lymphoblastic leukemia (ALL), Acute myeloid leukemia, Adrenocortical carcinoma, AIDS-related cancers, AIDS-related lymphoma, Anal cancer, Appendix cancer, Astrocytoma, cerebellar or cerebral, Basal-cell carcinoma, Bile duct cancer, Bladder cancer, Bone tumour, Brainstem glioma, Brain cancer, Brain tumour (cerebellar astrocytoma, cerebral astrocytoma/malignant glioma, ependymoma, medulloblastoma, supratentorial primitive neuroectodermal tumours, visual pathway and hypothalamic glioma), Breast cancer, Bronchial adenomas/carcinoids, Burkitt's lymphoma, Carcinoid tumour (childhood, gastrointestinal), Carcinoma of unknown primary, Central nervous system lymphoma, Cerebellar astrocytoma/Malignant glioma, Cervical cancer, Chronic lymphocytic leukemia, Chronic myelogenous leukemia, Chronic myeloproliferative disorders, Colon Cancer, Cutaneous T-cell lymphoma, Desmoplastic small round cell tumour, Endometrial cancer, Ependymoma, Esophageal cancer, Extracranial germ cell tumour, Extragonadal Germ cell tumour, Extrahepatic bile duct cancer, Eye Cancer (Intraocular melanoma, Retinoblastoma), Gallbladder cancer, Gastric (Stomach) cancer, Gastrointestinal Carcinoid Tumour, Gastrointestinal stromal tumour (GIST), Germ cell tumour (extracranial, extragonadal, or ovarian), Gestational trophoblastic tumour, Glioma (glioma of the brain stem, Cerebral Astrocytoma, Visual Pathway and Hypothalamic glioma), Gastric carcinoid, Hairy cell leukemia, Head and neck cancer, Heart cancer, Hepatocellular (liver) cancer, Hodgkin lymphoma, Hypopharyngeal cancer, Intraocular Melanoma, Islet Cell Carcinoma (Endocrine Pancreas), Kaposi sarcoma, Kidney cancer (renal cell cancer), Laryngeal Cancer, Leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia), hairy cell leukemia)), Lip and Oral Cavity Cancer, Liposarcoma, Liver Cancer (Primary), Lung Cancer (Non-Small Cell, Small Cell), Lymphomas, Burkitt lymphoma, cutaneous T-Cell lymphoma, Hodgkin lymphoma, Non-Hodgkin, Medulloblastoma, Merkel Cell Carcinoma, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic/Myeloproliferative Diseases, Myelogenous Leukemia, Chronic Myeloid Leukemia (Acute, Chronic), Myeloma, Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumour), Ovarian germ cell tumour, Ovarian low malignant potential tumour, Pancreatic cancer, Pancreatic islet cell cancer, Parathyroid cancer, Penile cancer, Pharyngeal cancer, Pheochromocytoma, Pineal astrocytoma, Pineal germinoma, Pineoblastoma and supratentorial primitive neuroectodermal tumours, Pituitary adenoma, Pleuropulmonary blastoma, Prostate cancer, Rectal cancer, Renal cell carcinoma (kidney cancer), Retinoblastoma, Rhabdomyosarcoma, Salivary gland cancer, Sarcoma (Ewing family of tumours sarcoma, Kaposi sarcoma, soft tissue sarcoma, uterine sarcoma), Sezary syndrome, Skin cancer (nonmelanoma, melanoma), Small intestine cancer, Squamous cell, Squamous neck cancer, Stomach cancer, Supratentorial primitive neuroectodermal tumour, Testicular cancer, Throat cancer, Thymoma and Thymic carcinoma, Thyroid cancer, Transitional cell cancer of the renal pelvis and ureter, Urethral cancer, Uterine cancer, Uterine sarcoma, Vaginal cancer, Vulvar cancer, Waldenstrom macroglobulinemia, Wilms' tumour, genetic disorders, CNS conditions, neuro-degenerative disorders, liver disorders, heart disorders, Phenylketonuria, heart failure and/or ALS.

In a preferred embodiment, the disease or condition may be selected from Alzheimer's disease, autoimmune conditions, cancer, cardiovascular disease, cystic fibrosis, Duchenne muscular dystrophy, haemophilia, Huntington's disease, lysosomal storage disease, liver disorders, macular degeneration, myotonic dystrophy, neuromuscular disease, Parkinson's disease, sepsis, spinal muscular atrophy or stroke, genetic disorders, CNS conditions, neuro-degenerative disorders, heart disorders, Phenylketonuria, heart failure or ALS.

Thus, provided is a composition comprising an EV according to the present invention and at least one pharmaceutically acceptable excipient, for use in a method of therapy in a subject. Also provided is a composition comprising an EV according to the present invention and at least one pharmaceutically acceptable excipient, for use in a method of treating and/or preventing at least one of the therapeutic indications set out above.

Also provided is the use of a composition comprising an EV according to the present invention and at least one pharmaceutically acceptable excipient for the manufacture of a medicament for therapy. Also provided is the use of a composition comprising an EV according to the present invention and at least one pharmaceutically acceptable excipient, for the manufacture of a medicament for treating and/or preventing at least one of the therapeutic indications set out above.

Also provided is a method of treatment comprising providing an EV according to the present invention and at least one pharmaceutically acceptable excipient to a patient in need thereof. Also provided is a method of treatment and/or prevention comprising providing an EV according to the present invention and at least one pharmaceutically acceptable excipient to a patient in need thereof, wherein at least one of the therapeutic indications set out above is treated and/or prevented.

The use in medicine or method of treatment (collectively “the treatment”) may be by delivery of any kind of cargo according to the invention. For instance the treatment may be by delivery of functional proteins as protein replacement therapy, delivery of mRNA encoding for functional proteins to also act as a protein replacement therapy. Such a protein replacement therapy may, for instance, be an enzyme replacement therapy (ERT) for diseases caused by inborn errors in metabolism such as Phenylketonuria, urea cycle disorders, or lysosomal storage disorders. The treatment may be by delivery of: gene silencing RNAs, splice switching RNAs, or CRISPR-Cas9 for gene editing. The treatment may be gene therapy by delivery of plasmid DNA, mini-circles or viral gene therapies such as AAVs or lentiviruses. The treatment may be by presentation of an antigen or neoantigen for immunotherapy, in effect acting as a vaccine to induce an immune response. For instance the EV may act by delivery and/or presentation of a tumour antigen for cancer immunotherapy, or viral, bacterial or fungal antigens for immunization against pathogens. The treatment may be by delivery of small molecules, antibodies and antibody-drug conjugates capable of mediating a therapeutic effect once delivered into a cell or the extracellular matrix. In one embodiment, the treatment or therapy may be effected by the EVs comprising more than one type of therapeutic cargo, i.e. the therapeutic cargo may be a mixture of protein, nucleic acid, virus, viral genome, antigen and/or small molecule.

### Delivery/Administration

The EVs of the invention may be administered by any suitable means. Administration to a human or animal subject may be selected from parenteral, intramuscular, intracerebral, intravascular (including intravenous), subcutaneous, intranasal, intracardiac, intracerebroventricular, intraperitoneal or transdermal administration. Typically, the method of delivery is by injection. Preferably the injection is intramuscular or intravascular (e.g. intravenous). A physician will be able to determine the required route of administration for each particular patient.

The EVs are preferably delivered as a composition. The composition may be formulated for any suitable means of administration, including parenteral, intramuscular, intracerebral, intravascular (including intravenous), intracardiac, intracerebroventricular, intraperitoneal, subcutaneous, intranasal or transdermal administration. Compositions for parenteral administration may include sterile aqueous solutions which may also contain buffers, diluents and other suitable additives. The EVs of the invention may be formulated in a pharmaceutical composition, which may include pharmaceutically acceptable carriers, thickeners, diluents, buffers, preservatives, and other pharmaceutically acceptable carriers or excipients and the like in addition to the EVs.

A “pharmaceutically acceptable carrier” (excipient) is a pharmaceutically acceptable solvent, suspending agent or any other pharmacologically inert vehicle for delivering one or more nucleic acids to a subject. Typical pharmaceutically acceptable carriers include, but are not limited to, binding agents (e.g. pregelatinised maize starch, polyvinylpyrrolidone or hydroxypropyl methylcellulose, etc); fillers (e.g. lactose and other sugars, microcrystalline cellulose, pectin, gelatin, calcium sulfate, ethyl cellulose, polyacrylates or calcium hydrogen phosphate, etc); lubricants (e.g. magnesium stearate, talc, silica, colloidal silicon dioxide, stearic acid, metallic stearates, hydrogenated vegetable oils, corn starch, polyethylene glycols, sodium benzoate, sodium acetate, etc); disintegrates (e.g. starch, sodium starch glycolate, etc); or wetting agents (e.g. sodium lauryl sulphate, etc).

The term “excipient” or “carrier” refers to an inert substance added to a pharmaceutical composition to further facilitate administration of a compound. The terms encompass any of the agents approved by a regulatory agency such as the FDA or EMEA or listed in the U.S. Pharmacopeia for use in animals, including humans, as well as any carrier or diluent that does not cause significant irritation to a subject and does not abrogate the biological activity and properties of the therapeutic cargo. Included are excipients and carriers that are useful in preparing a pharmaceutical composition and are generally safe and non-toxic.

Further exemplary excipients include degradation or loss of activity stabiliser excipients such as proteins such as human serum albumin, polyols such as glycerol, sorbitol and erythritol, amino acids such as arginine, aspartic acid, glutamic acid, lysine, proline, glycine, histidine and methionine, polymers such as polyvinylpyrrolidone and hydroxypropyl cellulose, surfactants such as polysorbate 80, polysorbate 20 and pluronicF68, antioxidants such as ascorbic acid and alpha-tocopherol (vitamin E), buffers such as acetate, succinate, citrate, phosphate, histidine, tris(hydroxymethyl)aminomethane (TRIS), metal ion/chelators such as Ca2+, Zn2+ and EDTA, Cyclodextrin based such as hydroxypropyl B-cyclodextrin and others such as polyanions and salts, stabilisers or bulking agents such as lactose, trehalose, dextrose, sucrose, sorbitol, glycerol, albumin, gelatin, mannitol and dextran, or preservatives such as benzyl alcohol, m-cresol, phenol, 2-phenoxyethanol.

The compositions provided herein may additionally contain other adjunct components conventionally found in pharmaceutical compositions. Thus, for example, the compositions may contain additional compatible pharmaceutically-active materials or may contain additional materials useful in physically formulating various dosage forms of the composition of present invention, such as dyes, flavouring agents, preservatives, antioxidants, opacifiers, thickening agents and stabilizers. However, such materials, when added, should not unduly interfere with the biological activities of the components of the compositions provided herein.

A therapeutically effective amount of composition is administered. The dose may be determined according to various parameters, especially according to the severity of the condition, age, and weight of the patient to be treated; the route of administration; and the required regimen. A physician will be able to determine the required route of administration and dosage for any particular patient. Optimum dosages may vary depending on the relative potency of individual EVs, and can generally be estimated based on EC50s found to be effective in vitro and in in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the specific EV, the age, weight and condition of the subject to be treated, the severity of the disease and the frequency and route of administration. Different dosages of the EV may be administered depending on whether administration is by intramuscular injection or systemic (intravenous or subcutaneous) injection. Preferably, the dose of a single intramuscular injection is in the range of about 5 to 20 pg. Preferably, the dose of single or multiple systemic injections is in the range of 10 to 100 mg/kg of body weight.

It will be clear to the skilled artisan that when describing medical and scientific uses and applications of the EVs, the present invention normally relates to a plurality of EVs, i.e. a population of EVs which may comprise thousands, millions, billions or even trillions of EVs. EVs may be present in concentrations such as about 10⁵, 10⁸, 10¹⁰, 10¹¹, 10¹², 10¹³, 10¹⁴, 10¹⁵, 10¹⁸, 10²⁵, 10³⁰ EVs (often termed “particles”) per unit of volume (for instance per ml), or any other number larger, smaller or anywhere in between. In the same vein, the term “population”, which may e.g. relate to an EV comprising certain cargo molecules shall be understood to encompass a plurality of entities constituting such a population. In other words, individual EVs when present in a plurality constitute an EV population. Thus, naturally, the present invention pertains both to individual EVs and populations comprising EVs, as will be clear to the skilled person. The dosages of EVs when applied in vivo may naturally vary considerably depending on the disease to be treated, the administration route, the activity and effects of the cargo molecule of interest, any targeting moieties present on the EVs, the pharmaceutical formulation, etc.

The composition according to the present invention may be formulated by any known method of formulation including but not limited to
Oral formulations—Tablet, Capsule, Sustained release, liquid
Intravenous Formulations
Parenteral Formulations
Topical Formulations—cutaneous administration: cream, ointment, gel, paste, powder
Modified release Formulations—sustained release formulation
Liquid or lyophilized formulations
Nebulized formulations

Additional routes of administration by which the EVs/compositions of the invention may be administered to a human or animal subject include auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracerebroventricular, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumour, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the EVs, the cargo molecule in question, or the EV population as such.

Due to EV clearance (and breakdown of any cargo molecule), the patient may have to be treated repeatedly, for example once or more daily, weekly, monthly or yearly. Persons of ordinary skill in the art can easily estimate repetition rates for dosing based on measured residence times and concentrations of the EV in bodily fluids or tissues. Following successful treatment, it may be desirable to have the patient undergo maintenance therapy, wherein the EV is administered in maintenance doses, ranging from 0.01 mg/kg to 100 mg per kg of body weight, once or more daily, to once every 20 years. Thus, it is envisaged that any dosage regime would be applicable to the engineered EVs of the invention. The dosage regime chosen will depend on the cargo being delivered by the EVs and the disease to be treated and any additional therapies being administered which will be determined by the skilled physician. It is envisaged that the EVs of the present invention will be administered multiple times, i.e. more than 1 time but normally more than 2 times or potentially for chronic, long-term treatment (i.e. administered tens to hundreds to thousands of times). Preferably, if the cargo is an antigen that is being administered as a vaccine, the immunization schedule will involve two or more administrations of the polypeptide, spread out over several weeks. Similarly, if the cargo is e.g. an RNA agent such as an siRNA or mRNA or a protein such as an antibody or an enzyme or a transporter, the EVs comprising the cargo in question will likely be administered more than once, normally multiple times as part of a chronic treatment regimen.

A composition of the invention may be co-administered with one or more other agent. The one or more other agent may be administered separately to the composition of the invention, at substantially the same time as the composition of the invention, or as a single composition comprising the EV of the invention in combination with the one or more other agent. Thus, combination therapy comprising the EV of the invention and one or more other agent is envisaged. The one or more other agent may be loaded into the EV, for example it may be encapsulated inside the EV or bound to the surface of the EV. By way of example, the composition of the invention may be co-administered with a cell-penetrating peptide (CCP) to assist intracellular delivery and/or cell-specific targeting.

The EV may be particularly suited to the delivery of cargo molecules in vitro and/or in situ. When delivered in situ, the EVs are produced in the patient tissue by EV host cells (i.e. producer cells) present in the patient. The host cells present in the patient may have been produced by any suitable methods, such as a method described herein, and introduced into the patent.

Purification and/or Identification of an EV

The single pass EV transmembrane protein and/or EV composition comprising the single pass EV transmembrane protein may be used to purify and/or to identify an EV. For example, a molecule that binds to the single pass EV transmembrane protein and/or the moiety on the surface of the EV fused to the single pass EV transmembrane protein, may be used to capture an EV in bulk solution through its direct and/or indirect interaction with the EV restricted single pass EV transmembrane protein, in accordance with methods known in the prior art.

For example, the molecule that binds to the single pass EV transmembrane protein and/or the moiety on the surface of the EV fused to the single pass EV transmembrane protein may be immobilised on a solid support to capture the EV composition of the invention on the solid support, thus immobilising the EV composition on the same solid support. Thus, the EV composition itself may be immobilised on a solid support. Any suitable solid support may be used, such as a bead which may be a magnetic bead, or the surface of a 96-well plate. The captured/immobilised EV may then be eluted from the solid support, for example by altering the salt concentration, altering the pH, and/or washing with a fluid such as glycerol.

Any suitable molecule may be used to capture the EV, and may be appropriately selected from, for example, one or more of a peptide and/or protein, an antibody and/or antigen-binding variant or fragment thereof, a single chain variable fragment (scFv), a nanobody, a nucleic acid-binding protein and/or peptide, a RNA- and/or DNA-binding protein, a small molecule drug, a nucleic acid, a nucleic acid analogue, an unnatural nucleic acid, gRNA, miRNA, shRNA, siRNA, piRNA, PMO, DNA and a DNA plasmid.

The molecules used to capture an EV may comprise a single type of molecule, two different types of molecules, three different types of molecules, four different types of molecules, five different types of molecules, six different types of molecules, seven different types of molecules, eight different types of molecules, nine different types of molecules, ten different types of molecules, or more than ten different types of molecules. In other words, the molecules used to capture an EV may be homogenous or heterogonous.

By capturing an EV in this way, it is possible to purify an EV from bulk solution. It is also possible to use the molecule that captures the EV as an in vitro and/or ex vivo research tool, diagnostic tool such as in an ELISA assay, imaging tool for example by conjugating the molecule that captures the EV to a fluorophore, a biological reference material, an experimental control and/or an experimental standard.

Purification of EVs may be achieved by any method including but not limited to: techniques comprising liquid chromatography (LC), high-performance liquid chromatography (HPLC), bead-eluate chromatography, ionic exchange chromatography, spin filtration, tangential flow filtration (TFF), hollow fiber filtration, centrifugation, immunoprecipitation, flow field fractionation, dialysis, microfluidic-based separation, etc., or any combination thereof. In an advantageous embodiment, the purification of the EVs is carried out using a sequential combination of filtration (preferably ultrafiltration (UF) tangential flow filtration (TFF) or hollow fibre filtration) and affinity chromatography, optionally also including size exclusion LC or bead-eluate LC. Combining purification steps normally enhances the purity of the resulting samples and, in turn leads to superior therapeutic activity. Further, as compared to UC, which is routinely employed for purifying exosomes, sequential filtration-chromatography is considerably faster and possible to scale to higher manufacturing volumes, which is a significant drawback of the current UC methodology that dominates the prior art. Another advantageous purification method is TFF, which offers scalability and purity, and which may be combined with any other type of purification technique.

### Isolating EVs and Production of EV Compositions

Suitable cells for production of EVs will be apparent to the skilled person. Any EV-producing cell can be utilized. Suitable physiological fluids from which EVs can be isolated will also be apparent to the skilled person. EVs can be collected from a cell culture medium and/or a physiological fluid by any suitable method. EVs may be isolated from a suitable cell bank. Alternatively, EVs may be isolated form any autologous patient-derived, heterologous haplotype-matched or heterologous stem cells so to reduce or avoid the generation of an immune response in a patient to whom the EVs are delivered.

Typically, a preparation of EVs can be prepared from cell culture tissue supernatant or physiological fluid by centrifugation, filtration or combinations of these methods. For example, EVs can be prepared by differential centrifugation, that is low speed (<20,000 g) centrifugation to pellet larger particles followed by high speed (>100,000 g) centrifugation to pellet EVs, size filtration with appropriate filters (for example, a molecular weight cut-off filter for 1 MDa, 300 kDa, 100 kDa, 10 kDa or 1 kDa), gradient ultracentrifugation (for example, with sucrose gradient) or a combination of these methods. Isolated EVs may be further purified, concentrated and/or diluted as appropriate.

The isolated EVs may already represent, either in whole or in part, the EVs comprised in the compositions of the invention, for example due to the endogenous expression in the host cell of the single pass EV transmembrane protein, moiety on the surface of the EV and/or cargo molecule. Alternatively or in addition, isolated EVs may be manipulated to produce the EVs comprised in the compositions of the invention, for example through the addition of additional molecules.

Thus the EV composition of the invention may be produced by transforming, transfecting and/or transducing a host cell with a nucleic acid construct. The nucleic acid construct may comprise, for example, a plasmid expressing a single pass EV transmembrane protein fused to: (a) a moiety expressed on the surface of the EV; and/or (b) a cargo molecule. The nucleic acid construct is then expressed in the host cell allowing EVs to be obtained from the host cell in which the single pass EV transmembrane protein has been expressed endogenously. The nucleic acid construct may be transiently and/or stably introduced into the host cell. In one embodiment, stable introduction of the nucleic acid construct may be preferable.

Alternatively, or in addition, an EV may be obtained, for example from one or more host cell (i.e. producer cell), and one or more component of the invention introduced exogenously, for example by electroporation and/or transfection. For example, EVs expressing a single pass EV transmembrane protein fused to a moiety on the surface of the EV may be produced endogenously by the host cell, and then a nucleic acid cargo molecule produced by cell-free syntheses electroporated and/or transfected into the EV.

The host cell may be cultured in a bioreactor, such as a hollow-fibre bioreactor, to produce a conditioned media and the EVs obtained from the conditioned media. The use of a bioreactor to culture the host cell may allow for the generation of a large quantities of EV-containing conditioned media. Optionally, the EVs may be purified from the conditioned media using one or more of liquid chromatography, tangential flow filtration, ultracentrifugation (UC), density-gradient separation, antibody precipitation, polymer precipitation and the purification methodology set out in the previous section.

Thus, a further advantage of the present invention is that the EV composition may be produced with minimal steps. For example, no further substantive processing of the EV may be necessary following isolation of the EV from a host cell, for example if the host cell produces an EV comprising a single pass EV transmembrane protein fused to a moiety on the surface of the EV and/or a cargo molecule. Alternatively, it may be possible to load an isolated EV with, for example a cargo molecule, in a single step, for example by transfecting an isolated EV with a cargo molecule. In such a way, the invention also provides a platform technology in which a desired EV composition can be quickly, efficiently and robustly produced by selecting appropriate components from a previously generated EV producing host cells and/or EVs, and additional components such as desired cargo molecules. The EV producing host cells, EVs and additional components such as desired cargo molecules may be selected from a previously generated library.

When a population of EVs is produced, at least 5%, at least 10%, at least 20%, at least 50%, at least 70%, at least 75%, at least 80%, at least 85%, at least 90%, and/or at least 95% of all EVs in the population may comprise at least one fusion protein construct.

### Exogenous Cargo Loading Strategies

EV cargo may be passively loaded into EVs or actively loaded by exogenous loading methods as described below. The cargo may be loaded passively into the EVs by the therapeutic cargo being present in the cytosol of the EV producing cells. Such passive loading applies, for instance, to nucleic acids, small molecules, viruses, soluble proteins or membrane proteins that are naturally loaded into the EVs. In certain embodiments the cargo is actively loaded into the EVs of the present invention. One form of active loading of cargos involves exogenous active loading which involves cargo being loaded using any known exogenous loading method including: electroporation, transfection with transfection reagents such a cationic transfection agents, lipofectamine, conjugation of the cargo to a membrane anchoring moiety such as a lipid or cholesterol tail or loading by means of a cell penetrating peptide (CPP), either in the form of a CPP-cargo conjugate or in the form of a CPP-cargo non-covalent complex or any combination of these methods. Again, this type of active loading may result in the therapeutic cargo being located on the inside of the EV, on the outside of the EV or located within the membrane of the EV. Any of the cargos as defined above may be loaded exogenously. Particularly preferred embodiments involve exogenous loading of nucleic acid or viral cargos by electroporation, CPP loading or co-incubation with a lipid tagged cargo.

### EXAMPLES

The invention is described in more detail below with reference to the following non-limiting Examples, which are intended to aid the comprehension of the invention.

### Example 1—Materials and Methods

### Plasmids

Plasmids pCAG-CD63-intein-Cre, pSpCas9(BB)-2A-Puro (PX459) and pCMV-VSVG plasmid (pMD2.G) were kind gifts from Samir EL Andaloussi (Karolinska Institutet, Sweden), Feng Zhang (Addgene plasmid #62988; https://www.addgene.org/62988/; RRID: Addgene_62988) and Didier Trono (Addgene plasmid #12259; https://www.addgene.org/12259/; RRID: Addgene_12259), respectively. Remaining constructs were cloned in house by standard recombinant DNA technology.

### Cell Culture

Human embryonic kidney (HEK) 293T cells were cultured at 37° C. with 5% C02 in Dulbecco's Modified Eagle Medium GlutaMAX (DMEM) (Gibco) supplemented with 10% foetal bovine serum (FBS) and 1% Antibiotic-Antimycotic (PSA: Penicillin, Streptomycin and Amphotericin B) (Thermo Fisher Scientific, Massachusetts, United States).

T47D, HeLa and B16 traffic light Cre reporter cells (30,000 cells per well) were seeded onto 96-well plates with complete culture medium (DMEM+10% FBS+1% PSA) and incubated at 37° C. with 5% CO₂. Next day, cells were treated with EVs in 65 μl of complete Opti-MEM (Thermo Fisher Scientific). 24h later, media was changed to culture medium (DMEM+10% FBS+1% PSA) and 24h after media change cells were analysed by flow cytometry.

HEK293T stoplight light Cas9 reporter cells¹² (30,000 cells per well) were seeded onto 96-well plates with complete medium and incubated at 37° C. under 5% C02. For experiments using sgRNA expressing cells, sgRNA was transfected 24h later. Cells were transfected with 45 ng of plasmid DNA/well using polyethylenimine (PEI) (Sigma-Aldrich) at a 4:1 ratio PEI:DNA in complete media. 24h later media was removed, and cells were treated with EVs in 65 μl of complete Opti-MEM (Thermo Fisher Scientific). For non-sgRNA expressing experiments, cells were treated 24h after seeding with EVs in 65 μl of complete Opti-MEM (Thermo Fisher Scientific). In both cases media was changed to complete medium 24h later and cells were analysed by flow cytometry 48h after EV transfer.

### EV Production

For in vitro experiments, at least 4 150 mm plates of HEK293T cells per condition were transfected with 35 pg DNA/plate. 24h later, media was replaced with Opti-MEM (Thermo Fisher Scientific) and 48h later, cell culture supernatant was collected for subsequent EV isolation.

For the in vivo experiment, 60 150 mm plates of HEK293T cells per condition were transfected with 35 pg DNA/plate. 24h later, media was replaced with Opti-MEM (Thermo Fisher Scientific) and 48h later, cell culture supernatant was collected for subsequent EV isolation.

### EV Isolation

HEK293T cells were transfected with different constructs as indicated. 24 hours later, the medium was replaced with opti-MEM (Thermo Fisher Scientific) and 48h later, cell culture supernatant was collected and centrifuged consecutively at 500 g and 2,000 g to remove cell debris and large particles, respectively. The media was then filtered through a 0.45 μm filter (STARLAB, Hamburg, Germany) and concentrated using a 10 kDa vivaflow tangential flow filtration (TFF) membrane (Sartorius, Gottingen, Germany) to 10-15 ml. Then, using 10 kDa Amicon spin filters (Merck Millipore, Massachusetts, United States) media was concentrated further to 1-2 ml by centrifugation at 3,500 g. Following this, concentrated media was subjected to size exclusion chromatography using HiPrep Sephacryl S-400 HR 16/60 column (GE Healthcare) connected to the AKTA prime system (GE Healthcare) and eluted at 0.5 ml/min flow rate using PBS as the eluent. EV and protein fractions were selected based on 280 nm absorbance. The yield of vesicles was measured using single-vesicle analysis (nanoFCM).

### Flow Cytometry

Cells were washed with PBS followed by 15 min incubation with 50 μl TrypLE Express (Thermo Fisher Scientific). Trypsin was inactivated using 100 μl FACS buffer (PBS containing 10% FBS) and cells were kept on ice until analysis. Cells were analysed on CytoFLEX LX flow cytometer (Beckman Coulter) and data analysis was performed using FlowJo software. Activated reporter cells were identified by GFP expression (measured at 488-525 nm).

### Single Vesicle Analysis

HEK293T cells were seeded on 12-well plates (100,000 cells per well) 24 h before transfection. The following day, cells were transfected with 700 ng of plasmid DNA/well using polyethylenimine (PEI) (Sigma-Aldrich, Missouri, United States) at a 4:1 ratio PEI:DNA in Opti-MEM (Thermo Fisher Scientific). Next day, media was replaced with Opti-MEM and 48h later conditioned media was collected. Before single-vesicle analysis, condition media was centrifuged first at 500 g to remove cells and subsequently at 2,000 g to remove large particles. After 0.22 μM filtering (STARLAB), conditioned media was analysed on a NanoAnalyzer N30 instrument (nanoFCM INC, Xiamen, China). 2,000-12,000 single particle events were counted for 1 min using light scattering and 488/24 nm blue laser set to 10 mW and 10% SS decay, at a sampling pressure of 1.0 kPa. Data were analysed using NanoFCM Professional Suite v1.8 software (manufacturer). EV size and concentration were determined by interpolation from a standard curve. Non-transfected HEK293T cells conditioned media was used as a fluorescence negative control to set the threshold for GFP positivity.

### N-Glycosylation Assays

PNGase F and Endonuclease H treatments were performed to assess the N-glycosylation status of PTTG1IP.

For both treatments, 20 pg protein lysate from HEK293T cells transfected with the experimental plasmid constructs was denatured for 10 min at 100° C. with 10× Glycoprotein Denaturing Buffer. For PNGase F, this was followed by incubation with 1 μL PNGase F (NEB), 2 μL 10% NP-40 (NEB) and 2 μL 10× GlycoBuffer 2 (NEB, Massachusetts, United States) buffer at 37° C. 1 h. For Endonuclease H treatment, after denaturation, samples were incubated with 1.5 μL EndoH and 2 μL 10× GlycoBuffer 3 (all NEB) buffer at 37° C. 1 h. Glycosylation status was determined as a shift in protein size by Western blot.

### Western Blot

Cells were lysed in 1x RIPA buffer (Thermo Fisher Scientific) containing 1× cOmplete protease inhibitor cocktail (Merck, Darmstadt, Germany). Acetone precipitation was used for EV samples, where cold (−20° C.) acetone at was added 4:1 and incubated for 24 h at −20° C. for subsequent centrifugation at 5,000 g 20 min. The resulting pellet was resuspended in 1×RIPA buffer (Thermo Fisher Scientific) containing 1× cOmplete protease inhibitor cocktail (Merck) and 10% NuPage sample reducing agent (Invitrogen). Protein concentration was determined by Bradford assay (Sigma-Aldrich) following manufacturer's instructions. 5-10 ug of total protein was heated at 70° C. for 10 min and then resolved on a 4-12% NuPAGE gel (Thermo Fisher Scientific) by SDS-PAGE. Gels were run at 150 V for 2h in 1× NuPAGE Tris-Acetate SDS Running Buffer (Thermo Fisher Scientific). Protein was transferred onto a PVDF membrane (Merck) at 60 V for 1 5 hr 30 min in 1× NuPAGE Transfer Buffer (Thermo Fisher Scientific). Membranes were blocked in Intercept (PBS) Blocking Buffer (Sigma-Aldrich) for 30 mins at room temperature and incubated in Blocking Buffer with primary antibodies at 4° C. overnight. Blots were washed with PBS supplemented with 0.1% Tween-20 (PBS-T) three times and incubated with secondary antibody for 1 hr at room temperature. Antibody information is 10 described in the Table below). Blots were imaged using a LI-COR Biosciences Odyssey Fc instrument (LI-COR, NE, USA).

Primary and secondary antibody information

| Application | Antibody | Host | ID | Manufacturer | Dilution |
|---|---|---|---|---|---|
| Primary | Anti-GFP | Rabbit | ab290/ | Abcam/ | 1:1,000 |
| | | | 338001 | Biolegend | |
| Primary | Anti-cMyc | Mouse | 626801 | Biolegend | 1:1,000 |
| Primary | Anti-CD63 | Mouse | Ab1289 | Abcam | 1:1,000 |
| Primary | Anti-bActin | Mouse | MA5-15739 | ThermoFisher | 1:2,000 |
| Primary | Anti-ALIX | Rabbit | ab186429 | Abcam | 1:2,000 |
| Secondary | Anti-Rabbit | Goat | 926-32211 | LI-COR | 1:5,000 |
| Secondary | Anti-Mouse | Goat | 926-32210 | LI-COR | 1:5,000 |

### In Vivo EV-Transfer

Mice were injected with 500,000 B16F10 Cre reporter cells/mouse 10 days before EV treatment. On day 10, isolated EVs were injected to recipient mice by intra-tumoural injection (1×10¹⁰ EVs/mice). 4 days later, tumours were harvested and Cre activity was analysed by immunohistochemistry (ICH) and PCR.

### Statistics

For the bioinformatics analysis, Kruskall-Wallis test was used to compare the medians of EV to cell abundance ratio of proteins with a given feature and that of all proteins in the dataset and determine if any of the medians were significantly different (P-value<0.05). To identify features that were significantly EV-enriched (P-value<0.05), Kolmogorov-Smirnov test followed by Benjamini-Yekuteli post hoc test was applied.

GraphPad Prism software (GraphPad, San Diego, California) was used to generate graphs and calculate standard error of the mean (SEM). Student's t test was performed to assess statistical differences between two groups. One-way ANOVA followed by Tukey's post hoc test was performed when analyzing more than 2 groups. Results were considered significant at a confidence of 95%, indicated on graphs with asterisks (* P<0.05).

### Example 2—in Silico Identification of EV-Loading Features

Protein features including post-translational modifications (PTMs), protein motifs, and protein domains such as ubiquitin-like modifications¹³ or late domain motifs¹⁴ have been shown to play a role in EV-protein sorting⁵. Therefore, to identify novel features that could lead to protein sorting to EVs it was decided to develop an in silico approach to analyze protein features enriched in EVs according to matched EV and producer cell proteomics datasets from three different cell lines: HEK293T, MSCs and adipocytes.

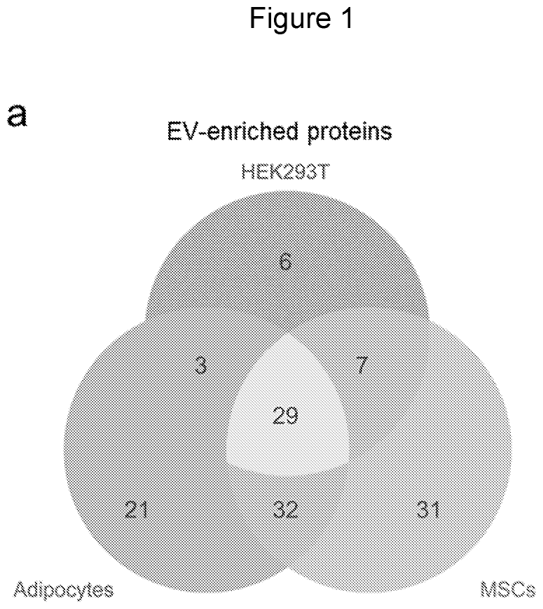

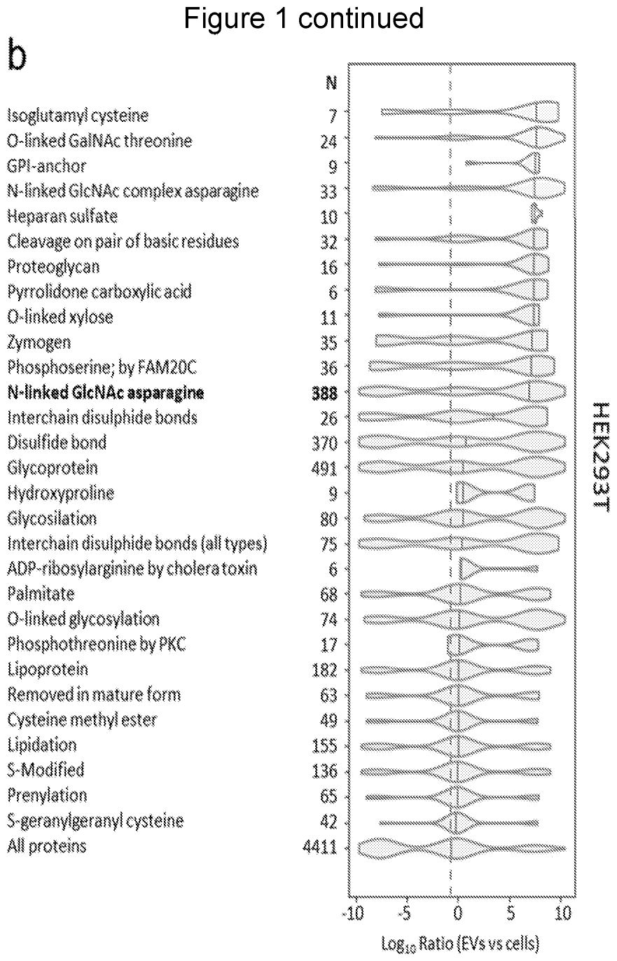

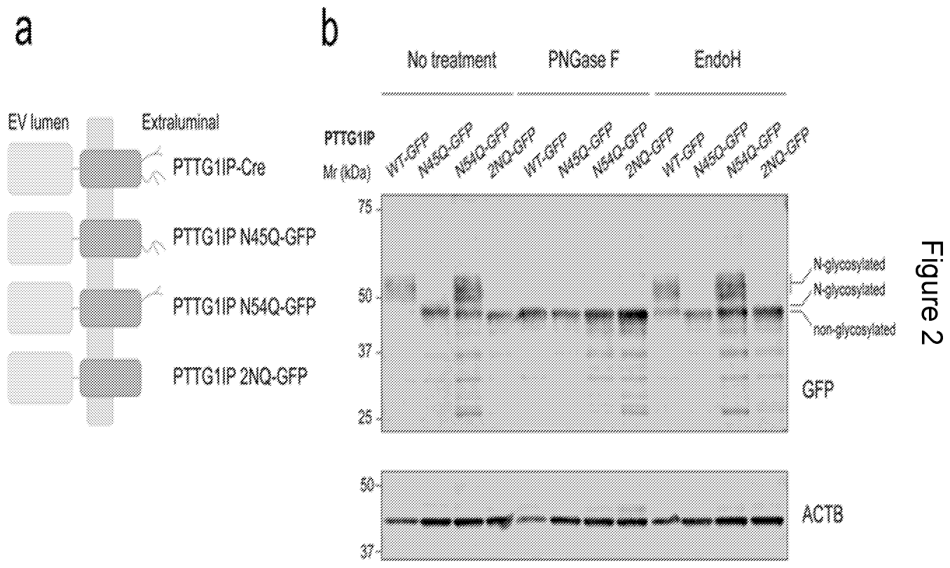

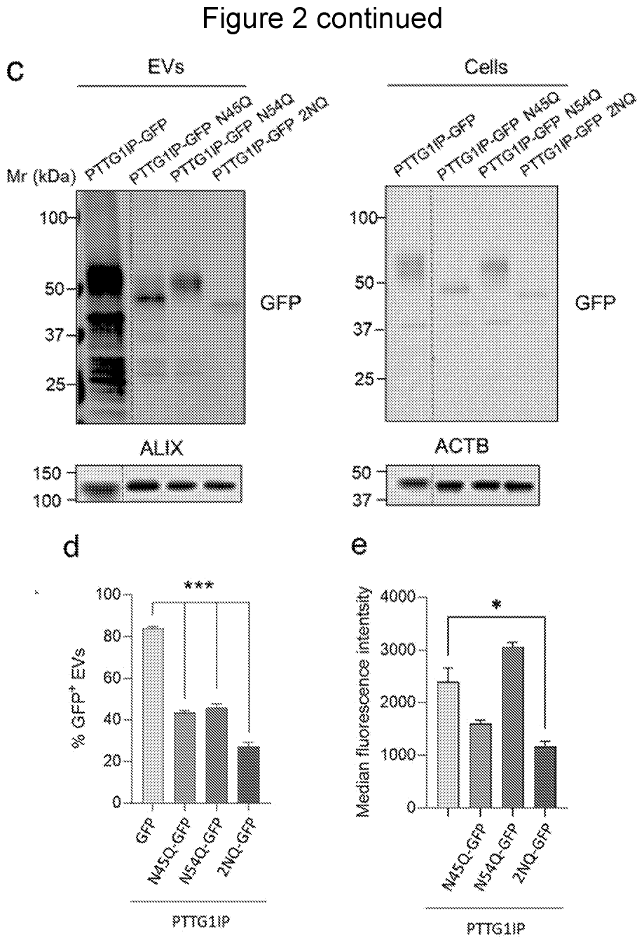

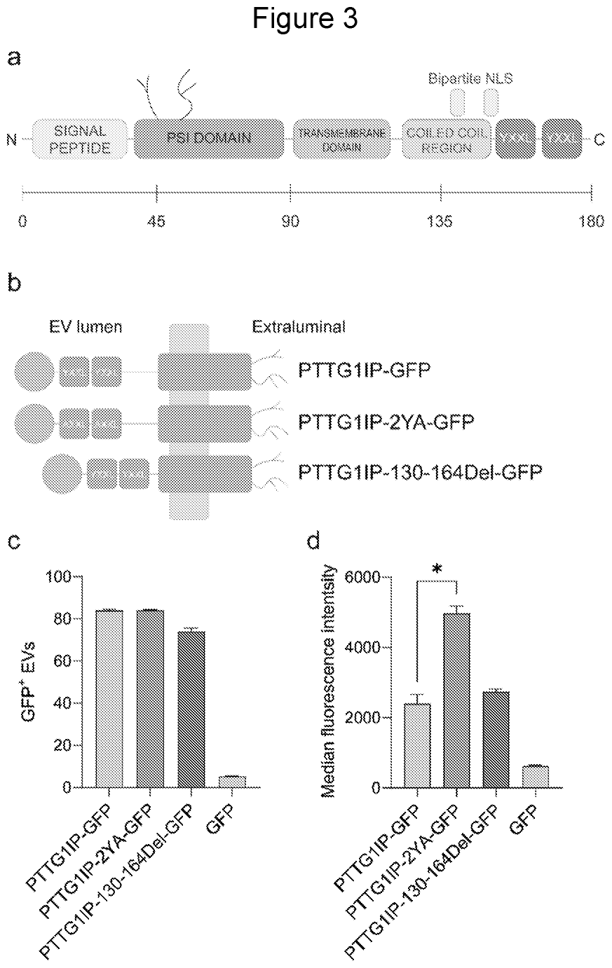

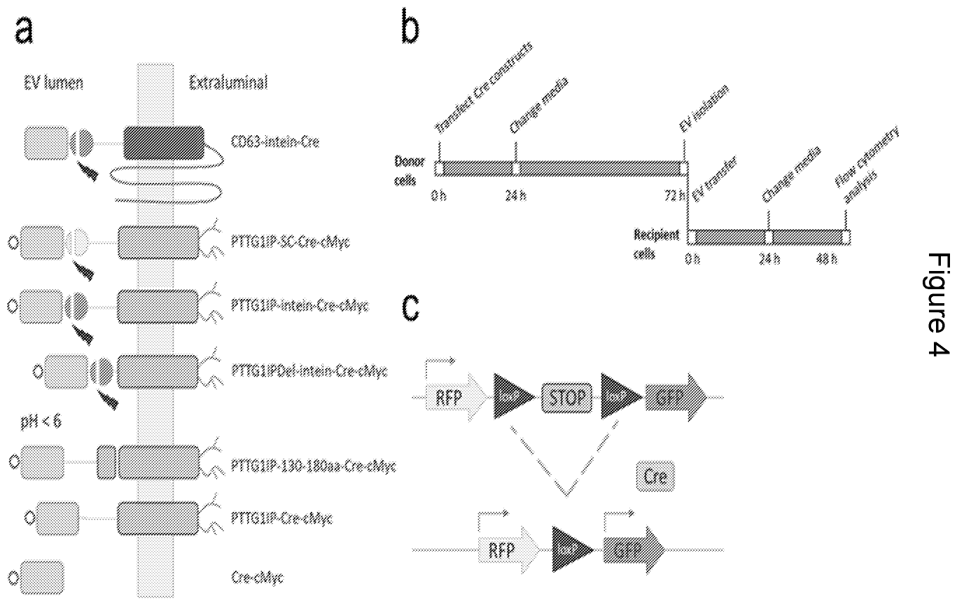

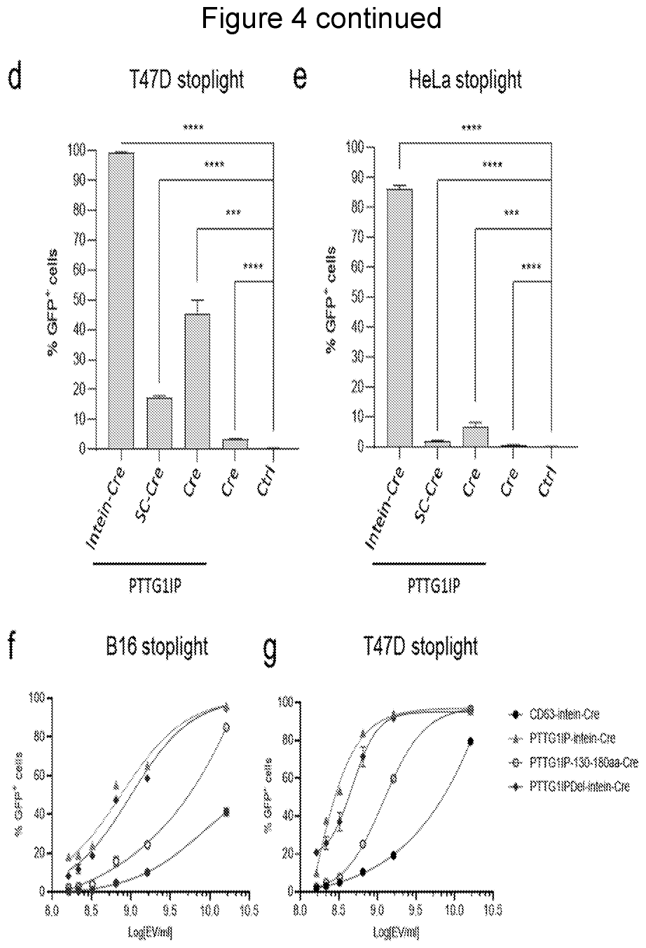

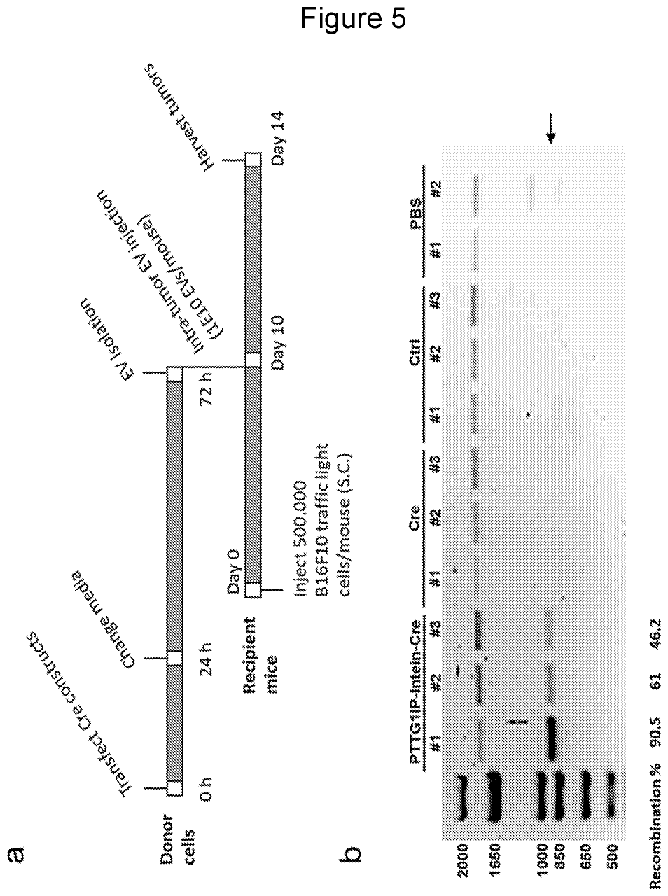

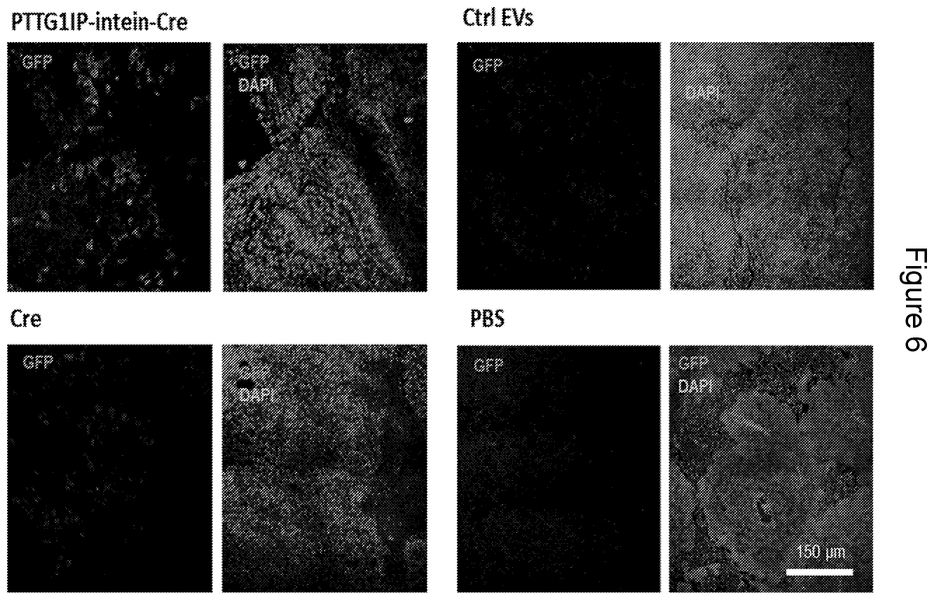

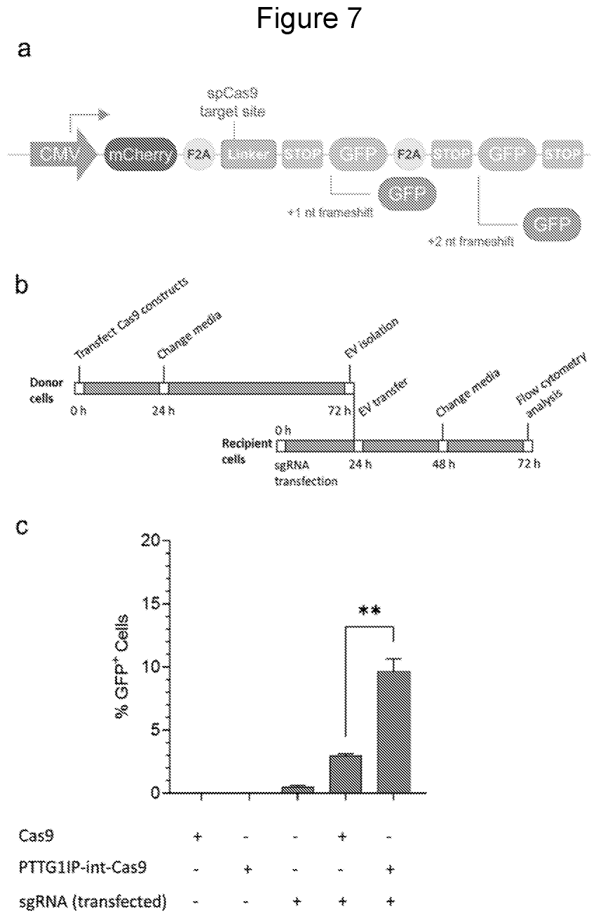

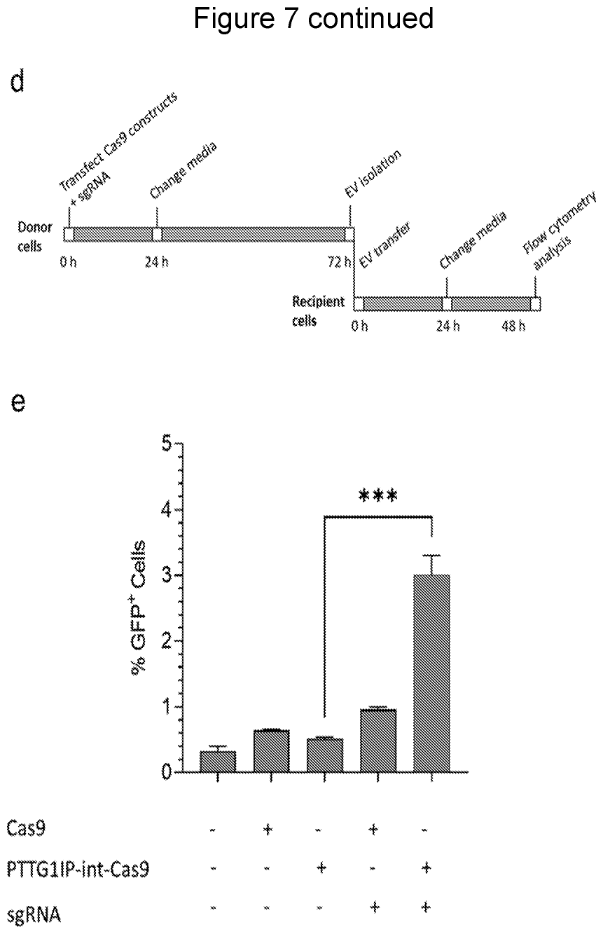

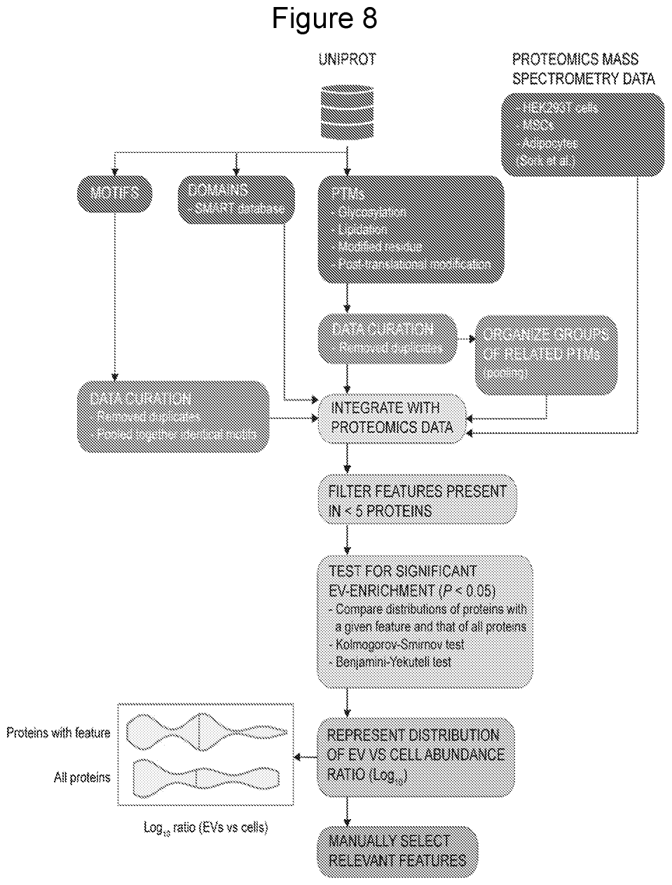

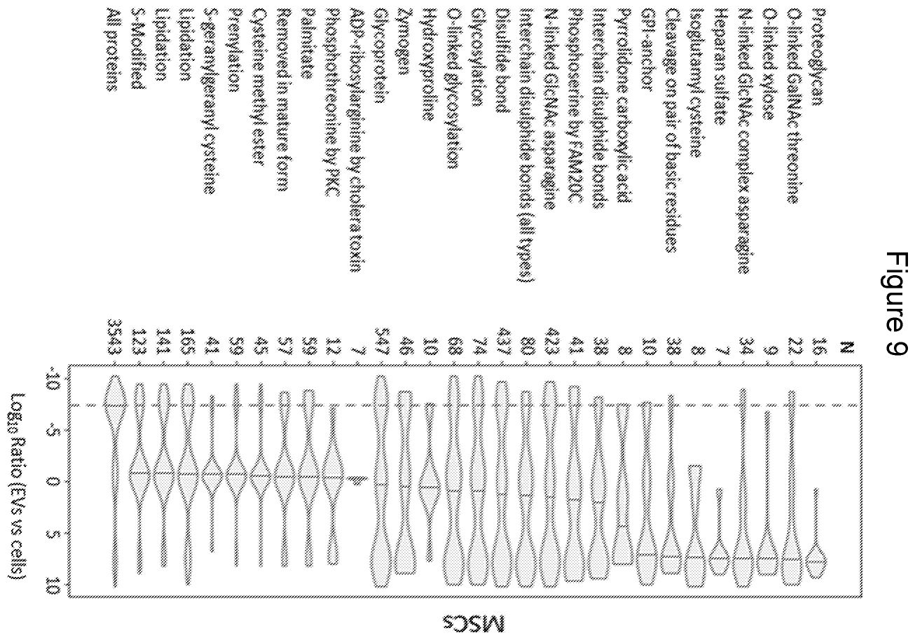

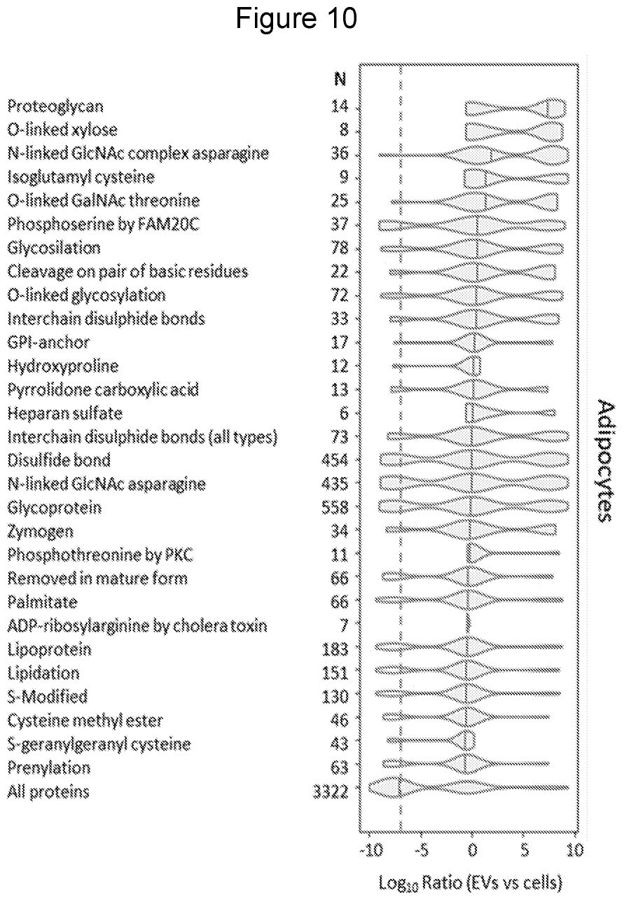

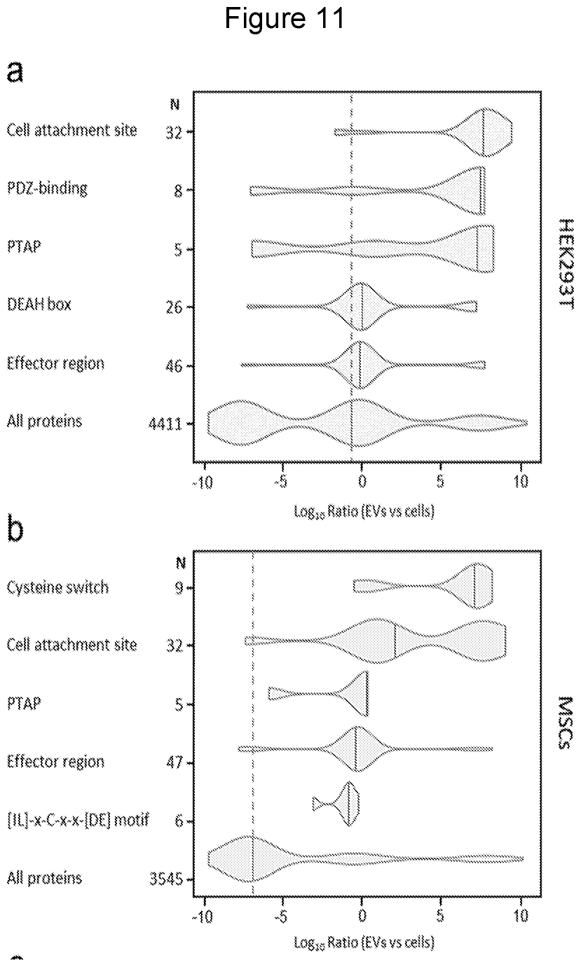

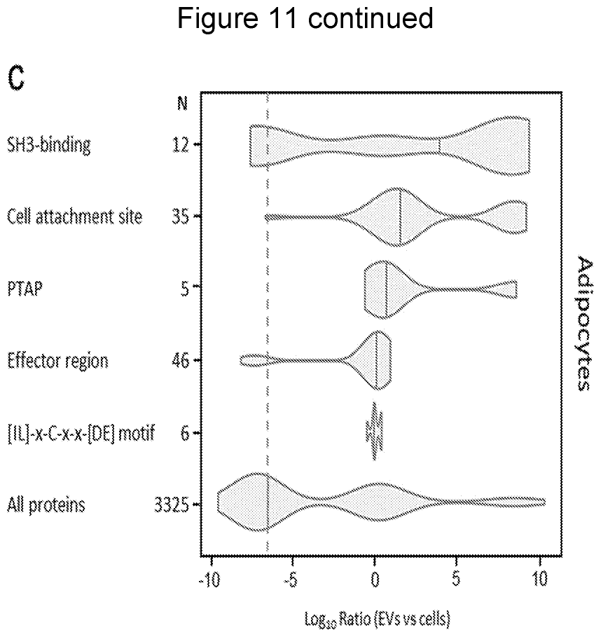

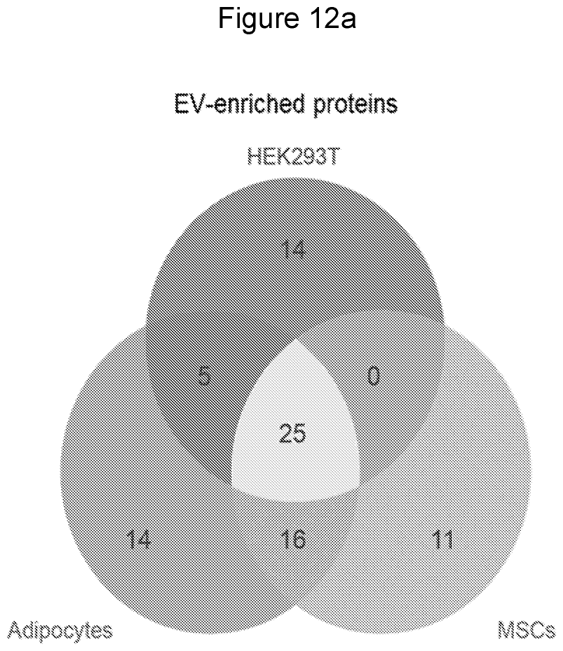

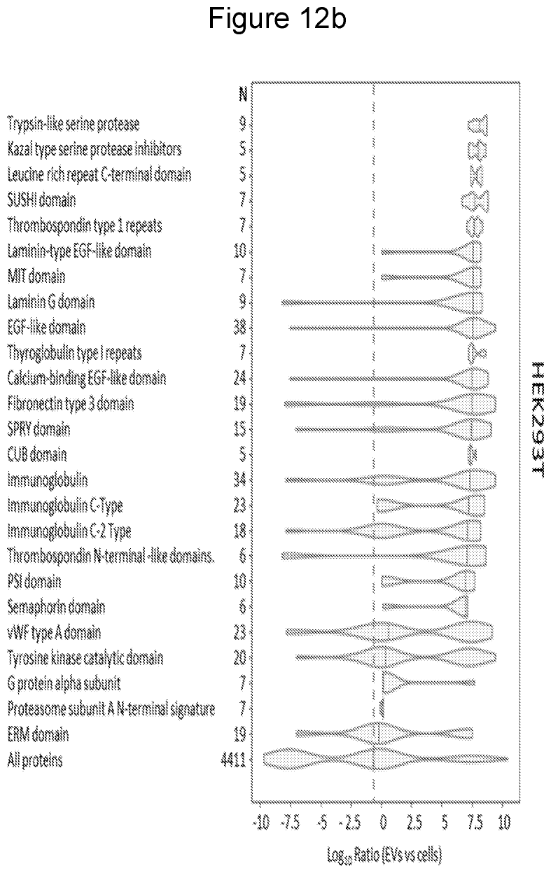

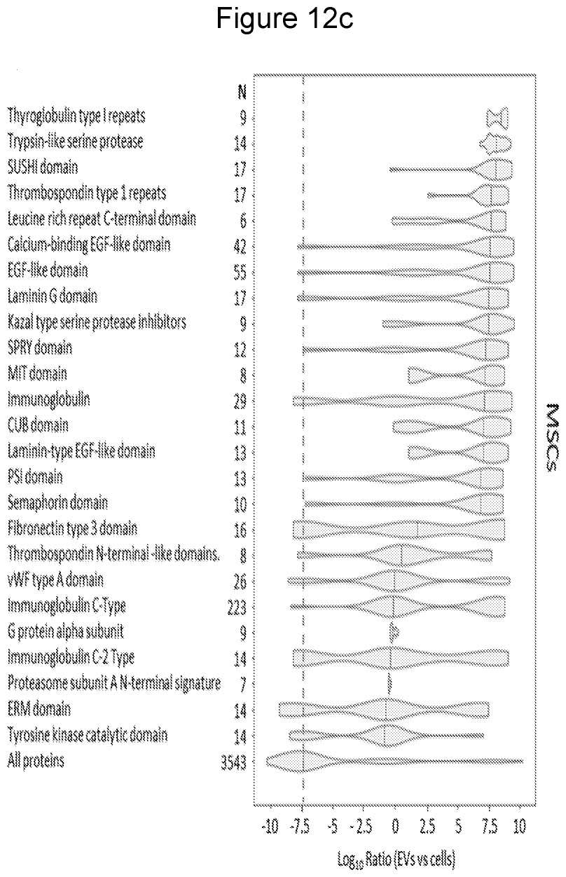

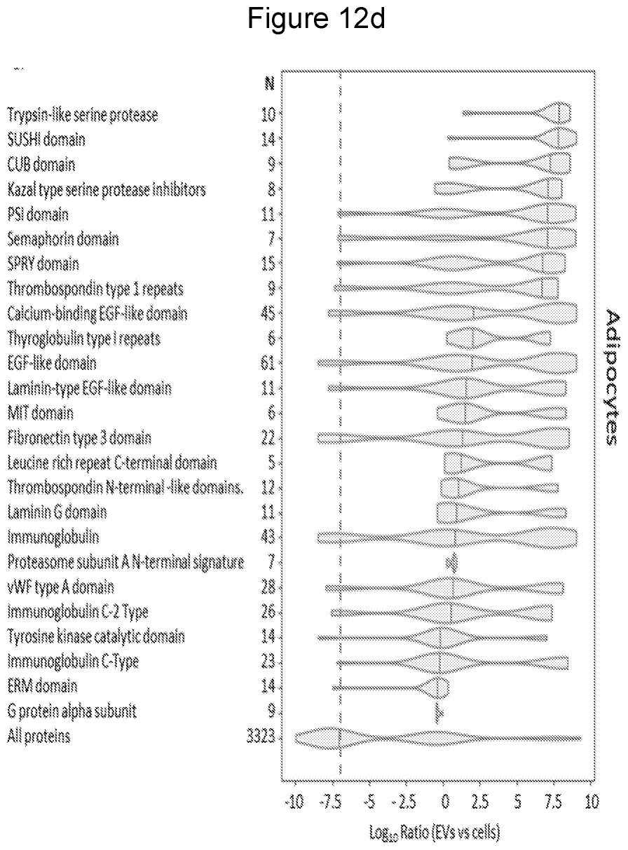

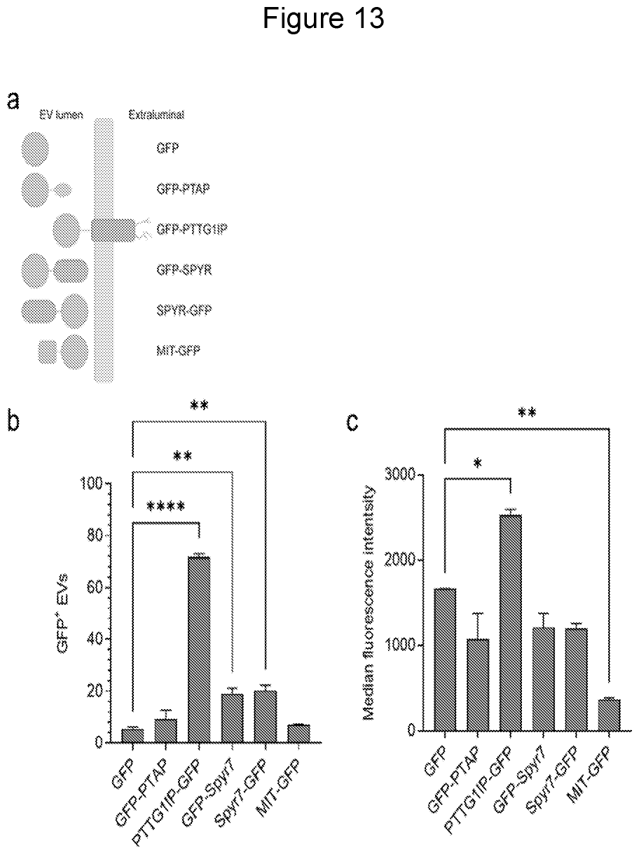

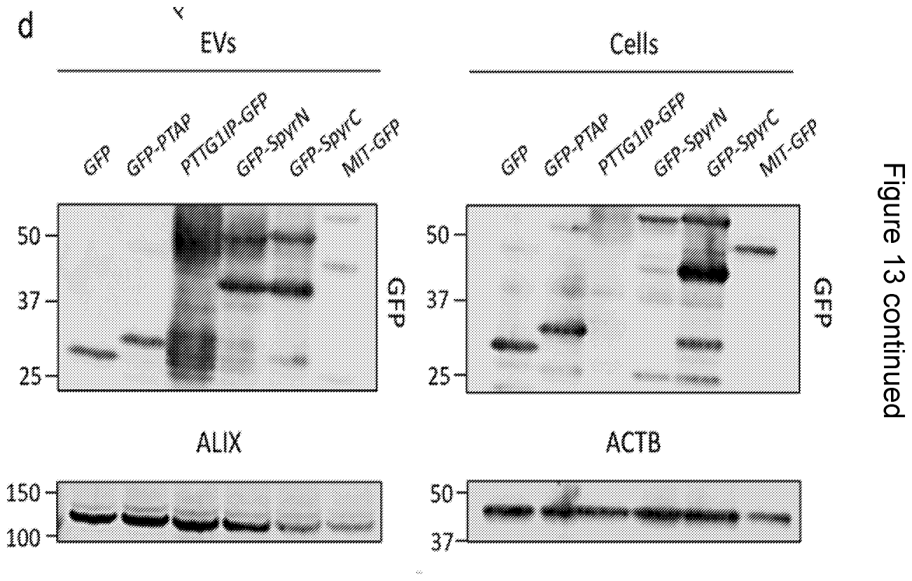

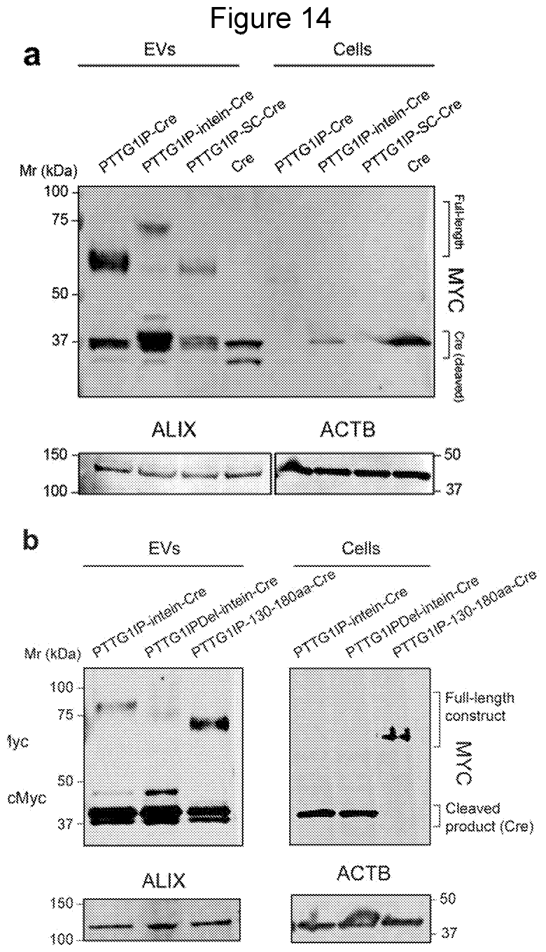

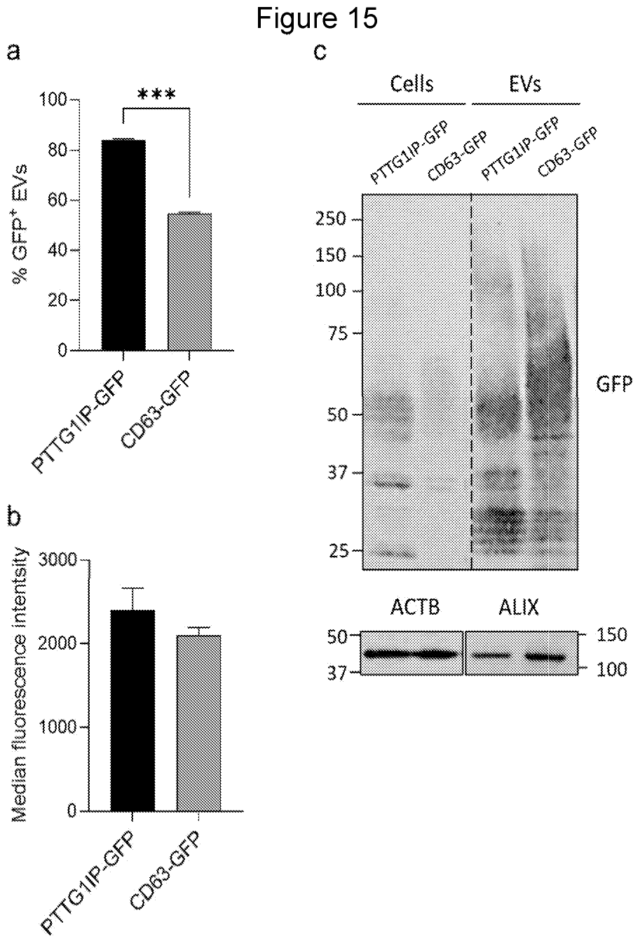

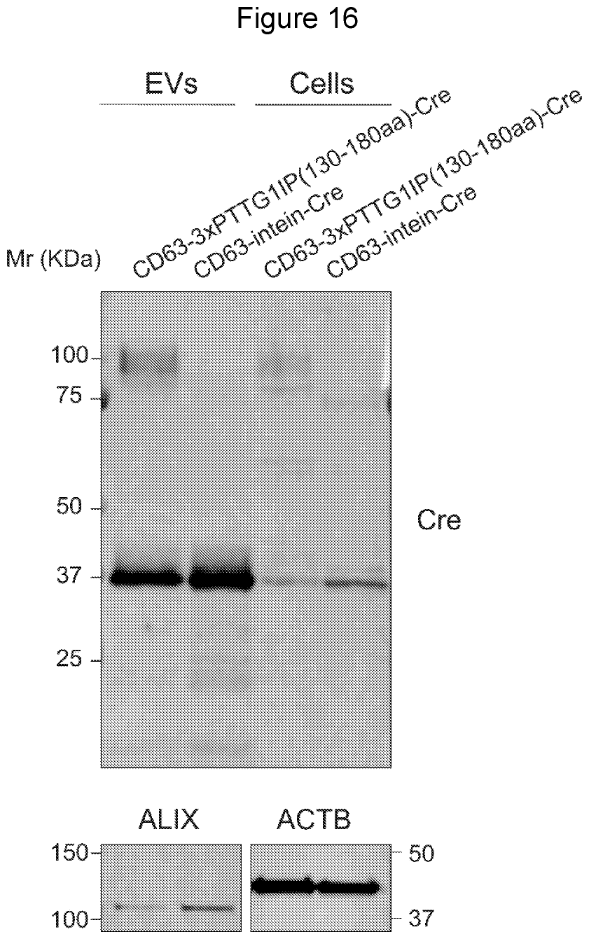

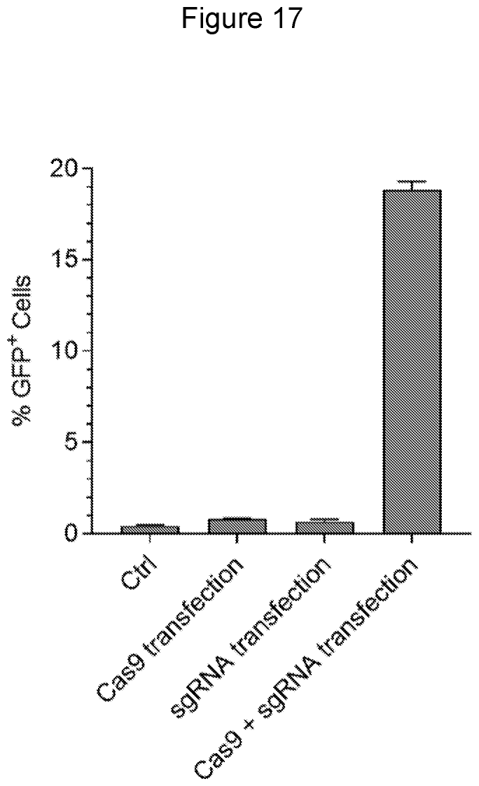

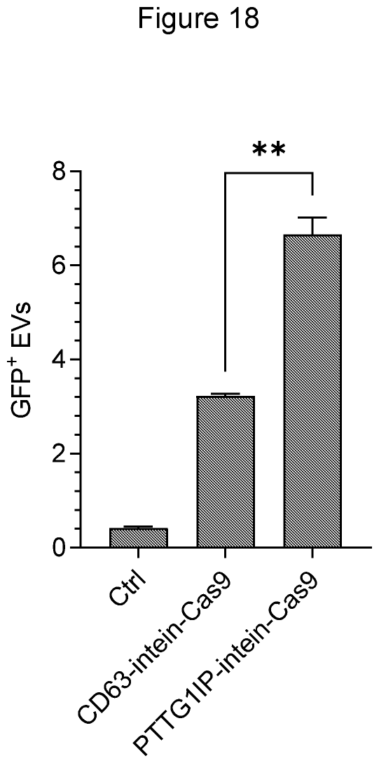

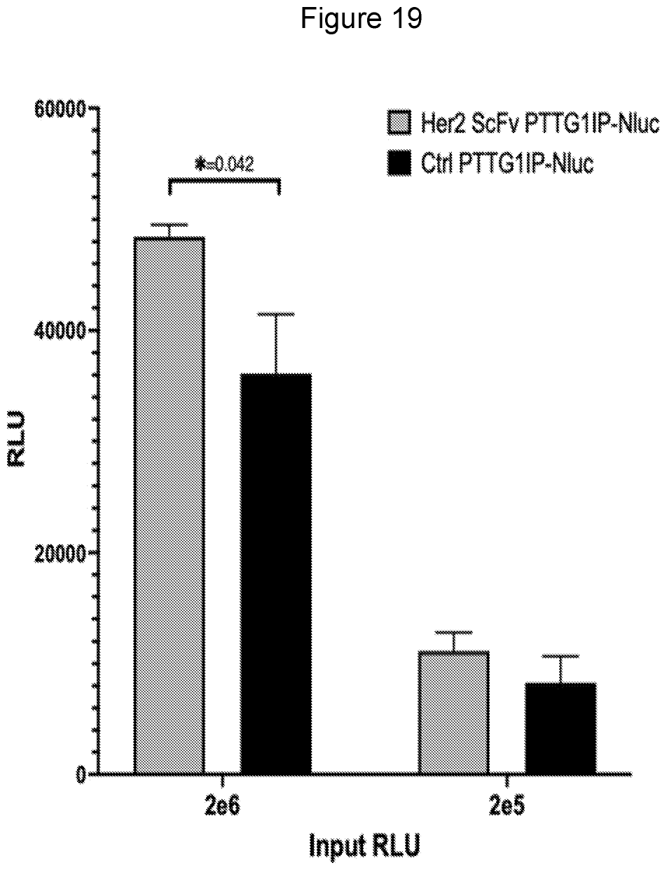

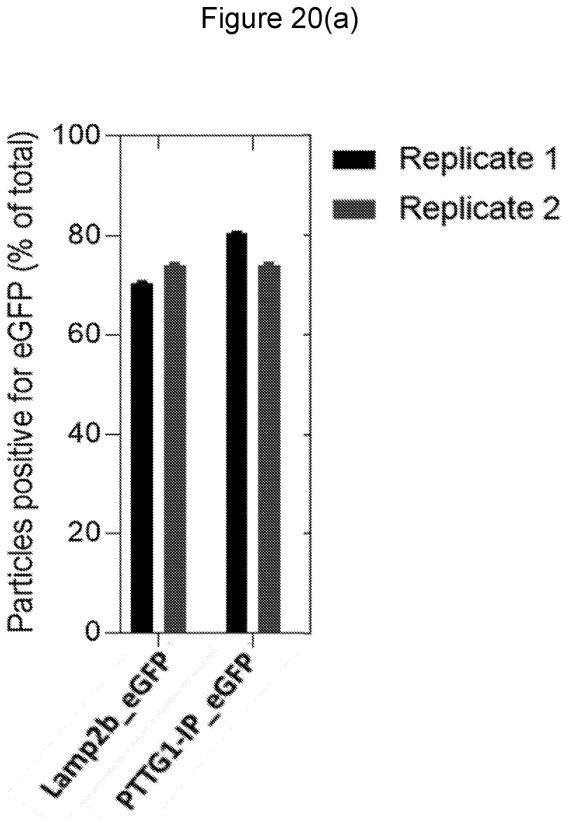

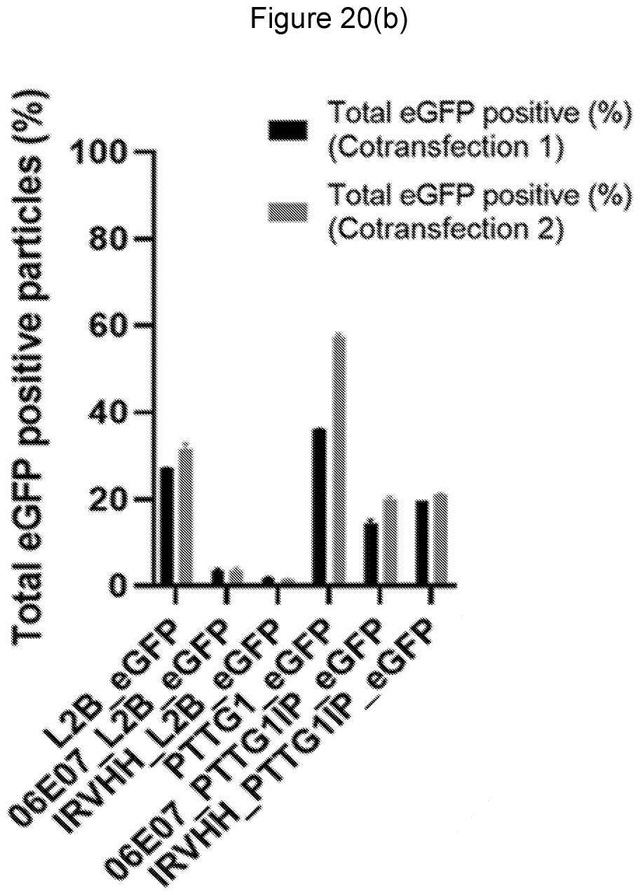

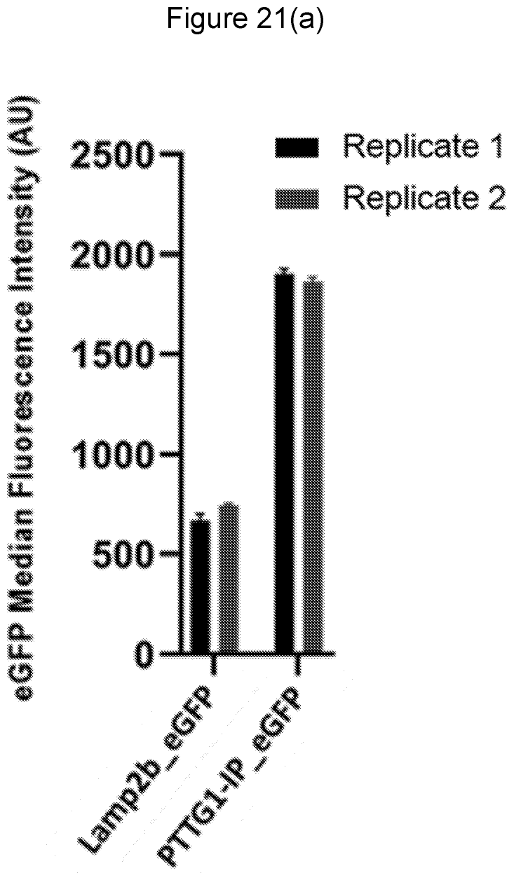

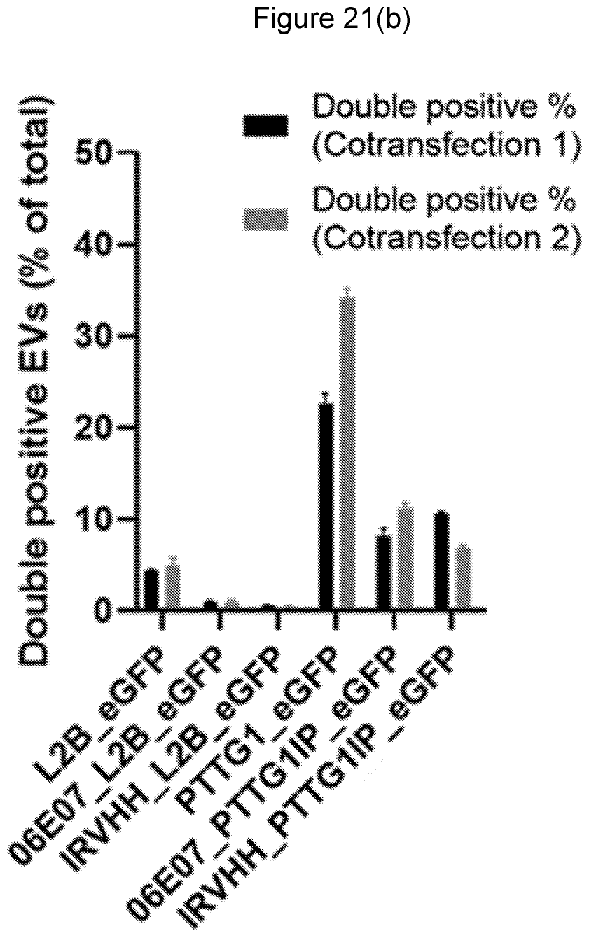

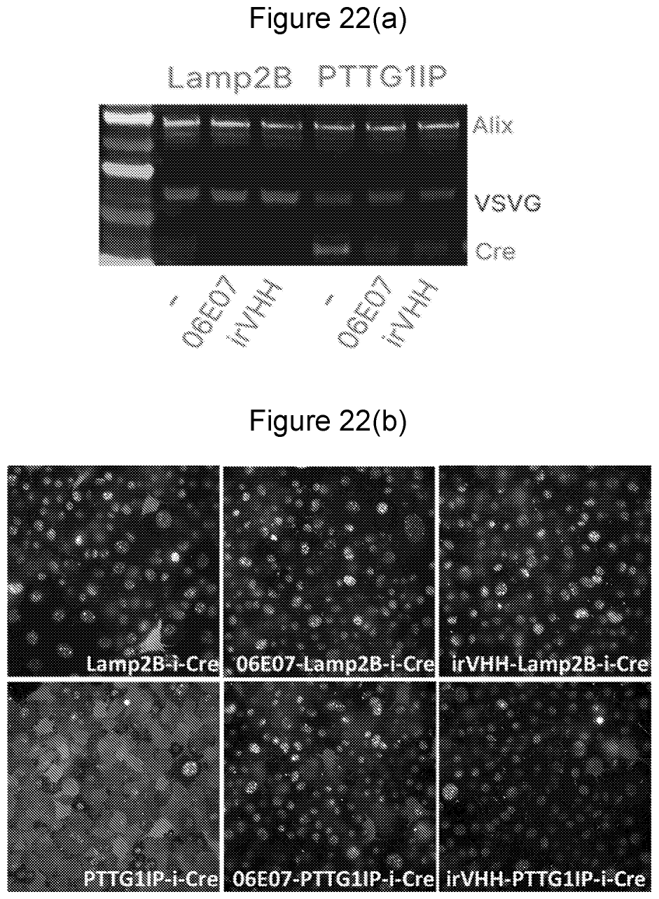

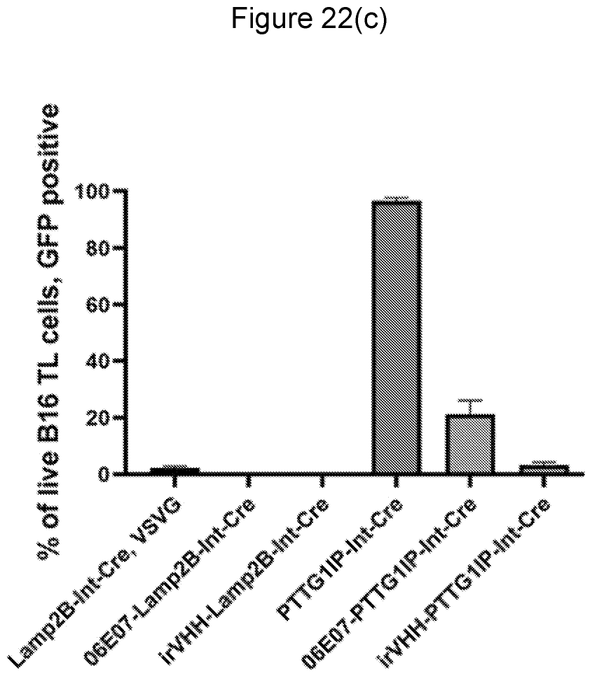

Custom Python scripts were used to identify EV-enriched protein features using mass spectrometry proteomics data and protein data from UniProt (FIG. 8). The first step of the analysis was the extraction of protein motif, domain, and post-translational modification (PTM) annotations from UniProt database. For PTMs and motifs, data were manually curated to remove duplicates and pool together identical features. Additionally, groups of related (e.g. glycosylation) PTMs were pooled and included as additional features. Data from protein features was then integrated with mass spectrometry proteomics data and features that appear on less than 5 proteins filtered out for reasons of statistical power. EV-enriched features were considered as those that were significant by both KW and KS tests, and which exhibited an increase in the EV to cell abundance ratio relative to the median of all proteins. EV-enriched features matching these criteria are shown in FIG. 1.

To identify protein EV-enriched PTMs, the bioinformatics analysis was performed in three different cell lines. 53, 39, and 75 EV-enriched PTMs were found on HEK293T, MSCs, and adipocytes, respectively (FIG. 1a). Among the identified PTMs, 25 were common across all 3 cell lines (FIG. 1a). Notably, N-linked GlcNAc asparagine (N-GlcNAc) was highly enriched and present in ˜10% of all proteins in the three datasets (FIG. 1b, FIGS. 9 and 10). Lipidation was also present on numerous proteins (˜5%), although to a lesser extent than glycosylation. Hydroxyproline, pyrrolidone carboxylic acid and kinase specific phosphorylation were also identified. The other six PTMs that were identified would be unfeasible to implement for protein EV-sorting due to their unspecific nature.

EV-enriched protein motifs and domains were also identified in the analysis and can be found on FIG. 11 and FIG. 12.

### Example 3—PTTG1IP Enables Highly Efficient EV-Loading in a N-Glycosylation Dependent Manner

Selected candidate features (PTAP motif, SPYR domain, MIT domain and N-linked glycosylation) were assessed for EV-loading by fusing them to GFP and analyzing their sorting to EVs (FIG. 13a) by Western Blot and single vesicle analysis. In the case of the MIT domain and N-linked glycosylation, it was decided to test the smallest protein containing the feature that could be found in our dataset to avoid altering protein conformations that may be important for domain or PTM functionality. MIT domain-containing protein 1 (MITD1) and PTTG1 Interacting Protein (PTTG1IP) were selected for MIT domain and N-glycosylation features, respectively. Importantly, PTTG1IP also has a PSI domain which was found to be EV-enriched in the bioinformatics analysis (FIG. 12). For the PTAP motif and SPRY domain these protein features were fused directly to GFP.

As shown in FIG. 13 b,d, GFP was highly enriched in the EVs when fused to PTTG1IP (˜75% of GFP⁺ vesicles) as compared to GFP alone (no scaffold) (˜3% of GFP⁺ vesicles). In addition, the mean fluorescence intensity of PTTG1IP-GFP vesicles was significantly higher (˜58%) that that of GFP, indicating a higher amount of GFP loading per vesicle (FIG. 13c). GFP fusion to SPRY domain also increased GFP EV-loading, although to a much lesser extent (˜23% of GFP⁺ vesicles).

N-glycosylation sites annotated in UniProt are mostly inferred according to sequence analysis. Thus, to confirm PTTG1IP N-glycosylation status, mutated residues were as follows: N45 (N45Q), N54 (N54Q) or N45 and N54 (2NQ) to glutamine to remove potential N-glycosylation sites and subjected HEK293T lysates from cells transfected with PTTG1IP mutants to PNGase F and Endonuclease H (EndoH) treatment (FIG. 2a). PNGase F removes all N-glycosylation sites and EndoH removes only those of high mannose or hybrid types. N-glycosylation removal can be observed by western blot as a shift in protein mobility (N-glycosylated proteins have a higher molecular weight). Treatment with PNGase F resulted in a shift of protein size from −55 kDa to −48 kDa, indicating that PTTG1IP is glycosylated (FIG. 2b). Single N-glycosylation mutants also showed a higher molecular weight than 2NQ mutant (FIG. 2b). Altogether, the shifts in molecular weight of the three mutants as compared to the WT suggest that both sites are N-glycosylated. To further explore the nature of the N-glycosylation sites, PTTG1IP mutants were treated with EndoH. As shown in FIG. 2b, a band shift is only observed for PTTG1IP N45Q, suggesting that N54 N-glycosylation is of high mannose or hybrid type while N45 is of complex type.

To determine if PTTG1IP EV-sorting was influenced by its N-glycosylation, EV-loading of PTTG1IP-GFP N-glycosylation mutants in HEK293T was assessed by Western Blot and single vesicle analysis. PTTG1IP 2NQ-GFP EV-loading was markedly reduced as compared to WT (˜25% of GFP+vesicles vs ˜80%) (FIG. 2 c,d,e). Mean fluorescence intensity of 2NQ mutant was also half of that of WT (FIG. 2e), which indicates that 2NQ GFP+vesicles have half the amount of GFP per vesicle. The N45Q and N54Q mutants also showed reduced loading (˜45% of GFP+vesicles) as compared to WT PTTG1IP. Importantly, the expression level of PTTG1IP mutants in cells were similar to that of WT PTTG1P, indicating that the lower sorting to EVs is not due to a lower expression by cells (FIG. 2c).

### Example 4—PTTG1IP can be Further Engineered to Improve its Applicability as an EV-Scaffold

PTTG1IP is a 180 amino acid single-pass type I transmembrane protein. It is composed by a signal peptide and a PSI domain harboring two N-glycosylation sites in its extracellular N-terminus, a transmembrane domain and an intracellular C-terminus consisting of an unstructured domain followed by a coiled coil region and two endocytosis signals (FIG. 3a). To improve the suitability of PTTG1IP as a scaffold for cargo loading to the EVs, two different PTTG1IP variants were engineered.

In the first variant, PTTG1IP-2YA, the two endocytosis signals of PTTG1IP (YXXL) at the C-terminus were mutated to YXXA (FIG. 3b). It has been previously shown that disrupting the endocytosis signal (YXXΦ motif, where Φ is a hydrophobic residue L, I, M, F, V) of CD63 re-directs the protein towards the microvesicle pathway rather than to the MVB exosome pathway¹⁵. p53 interaction with PTTG1IP intracellular domain has been reported to alter p53 activity¹⁶. In the second variant, the coiled coil region of PTTG1IP (130 to 164 amino acids) was deleted, as its intracellular domain has been reported to interact with p53¹⁶ (FIG. 2b). To determine the EV-loading capacity of the variants, HEK293T were transfected with WT PTTG1IP-GFP, PTTG1IP-2YA-GFP and PTTG1IP-130-164Del-GFP and their loading to EVs assessed by single vesicle analysis. Both variants result in a similar percentage of GFP+ particles (˜80%) (FIG. 3c). Nevertheless, the EV-mean fluorescence value for the PTTG1IP-2YA mutant was approximately twice the value of WT PTTG1IP, indicating that PTTG1IP-2YA was capable of loading double the amount of GFP per vesicle (FIG. 3d).

### Example 5—Assessment of Cre Recombinase Delivery to Reporter Cells

To assess the ability of PTTG1IP to functionally deliver protein cargo to recipient cells, delivery of Cre recombinase to reporter cells was assessed using a variety of constructs based on PTTG1IP, or CD63 as comparison (FIG. 4a). The latter is a well-described EV-enriched membrane protein of the tetraspanin family that has been previously used for EV-loading of therapeutic cargo^{6,8,9}. To achieve functional delivery in recipient cells, several steps need to be accomplished. Firstly, the EVs need to be loaded with sufficient protein cargo in the producer cells. When the protein cargo is loaded by fusing it to membrane proteins, such as PTTG1IP or CD63, this cargo needs to be released so that its able to reach its intracellular location in the target cell. Once taken up by the recipient cells (in most cases through the endocytic pathway), the EVs must escape endosomes before they are either re-exported to the extracellular space or degraded by the lysosomes. To overcome the cargo-release limitation, two different self-cleaving sequences from Myobacterium tuberculosis¹⁷ or Shewanella oneidensis⁸ were introduced (FIG. 4a). Ideally, these sequences should self-cleave at lower pH so that this only takes place once the cargo is in the EVs. To promote endosomal escape, vesicular stomatitis virus g (VSVG), a viral protein that mediates fusion of the EV and endosome membranes at lower pH therefore promoting endosomal escape¹⁹, was used. Importantly, VSVG and the cargo protein must be on the same EVs to achieve functional delivery. This will depend on the protein scaffold used for cargo loading and on whether VSVG and the scaffold protein are found on the same vesicle subpopulations.

Notably, Cre was released in the EVs from PTTG1IP-Cre fusion protein without the need of a self-cleaving protein as observed from the ˜38 kDa band that corresponds to the expected size of Cre (FIG. 14a). Nevertheless, when the intein is placed between PTTG1IP and Cre there was an increase of released Cre in the EVs (FIG. 14a). This was not the case for the other self-cleaving sequence from Shewanella oneidensis. To test delivery of Cre to recipient cells, HEK293T donor cells (host cells) were first transfected with several Cre constructs together with VSVG, and the resulting EVs transferred to Cre reporter cells (FIG. 4b). These reporter cells constitutively expressed RFP and contained a floxed stop signal followed by GFP. Upon Cre delivery, loxP sites recombine to excise the stop signal and GFP is expressed (FIG. 4c). T47D or HeLa reporter cells were treated with 5×10⁹ EVs/ml and activation was assessed 48h after by flow cytometry. PTTG1IP-intein-Cre showed the highest activation (˜98% in T47D and ˜85% in HeLa cells) while PTTG1IP-Cre induced activation of ˜40% in T47D and ˜10% in HeLa cells (FIG. 4d,e). Conversely, for Cre alone (no scaffold), activation was ˜3% in T47D and ˜1% in HeLa cells (FIG. 4d,e).

To take advantage of the natural (i.e. intrinsic) cleavage of PTTG1IP, a mutant was generated whereby the 130-180 sequence was duplicated at the C-terminus to further promote Cre release (FIG. 4a). It was hypothesized that the cleavage sequence was most likely at the end of PTTG1IP C-terminus given that no differences in molecular weight from WT Cre and Cre excised from PTTG1IP-Cre were observed (FIG. 14a).

To compare the efficiency of the various Cre constructs, an EV dose response of CD63-intein-Cre, PTTG1IP-intein-Cre, PTTG1IP-130-180aa-Cre and PTTG1IP130-164Del-intein-Cre in B16 and T47D reporter cells was performed. PTTG1IP-intein-Cre and PTTG1IP130-164Del-intein-Cre showed the higher efficiency followed by PTTG1IP-130-180aa-Cre (FIG. 4f,g). CD63-intein-Cre showed the lowest efficiency, especially in B16 cells (FIG. 4f,g). This may be due to the higher EV-loading capacity of PTTG1IP as compared to CD63 (FIG. 15) or a higher degree of co-localization between PTTG1IP and VSVG.

To determine if PTTG1IP 130-180 sequence could be used as an EV-cleavage sequence in a protein context distinct to that of PTTG1IP, cleavage of CD63-PTTG1IP130-180aa-Cre with CD63-intein-Cre were compared (FIG. 16). PTTG1IP130-180 exhibited increased cleavage resulting in Cre release in the EVs although to a lesser extent than the intein sequence. It was hypothesized that sequence 130-180 of PTTG1IP may contain a cleavage site that is cleaved predominantly in the EVs (due to the enzyme location or pH conditions). Therefore, if the minimal cleavage sequence was identified, several repeats instead of the intein sequence would be added. This would be important for therapeutic application given that the intein sequence is from Mycobacterium tuberculosis and is thus potentially immunogenic while PTTG1IP sequence is human. This may not be a concern when EVs are produced externally by producer cells given that the sequence will be inside the EVs, however if the EVs are to be produced endogenously in situ as previously shown^{20,21}, the expression of a bacterial peptide would most likely trigger an immune response towards the producer cells and its subsequent elimination.

### Example 6—Assessment of Cre Recombinase Delivery to Mice Tumour Xenografts

After having positive in vitro results, EV-mediated Cre delivery in vivo using mice harboring B16F10 Cre reporter cell tumours was assessed (FIG. 5a). EVs were isolated from HEK293T cells transfected with PTTG1IP-intein-Cre+VSVG, Cre+VSVG, or VSVG alone. After intra tumoural injection of 1×10¹⁰ EVs/mice, Cre activity was analysed by PCR (PCR using primers which span the reporter cassette and produce a 1,700 bp amplicon) and immunohistochemistry. Successful Cre recombination (indicated by a shorter 900 bp product (arrow)) was detected in tumours harvested from the three PTTG1IP-intein-Cre treated mice with recombination efficiencies ranging from 46-90%, while no recombination was detected with Cre treated mice or controls (VSVG alone EVs or PBS) (FIG. 5b). Similarly, GFP signal was only detected in tumours from mice injected with PTTG1IP-intein-Cre (FIG. 6). Together, these data suggest that PTTG1IP can be used as a scaffold for EV-mediated protein delivery in vivo.

### Example 7—Assessment of Cas9 and Cas9/sgRNA Complex Delivery to Reporter Cells

Having shown that PTTG1IP can be used for loading and delivering cargo protein via EVs, the ability of PTTG1IP in delivering more complex macromolecules, such as the CRISPR-Cas9 system was assessed. The CRISPR-Cas9 system represents a potent tool for gene editing and transcription regulation due to its high efficiency and specificity²². However, this potential is hampered by the lack of efficient delivery methods²³. Thus, the use of PTTG1IP to deliver Cas9 and Cas9-single-guide RNA (sgRNA) complexes to recipient cells was assessed. To assess Cas9 delivery, HEK293T stoplight reporter cells¹² were used (FIG. 17). These cells constitutively express RFP and express GFP upon Cas9 cleavage and indel formation (in 2 out of 3 of the events) (FIG. 7a). To test Cas9 delivery, HEK293T cells were transfected with Cas9 or PTTG1IP-Cas9 together with VSVG and EVs were isolated 72h later (FIG. 7b). 24h prior to EV transfer reporter cells were transfected with sgRNA and 48h later Cas9 activity was assessed by flow cytometry (FIG. 7b). ˜3% of reporter cells were GFP+after Cas9 EV-transfer while ˜10% of the cells were GFP+after PTTG1IP-intein-Cas9 transfer (FIG. 7c). Notably, Cas9 delivery was twice as efficient with PTTG1IP-intein-Cas9 than with CD63-intein-Cas9 (FIG. 18). To assess Cas9-sgRNA complex delivery, HEK293T cells were transfected with Cas9 or PTTG1IP-Cas9 together with VSVG and sgRNA (FIG. 7d). Efficiency in this case was 0.5% for Cas9 and 3% for PTTG1IP-intein-Cas9 (FIG. 7e). These results suggest that PTTG1IP is a suitable scaffold for delivery of more complex macromolecular cargoes.

### Example 8—Uptake of Targeted EVs Displaying HER2 scFv in PTTG1IP N Terminus by Human Breast Cancer Cell Line SKBR3

HEK-293T cells were seeded into 15 cm dishes, 10 million cells/dish 1 day before transfection. A total of 30 μg of plasmid (pLex CAG-PTTGIP-Nluc or pLex CAG-Her2 ScFV PTTGIP-NLuc) was mixed with 2 ml Opti-MEM in one tube for 5 min at room temperature and 45 μg of Polyethylenimine (PEI) (Sigma) was mixed with 2 ml Opti-MEM in another tube with the same incubation conditions. Then, the plasmid and PEI were mixed in one tube and incubated for 20 min at room temperature. The DNA-PEI mixture was added into the medium in a dropwise manner. The medium was changed to Opti-MEM with 1% Anti-anti 3-4 hours post-transfection, and EVs were harvested 48 hours after the medium change.

Conditioned media (CM) was pre-cleared first by a low-speed centrifugation step (500×g for 10 min) followed by centrifugation at 2,000×g for 10-20 min to remove larger particles and debris. Samples were subsequently filtered through a syringe filter (VWR) with a cellulose acetate membrane with a 0.22 μm pore size to remove any remaining larger particles. The CM was then ultra-filtrated using Amicon Ultra-15 100 kDa (Millipore) spin filter at 4000×g for 30 min to concentrate the CM. EVs were then isolated with UF/size exclusion chromatography (SEC) as described above. Particle concentration and size were analyzed with NTA in scatter mode. A range of EV doses based on total luminescence were added to human breast cancer cell line SKBR3 cells seeded the day before at a density of 1×10⁴ cells per well in a 96-well plate. Cells were incubated for 2 hours at 37° C., 5% C02 atmosphere. After incubation, the cells were washed twice with PBS and lysed in 100 μl of Dulbecco's PBS (Invitrogen) and 0.1% TritonX-100. The plate was then incubated on an orbital shaker at room temperature for 10 min for complete lysis of the cells. The cell lysate was then analyzed for luciferase activity using the appropriate substrates as detailed above.

To determine if PTTG1IP scaffold could be used for displaying targeting proteins on the EV surface, the uptake of Nluc-loaded EVs displaying HER2 fused to PTTG1IP by human breast cancer cell line SKBR3 was assessed. EVs derived from cells expressing HER2 scFv-PTTG1IP-Nluc or PTTG1IP-Nluc were incubated with recipient cells, and luciferase activity in cells was analyzed after 2h. Luciferase activity of recipient cells treated with HER2 scFv-PTTG1IP-Nluc EVs was higher than that of cells treated with control EVs without HER2 scFv (PTTG1IP-Nluc)—FIG. 19. This suggests that PTTG1IP scaffold is able to display targeting moieties on the EV surface which result in enhanced uptake by recipient cells.

### Example 9—Assessment of EV Loading of PTTG1IP, as Compared to Single-Pass EV Scaffold Protein Lamp2B

PTTG1IP is a single-pass transmembrane protein. The use of single pass transmembrane proteins in EV engineering is associated with a number of advantages. In particular, single pass transmembrane proteins allow for increased ease of engineering moieties that are to be displayed on the EV surface. However, there are problems with commonly used single-pass transmembrane proteins, including low representation of these protein on the EV surface and poor EV co-localisation with other proteins (such as VSVG).

The ability of PTTG1IP to mediate cargo loading, as compared to another single-pass EV scaffold protein, Lamp2B, was assessed.

HEK VPC 2.0 cells were transfected with the following constructs:
Lamp2B-eGFP (eGFP fused to the C-terminus of Lamp2B)
PTTG1IP-eGFP (eGFP fused to the C-terminus of PTTG1IP)

72 hrs after the HEK VPC 2.0 cells were transfected, the EVs were harvest and isolated by performing a first centrifugation at 300×G for 5 mins and discarding the cells. Performing a second centrifugation at 700×G for 10 mins and collecting the supernatant. Performing a third centrifugation at 4000×G for 20 mins and collecting the supernatant. Passing the supernatant through a 0.2 μm filtration, then using a spin filter (100 kDa MWCO) to concentrate the sample to <20 ml. An ultracentrifugation step (100,000×g, 2 hr) was then performed and the pellet was then resuspended in 300 μl 0.2% HSA in PBS.

Florescence was then detected in the individual EVs from the purified samples using a NanoFCM Flow Nanoanalyzer. The proportion of GFP positive EVs was comparable in the EV sample purified from HEK cells that were transfected with the Lamp2B-eGFP construct and the EV sample purified from HEK cells that were transfected with PTTG1IP-eGFP construct (FIG. 20(a)). However, the median fluorescence intensity of the EVs was higher in the EV sample purified from HEK cells that were transfected with the with PTTG1IP-eGFP construct, as compared with the EV sample purified from HEK cells that were transfected with Lamp2B-eGFP construct (FIG. 20(b)). This shows that engineered PTTG1IP is better represented on the surface of engineered EVs and that more cargos per loaded EV are obtained, as compared to Lamp2B. Therefore, surprisingly, using PTTG1IP as a scaffold for loading cargo would be expected to result in EVs that are able to deliver more cargo to target cells.

### Example 10 Assessment of EV Co-Localisation of VSVG and Engineered PTTG1IP, as Compared to Co-Localisation of VSVG and Engineered Protein Lamp2B

The ability of engineered PTTG1IP to be co-expressed in an EV alongside VSVG, as compared to Lamp2B, was investigated.

HEK VPC 2.0 cells were transfected with the following constructs:
Lamp2B-eGFP (eGFP fused to the C-terminus of Lamp2B)
06E07-Lamp2b-eGFP (eGFP fused to the C-terminus of Lamp2B and a nanobody that binds to human transferrin receptor fused to the N-terminus of Lamp2B)
IRVHH-Lamp2b-eGFP (eGFP fused to the C-terminus of Lamp2B and a nanobody that binds to human respiratory syncytial virus fusion protein fused to the N-terminus of Lamp2B)
PTTG1IP-eGFP (eGFP fused to the C-terminus of PTTG1IP)
06E07-PTTG1IP-eGFP (eGFP fused to the C-terminus of PTTG1IP and a nanobody that binds to human transferrin receptor fused to the N-terminus of PTTG1IP)
IRVHH-PTTG1IP-eGFP (eGFP fused to the C-terminus of PTTG1IP and a nanobody that binds to human respiratory syncytial virus fusion protein fused to the N-terminus of PTTG1IP).

72 hrs after the HEK VPC 2.0 cells were transfected, the EVs were harvest and isolated as described in Example 9.

Florescence was then detected in the individual EVs from the purified samples using a NanoFCM Flow Nanoanalyzer. The proportion of GFP positive EVs was increased in the EV sample purified from HEK cells that were transfected with the VSVG construct and the PTTG1IP-eGFP construct, as compared with the sample purified from HEK cells that were transfected with the VSVG construct and the Lamp2B-eGFP construct (FIG. 21).

The proportion of GFP positive EVs is dramatically decreased when the HEK producer cells are transfected with an N-terminally engineered Lamp2B construct, alongside VSVG. Whilst a decrease GFP positive EVs was still observed when PTTG1IP is N-terminally engineered, expression of N-terminally engineered PTTG1IP constructs allows for an increased proportion of GFP positive EVs to be produced as compared to N-terminally engineered Lamp2B constructs. In this respect, see FIG. 21.

The above demonstrates that engineered PTTG1IP is expressed on a higher proportion of EVs than engineered Lamp2B when the producer cells have been co-transfected with the engineered scaffold proteins alongside a second construct (e.g. VSVG). This is especially the case where the scaffolds are N-terminally engineered. Therefore, surprisingly, using PTTG1IP as a scaffold not only produces a higher yield of engineered EVs per batch but also allows for more efficient production of cargo loaded EVs with greater flexibility for engineering additional constructs into the EV product without loss of expression.

The proportion of EVs that are positive for both VSVG and GFP was also detected using the NanoFCM Flow Nanoanalyzer. VSVG was detected through use of a mouse anti-VSVG antibody (Absolute Antibody) that had been labelled in-house using an Invitrogen Alexa Fluor antibody labelling kit. The proportion of VSVG and GFP positive EVs was increased in the EV sample purified from HEK cells that were transfected with the VSVG construct and the PTTG1IP-eGFP construct, as compared with the sample purified from HEK cells that were transfected with the VSVG construct and the Lamp2B-eGFP construct (FIG. 21(b), compare columns 1 and 2 with columns 7 and 8).

As with the proportion of GFP positive EVs, the proportion of VSVG and GFP positive EVs is dramatically decreased when the HEK producer cells are transfected with an N-terminally engineered Lamp2B construct, alongside VSVG (FIG. 21(b), compare columns 1 and 2 with columns 3 to 6). Whilst a decrease VSVG and GFP positive EVs is also observed when PTTG1IP is N-terminally engineered (FIG. 21(b), compare columns 7 and 8 with columns 9 to 12), expression of N-terminally engineered PTTG1IP constructs allows for an increased proportion of VSVG and GFP positive EVs to be produced as compared to N-terminally engineered Lamp2B constructs (FIG. 21(b), compare columns 9 to 12 with columns 3 to 6).

The above demonstrates that VSVG and engineered PTTG1IP are co-expressed on a higher proportion of EVs, as compared with VSVG and engineered Lamp2B. This is especially the case where the scaffolds are N-terminally engineered. Therefore, surprisingly, using PTTG1IP as a scaffold allows for more efficient production of EVs that are loaded with multiple exogenous polypeptides, which is greatly expands the possibilities for cargo loading and thus multiplexing of cargos.

### Example 11 Assessment of Functional Delivery of PTTG1IP and Lamp2B for Functional Delivery of Cre Recombinase In Vitro

The ability of engineered PTTG1IP to functionally deliver Cre recombinase to cells, as compared to Lamp2B, was also investigated.

HEK VPC 2.0 cells were transfected with the following constructs:
Lamp2B-intein-Cre (Cre fused to the C-terminus of Lamp2B via a self-cleaving intein construct)
06E07-Lamp2b-intein-Cre (Cre fused to the C-terminus of Lamp2B via a self-cleaving intein construct and a nanobody that binds to human transferrin receptor fused to the N-terminus of Lamp2B)
IRVHH-Lamp2b-eGFP intein-Cre (Cre fused to the C-terminus of Lamp2B via a self-cleaving intein construct and a nanobody that binds to human respiratory syncytial virus fusion protein fused to the N-terminus of Lamp2B)
PTTG1IP-intein-Cre (Cre fused to the C-terminus of PTTG1IP via a self-cleaving intein construct)
06E07-PTTG1IP-intein-Cre (Cre fused to the C-terminus of PTTG1IP via a self-cleaving intein construct and a nanobody that binds to human transferrin receptor fused to the N-terminus of PTTG1IP)
IRVHH-PTTG1IP-intein-Cre (Cre fused to the C-terminus of PTTG1IP via a self-cleaving intein construct and a nanobody that binds to human respiratory syncytial virus fusion protein fused to the N-terminus of PTTG1IP).

72 hrs after the HEK VPC 2.0 cells were transfected, the EVs were harvest and isolated as described in Example 9.

The level of Cre Recombinase, VSVG and Alix (loading control) expression in the different EV samples was detected by western blot. Cre expression was increased in the EV sample purified from HEK cells that were transfected with the VSVG construct and the PTTG1IP-intein-Cre construct, as compared with the sample purified from HEK cells that were transfected with the VSVG construct and the Lamp2B-intein-Cre construct (FIG. 22(a), compare samples 1 and 4).

Cre expression was no longer detected in the EV sample from the HEK cells transfected with the N-terminally engineered Lamp2B constructs, alongside VSVG (FIG. 22(a) samples 2 and 3). Whilst a decrease Cre expression is also observed in the EV samples from the HEK cells transfected with the N-terminally engineered PTTG1IP constructs and VSVG (FIG. 22(a) compare samples 5 and 6 to sample 4), Cre is still detected in these samples, thus the N-terminally engineered PTTG1IP constructs allows for an increased Cre expression in EVs as compared to the N-terminally engineered Lamp2B constructs (FIG. 22(a) compare samples 2 and 3 with samples 5 and 6). VSVG expression is slightly reduced in the EV samples from cells transfected with VSVG alongside the engineered PTTG1IP constructs, as compared to cells transfected with VSVG alongside the engineered Lamp2B constructs.

The above demonstrates that cargoes fused to PTTG1IP are expressed at a higher level in EVs than cargos fused to Lamp2B, when the producer cells have been co-transfected with the engineered scaffold proteins alongside a second construct (e.g. VSVG). This is especially the case where the scaffolds are N-terminally engineered. Therefore, surprisingly, using PTTG1IP as a scaffold allows for more improved EV loading. The reduction in VSVG expression also indicates that using PTTG1IP as a scaffold may allow for the production of EVs with reduced immunogenicity.

The EV samples were added to B16 Traffic Light cells, which express a constitutively expressed construct comprising RFP followed by a STOP codon, which is flanked by LoxP sites, and is upstream of a GFP sequence. In the absence of Cre recombinase, RFP is expressed and GFP is not. However, when Cre recombinase is present in these cells, the RFP and STOP codon are excised and thus GFP starts to be expressed and RFP is no longer expressed.

40 hours after the EVs were added to the B16 Traffic Light cells, the cells were imaged (FIG. 22(b)), and the percentage of GFP positive cells were quantified (FIG. 22(c)). The proportion of GFP positive cells was increased in cells treated with the EV sample purified from HEK cells that were transfected with the VSVG construct and the PTTG1IP-intein-Cre construct, as compared with the sample purified from HEK cells that were transfected with the VSVG construct and the Lamp2B-intein-Cre construct (FIG. 22(c), compare columns 1 and 4).

No GFP positive cells were detected in the cells treated with the EV sample from the HEK cells transfected with the N-terminally engineered Lamp2B constructs, alongside VSVG (FIG. 22(c), columns 2 and 3). Whilst a decrease in the proportion of GFP positive cells was also observed in the cells treated with EV samples from the HEK cells transfected with the N-terminally engineered PTTG1IP constructs and VSVG (FIG. 22(c), compare column 5 and 6 to column 4), some GFP positive cells were detected, thus the N-terminally engineered PTTG1IP constructs allows for the production of EVs that mediate increased functional delivery of Cre, as compared to the N-terminally engineered Lamp2B constructs (FIG. 22(c), compare column 2 and 3 with column 5 and 6).

The above demonstrates that higher levels of functional delivery of cargo are achieved when the cargo is fused to PTTG1IP, as compared to when the cargo is fused to Lamp2B. This is especially the case where the scaffolds are also N-terminally engineered. Therefore, surprisingly, using PTTG1IP as a scaffold allows for the production of engineered EVs with higher levels of bioactivity and greater flexibility of cargo loading.

Sequence Information
Sequence of the PTTG1IP peptide
SEQ ID NO: 1
MAPGVARGPTPYWRLRLGGAALLLLLIPVAAAQEPPGAACSQNTNKTCEE
CLKNVSCLWCNTNKACLDYPVTSVLPPASLCKLSSARWGVCWVNFEALII
TMSVVGGTLLLGIAICCCCCCRRKRSRKPDRSEEKAMREREERRIRQEER
RAEMKTRHDEIRKKYGLFKEENPYARFENN
Sequence of PTTG1IP “2YA” peptide
SEQ ID NO: 2
MAPGVARGPTPYWRLRLGGAALLLLLIPVAAAQEPPGAACSQNTNKTCEE
CLKNVSCLWCNTNKACLDYPVTSVLPPASLCKLSSARWGVCWVNFEALII
TMSVVGGTLLLGIAICCCCCCRRKRSRKPDRSEEKAMREREERRIRQEER
RAEMKTRHDEIRKKAGLFKEENPAARFENN
Sequence of the PTTG1IP “130-180
amino acid” peptide
SEQ ID NO: 3
MAPGVARGPTPYWRLRLGGAALLLLLIPVAAAQEPPGAACSQNTNKTCEE
CLKNVSCLWCNTNKACLDYPVTSVLPPASLCKLSSARWGVCWVNFEALII
TMSVVGGTLLLGIAICCCCCCRRKRSRKPYGLFKEENPYARFENN
Sequence of the PTTG1IP “130-164del”
peptide
SEQ ID NO: 4
MAPGVARGPTPYWRLRLGGAALLLLLIPVAAAQEPPGAACSQNTNKTCEE
CLKNVSCLWCNTNKACLDYPVTSVLPPASLCKLSSARWGVCWVNFEALII
TMSVVGGTLLLGIAICCCCCCRRKRSRKPDRSEEKAMREREERRIRQEER
RAEMKTRHDEIRKKYGLFKEENPYARFENNDPPVATDRSEEKAMREREER
RIRQEERRAEMKTRHDEIRKKYGLFKEENPYARFENN
Sequence of the PTTGIIP “self-cleaving”
peptide
SEQ ID NO: 5
DRSEEKAMREREERRIRQEERRAEMKTRHDEIRKKYGLFKEENPYARFEN
N
Sequence of the DPPVAT protein linker
SEQ ID NO: 6
DPPVAT

1. Mathieu, M., Martin-Jaular, L., Lavieu, G. & Théry, C. Specificities of secretion and uptake of exosomes and other extracellular vesicles for cell-to-cell communication. Nat. Cell Biol. 21, 9-17 (2019).
2. Van Niel, G., D'Angelo, G. & Raposo, G. Shedding light on the cell biology of extracellular vesicles. Nat. Rev. Mol. Cell Biol. 19, 213-228 (2018).
3. Loughlin, A. J. O., Woffindale, C. A. & Wood, M. J. A. Exosomes and the Emerging Field of Exosome-Based Gene Therapy. Curr. Gene Ther. 262-274 (2012).
4. Villarroya-Beltri, C., Baixauli, F., Gutidrrez-Vizquez, C., Sinchez-Madrid, F. & Mittelbrunn, M. Sorting it out: Regulation of exosome loading. Semin. Cancer Biol. 28, 3-13 (2014).
5. Ageta, H. & Tsuchida, K. Post-translational modification and protein sorting to small extracellular vesicles including exosomes by ubiquitin and UBLs. Cell. Mol. Life Sci. (2019) doi:10.1007/s00018-019-03246-7.
6. Hung, M. E. & Leonard, J. N. A platform for actively loading cargo RNA to elucidate limiting steps in EV-mediated delivery. J. Extracell. Vesicles 5, (2016).
7. Liang, G. et al. Engineered exosome-mediated delivery of functionally active miR-26a and its enhanced suppression effect in HepG2 cells. Int. J. Nanomedicine Volume 13, 585-599 (2018).
8. Kanuma, T. et al. CD63-Mediated Antigen Delivery into Extracellular Vesicles via DNA Vaccination Results in Robust CD8+T Cell Responses. J. Immunol. 198, 4707-4715 (2017).
9. Yao, X. et al. Engineered extracellular vesicles as versatile ribonucleoprotein delivery vehicles for efficient and safe CRISPR genome editing. J. Extracell. Vesicles (2021) doi:10.1002/jev2.12076. 10. Martin Perez, C., Conceigao, M., Raz, R., Wood, M. J. A. & Roberts, T. C. Enhancing the Therapeutic Potential of Extracellular Vesicles Using Peptide Technology. Methods Mol. Biol. 2383, 119-141 (2022).
11. Ran, F. A. et al. Genome engineering using the CRISPR-Cas9 system. Nat. Protoc. 8, 2281-2308 (2013).
12. Jong, O. G. De et al. A CRISPR-Cas9-based reporter system for single-cell detection of extracellular vesicle-mediated functional transfer of RNA Olivier. Nat. Commun. 1-13 doi:10.1038/s41467-020-14977-8.
13. Nakamura, K., Kosaka, N., Nakatani, M., Uezumi, A. & Ide, T. UBL3 modification influences protein sorting to small extracellular vesicles. Nat. Commun. 1-3 (2018) doi:10.1038/s41467-018-06197-y.
14. VerPlank, L. et al. Tsg101, a homologue of ubiquitin-conjugating (E2) enzymes, binds the L domain in HIV type 1 Pr55(Gag). Proc. Natl. Acad. Sci. U.S.A 98, 7724-7729 (2001).
15. Mathieu, M. et al. Specificities of exosome versus small ectosome secretion revealed by live intracellular tracking of CD63 and CD9. Nat. Commun. 12, 1-18 (2021).
16. Read, M. L. et al. The PTTG1-binding factor (PBF/PTTG1IP) regulates p53 activity in thyroid cells. Endocrinology 155, 1222-1234 (2014).
17. Wood, D. W., Wu, W., Belfort, G., Derbyshire, V. & Belfort, M. A genetic system yields self-cleaving inteins for bioseparations. Nat. Biotechnol. 889-892 (1994).
18. Osipiuk, J., Mulligan, R., Bargassa, M., Hamilton, J. E. & Cunningham, M. A. Characterization of Member of DUF1888 Protein Family, J. Biol. Chem. 287, 19452-19461 (2012).
19. Meyer, C. et al. Pseudotyping exosomes for enhanced protein delivery in mammalian cells. Int. J. Nanomedicine 12, 3153-3170 (2017).
20. Kojima, R. et al. Designer exosomes produced by implanted cells intracerebrally deliver therapeutic cargo for Parkinson's disease treatment. Nat. Commun. 9, 1-10 (2018).
21. Duarte-Sanmiguel, S. et al. In Situ Deployment of Engineered Extracellular Vesicles into the Tumor Niche via Myeloid-Derived Suppressor Cells. Adv. Healthc. Mater. 11, (2022).
22. Doudna, J. A. & Charpentier, E. The new frontier of genome engineering with CRISPR-Cas9. Science (80-.). 346, 1258096-1258096 (2014).
23. Modell, A. E., Lim, D., Nguyen, T. M., Sreekanth, V. & Choudhary, A. CRISPR-based therapeutics: current challenges and future applications. Trends Pharmacol. Sci. 43, 151-161 (2022).
24. Sork, H. et al. Heterogeneity and interplay of the extracellular vesicle small RNA transcriptome and proteome. Sci. Rep. 8, (2018).
25. Henikoff and Henikoff Proc. Natl. Acad. Sci. USA 89: 10915-10919 (1992).
26. Karlin and Altschul Proc. Natl. Acad. Sci. USA 90: 5873-5787 (1993).
27. Devereux et al Nucleic Acids Research 12, 387-395 (1984).

### ASPECTS OF THE INVENTION

1. A composition comprising an extracellular vesicle (EV), wherein the EV comprises a single pass EV transmembrane protein fused to:
   (a) a moiety on the surface of the EV, and/or
   (b) a cargo molecule.
2. An EV comprising a single pass EV transmembrane protein fused to:
   (a) a moiety on the surface of the EV, and/or
   (b) a cargo molecule.
3. The composition and/or EV according to aspect 1 or 2, wherein the single pass EV transmembrane protein has a maximum molecular weight of 50 kDa.
4. The composition and/or EV according to any one of the previous aspects, wherein the single pass EV transmembrane protein comprises one or more post translational modification optionally selected from one or more of glycosylation such as N-linked GlcNAc asparagine, prenylation, ubiquitination, myristoylation, sumoylation, phosphorylation such as kinase specific phosphorylation, lipidation, hydroxyproline, pyrolidone and carboxylic acid.
5. The composition and/or EV according to any one of the previous aspects wherein the composition and/or EV is substantially devoid of vesicle aggregates; and/or the diameter of the EV is 30 to 150 nm or 150 to 1000 nm.
6. The composition and/or EV according to any one of the previous aspects, wherein the single pass EV transmembrane protein comprises:
   (a) a polypeptide sequence having at least 80%, at least 90%, at least 95% or at least 100% sequence identity to SEQ ID NO: 1
   (b) a polypeptide sequence having at least 80%, at least 90%, at least 95% or at least 100% sequence identity to any one of SEQ ID NOs: 2 to 5;
   (c) at least 10, at least 20, at least 30, at least 40 or at least 50 contiguous amino acid residues from the polypeptide sequence of any one of SEQ ID NOs: 1 to 5; and/or
   (d) pituitary tumour-transforming gene 1 interacting protein (PTTG1IP), or a variant or fragment thereof, optionally wherein the variant or fragment thereof retains the intrinsic cleavage activity of PTTG1IP.
6. The composition and/or EV according to any one of the previous aspects, wherein the EV comprises a further exogenous protein.
7. The composition and/or EV according to aspect 6, wherein the further exogenous protein is endosomal escape moiety, preferably VSVG.
7. The composition and/or EV according to any one of the previous aspects, wherein the moiety on the surface of the EV is selected from one or more of a peptide and/or protein, a targeting peptide and/or targeting protein which binds to a molecule present on a cell to be targeted, a therapeutic moiety such as a receptor decoy, an endosomal escape moiety, an enzyme, a nuclease, a CRISPR-associated protein (Cas) such as SaCas9, SpCas9, Cas9, Cas12, Cas13 and variants and fusions thereof, a Transcription activator-like effector nucleases (TALEN) and variants and fusions thereof, a meganuclease and variants and fusions thereof, a zinc finger nuclease and variants and fusions thereof, an antibody and/or antigen-binding variant or fragment thereof, a single chain variable fragment (scFv), VHH, a nanobody, a nucleic acid-binding protein and/or peptide, a RNA- and/or DNA-binding protein, viral-binding protein, a small molecule drug, a nucleic acid, a nucleic acid analogue, an unnatural nucleic acid, gRNA, miRNA, shRNA, siRNA, piRNA, PMO, DNA, a DNA plasmid encoding a therapeutic peptide and/or protein, and purification moiety.
8. The composition and/or EV according to aspect 7, wherein the moiety on the surface of the EV is a targeting peptide and/or targeting protein which binds to a molecule present on a cell to be targeted.
9. The composition and/or EV according to aspect 8, wherein the targeting peptide and/or targeting protein binds to a molecule present on a cell of the liver, preferably a hepatocyte; the heart, preferably a cardiomyocytes or a smooth muscle cell; the brain or the nervous system, preferable a neurone or a glial cell, most preferably a sensory neurone a motor neurone or an interneuron.
10. The composition and/or EV according to any one of aspects 7 to 9, wherein the moiety on the surface of the EV is selected from a peptide binding protein or an RNA binding protein.
11. The composition and/or EV according to aspect 10, wherein the peptide binding protein is capable of binding a Cas protein, preferably Cas9 or Cas12, or an adeno-associated virus (AAV), preferably an AAV capsid, and most preferably an AAV8 or AAV9 capsid.
12. The composition and/or EV according to any one of the previous aspects, wherein the cargo molecule is:
   (a) inside or outside the EV and/or
   (b) is an exogenous cargo molecule.
13. The composition and/or EV according to any one of the previous aspects, wherein the cargo molecule is selected from one or more of a therapeutic cargo, a peptide and/or protein, such as a therapeutic peptide and/or protein, an enzyme, a nuclease, a CRISPR-associated protein (Cas) such as SaCas9, SpCas9, Cas9, Cas12, Cas13 and variants and fusions thereof, a Transcription activator-like effector nucleases (TALEN) and variants and fusions thereof, a meganuclease and variants and fusions thereof, a zinc finger nuclease and variants and fusions thereof, an antibody and/or antigen-binding variant or fragment thereof, a single chain variable fragment (scFv), VHH, a nanobody, a binding protein for a therapeutic agent, such as an RNA-binding protein, a viral-binding protein, an Fc-binding protein or a small molecule-binding protein, a nucleic acid-binding protein and/or peptide, a RNA- and/or DNA-binding protein, a small molecule drug, a nucleic acid, a nucleic acid analogue, an unnatural nucleic acid, gRNA, miRNA, shRNA, siRNA, piRNA, PMO, DNA, a DNA plasmid encoding a therapeutic peptide and/or protein, and an endosomal escape moiety.
14. The composition and/or EV according to any one of the previous aspects, wherein the cargo molecule is a guide RNA, a Cas protein, preferably Cas9 or Cas12, or an adeno-associated virus (AAV).
15. The composition and/or EV according to aspect 13 or 14, wherein the therapeutic cargo is luminally loaded.
16. The composition and/or EV according to any one of the previous aspects, wherein the EV is exogenously loaded with the cargo molecule.
17. The composition and/or EV according to aspect 16, wherein the cargo molecule is loaded exogenously by electroporation, transfection reagent, co-incubation or by contact with a cell penetrating peptide (CPP) or any combination thereof.
18. The composition and/or EV according to aspect 17, wherein the cargo molecule is conjugated to a sterol and loaded by co-incubation.
19. The composition and/or EV according to any one of the previous aspects, wherein the composition and/or EV further comprises a release system, preferably wherein the release system comprises:
   (a) an organic compound-based release system;
   (b) a polypeptide-based release system;
   (c) a cis-cleaving polypeptide-based release system;
   (d) a cis-cleaving polypeptide-based release system comprising an intein;
   (e) a chemically induced dimerization system; and/or
   (f) a reversible protein-protein interaction module.
20. The composition and/or EV according to aspect 19, wherein the release system can be activated to release the cargo molecule from the EV.
21. The composition and/or EV according to any one or the previous aspects, wherein the composition and/or EV further comprises:
   (a) a linker; and/or
   (b) a multimerization domain.
22. The composition and/or EV according to any one of the previous aspects, wherein the composition and/or EV is co-administered with, and/or further comprises, an endosomal escape moiety that enhances release of the EV from endosomes.
23. The composition and/or EV according to aspect 22, wherein the molecule that enhances release of the EV from endosomes is:
   (a) a molecule that binds to protons;
   (b) a fusogenic protein; or
   (c) a cationic peptide or polymer.
24. The composition and/or EV according to aspect 22 or 23, wherein the molecule that enhances release of the EV from endosomes is:
   (a) an endosmolytic compound;
   (b) chloroquine, or a variant or derivative thereof, or
   (c) a cell penetrating peptide (CPP).
25. The composition and/or EV according to any one of the previous aspects, wherein the EV is an exosome.
26. The composition and/or EV according to any one of the previous aspects, wherein the EV is derived from HEK293 cells, HEK293T cells, adipocytes, and/or mesenchymal stem cells.
27. The composition and/or EV according to any one of the previous aspects for use in a method of delivering the cargo molecule in vitro, in vivo, and/or in situ.
28. A polypeptide construct comprising a single pass EV transmembrane protein fused to: (a) a moiety on the surface of the EV; and/or (b) a cargo molecule, according to any one of aspects 1 to 19.
29. A polynucleotide construct encoding the polypeptide construct according to aspect 28.
30. A cell comprising the polypeptide construct and/or the polynucleotide construct according to aspect 28 or 29.
31. A composition, EV, polypeptide construct, polynucleotide and/or cell according to any one of the previous aspects, and a pharmaceutically acceptable excipient.
32. The composition, EV, polypeptide construct, polynucleotide and/or cell according to any one of the previous aspects, and at least one pharmaceutically acceptable excipient, for use in a method of therapy in a subject, optionally wherein the subject is a human or animal subject.
33. The composition, EV, polypeptide construct, polynucleotide and/or cell, and at least one pharmaceutically acceptable excipient, for use in a method of treating and/or preventing Alzheimer's disease, autoimmune conditions, cancer, cardiovascular disease, cystic fibrosis, Duchenne muscular dystrophy, haemophilia, Huntington's disease, lysosomal storage diseases, liver disorders, macular degeneration, myotonic dystrophy, neuromuscular disease, Parkinson's disease, sepsis, spinal muscular atrophy or stroke.
34. The composition, EV, polypeptide construct, polynucleotide and/or cell, and at least one pharmaceutically acceptable excipient, for use in a method of treating or preventing genetic disorders, CNS conditions, neuro-degenerative disorders, liver disorders, heart disorders, Phenylketonuria, heart failure or ALS.
35. The composition, EV, polypeptide construct, polynucleotide and/or cell according to any one of aspects 32 to 34, wherein said use is gene therapy and/or gene editing.
36. A method of treatment comprising providing a composition, EV, polypeptide construct, polynucleotide and/or cell according to any one of aspects 1 to 35 and at least one pharmaceutically acceptable excipient to a subject in need thereof, optionally wherein the subject is a human or animal subject.
37. The method according to aspect 36, for treating and/or preventing Alzheimer's disease, autoimmune conditions, cancer, cardiovascular disease, cystic fibrosis, Duchenne muscular dystrophy, haemophilia, Huntington's disease, lysosomal storage disease, liver disorders, macular degeneration, myotonic dystrophy, neuromuscular disease, Parkinson's disease, sepsis, spinal muscular atrophy or stroke.
38. The method according to aspect 36, for treating and/or preventing genetic disorders, CNS conditions, neuro-degenerative disorders, liver disorders, heart disorders, Phenylketonuria, heart failure or ALS.
38. The method according to aspect 36 to 38, wherein said method comprises gene therapy and/or gene editing.
39. The use of a composition, EV, polypeptide construct, polynucleotide and/or cell according to any one of aspects 1 to 30 for purifying an EV.
40. The in vitro or ex vivo use of a composition, EV, polypeptide construct, polynucleotide and/or cell according to any one of aspects 1 to 30 as a research tool, a diagnostic tool, an imaging tool, a biological reference material, an experimental control and/or an experimental standard.
41. The use according to aspect 39 or 40, wherein the composition, EV, polypeptide construct, polynucleotide and/or cell is immobilised to a solid support.
42. A method of manufacturing a composition and/or EV according to any one of aspects 1 to 27 comprising:
   (a) introducing into a host cell a polynucleotide construct encoding the single pass EV transmembrane protein;
   (b) expressing the polynucleotide construct in the host cell; and
   (c) obtaining EVs in which the single pass EV transmembrane protein has been expressed.
43. The method according to aspect 42, wherein:
   (a) the polynucleotide construct encodes the single pass EV transmembrane protein fused to a moiety expressed on the surface of the EV and/or fused to a cargo molecule; and
   (b) expressing the polynucleotide construct in the host cell thereby generates an EV comprising a single pass EV transmembrane protein fused to a moiety on the surface of the EV and/or fused to a cargo molecule.
44. The method according to aspect 42 or 43, further comprising exogenously loading the composition and/or EV with the cargo molecule.
45. The method according to aspect 44, wherein the cargo molecule is loaded exogenously by electroporation, transfection reagent, co-incubation or by contact with a cell penetrating peptide (CPP) or any combination thereof.
46. The method according to any one or aspects 42 to 45, wherein the host cell is cultured in a bioreactor to produce a conditioned media and the EVs are obtained from the conditioned media.
47. The method according to aspect 46, wherein the EVs are purified from the conditioned media using one or more of liquid chromatography, tangential flow filtration, ultracentrifugation, density-gradient separation, antibody precipitation and polymer precipitation.

1. A composition comprising an extracellular vesicle (EV), wherein the EV comprises a single pass EV transmembrane protein fused to:
(a) a moiety on the surface of the EV, and/or
(b) a cargo molecule.

2. The composition according to claim 1, wherein the single pass EV transmembrane protein has a maximum molecular weight of 50 kDa.

3. The composition according to claim 1 or 2, wherein the single pass EV transmembrane protein comprises one or more post translational modification optionally selected from one or more of glycosylation such as N-linked GlcNAc asparagine, prenylation, ubiquitination, myristoylation, sumoylation, phosphorylation such as kinase specific phosphorylation, lipidation, hydroxyproline, pyrolidone and carboxylic acid.

4. The composition according to any one of the previous claims, wherein the composition is substantially devoid of vesicle aggregates; and/or the diameter of the EV is 30 to 150 nm or 150 to 1000 nm.

5. The composition according to any one of the previous claims, wherein the single pass EV transmembrane protein comprises:
(a) a polypeptide sequence having at least 80%, at least 90%, at least 95% or at least 100% sequence identity to SEQ ID NO: 1
(b) a polypeptide sequence having at least 80%, at least 90%, at least 95% or at least 100% sequence identity to any one of SEQ ID NOs: 2 to 5;
(c) at least 10, at least 20, at least 30, at least 40 or at least 50 contiguous amino acid residues from the polypeptide sequence of any one of SEQ ID NOs: 1 to 5; and/or
(d) pituitary tumour-transforming gene 1 interacting protein (PTTG1IP), or a variant or fragment thereof, optionally wherein the variant or fragment thereof retains the intrinsic cleavage activity of PTTG1IP;
preferably wherein the EV comprises a further exogenous protein, more preferably wherein the further exogenous protein is endosomal escape moiety, and most preferably wherein the endosomal escape moiety is VSVG.

6. The composition according to any one of the previous claims, wherein the moiety on the surface of the EV is selected from one or more of a peptide and/or protein, a targeting peptide and/or targeting protein which binds to a molecule present on a cell to be targeted, a therapeutic moiety such as a receptor decoy, an endosomal escape moiety, an enzyme, a nuclease, a CRISPR-associated protein (Cas) such as SaCas9, SpCas9, Cas9, Cas12, Cas13 and variants and fusions thereof, a Transcription activator-like effector nucleases (TALEN) and variants and fusions thereof, a meganuclease and variants and fusions thereof, a zinc finger nuclease and variants and fusions thereof, an antibody and/or antigen-binding variant or fragment thereof, a single chain variable fragment (scFv), VHH, a nanobody, a nucleic acid-binding protein and/or peptide, a RNA- and/or DNA-binding protein, viral-binding protein, a small molecule drug, a nucleic acid, a nucleic acid analogue, an unnatural nucleic acid, gRNA, miRNA, shRNA, siRNA, piRNA, PMO, DNA and a DNA plasmid encoding a therapeutic peptide and/or protein; optionally wherein:
(a) the moiety on the surface of the EV is a targeting peptide and/or targeting protein which binds to a molecule present on a cell to be targeted, optionally wherein the targeting peptide and/or targeting protein binds to a molecule present on a cell of the liver, preferably a hepatocyte; the heart, preferably a cardiomyocytes or a smooth muscle cell; the brain or the nervous system, preferable a neurone or a glial cell, most preferably a sensory neurone a motor neurone or an interneuron; and/or
(b) the moiety on the surface of the EV is selected from a peptide binding protein or an RNA binding protein, optionally wherein the peptide binding protein is capable of binding a Cas protein, preferably Cas9 or Cas12, or an adeno-associated virus (AAV), preferably an AAV capsid, and most preferably an AAV8 or AAV9 capsid.

7. The composition according to any one of the previous claims, wherein the cargo molecule is:
(a) inside or outside the EV and/or
(b) is an exogenous cargo molecule.

8. The composition according to any one of the previous claims, wherein the cargo molecule is selected from one or more of a therapeutic cargo, a peptide and/or protein, an enzyme, a nuclease, a CRISPR-associated protein (Cas) such as SaCas9, SpCas9, Cas9, Cas12, Cas13 and variants and fusions thereof, a Transcription activator-like effector nucleases (TALEN) and variants and fusions thereof, a meganuclease and variants and fusions thereof, a zinc finger nuclease and variants and fusions thereof, an antibody and/or antigen-binding variant or fragment thereof, a single chain variable fragment (scFv), VHH, a nanobody, a nucleic acid-binding protein and/or peptide, a RNA- and/or DNA-binding protein, viral-binding protein, a small molecule drug, a nucleic acid, a nucleic acid analogue, an unnatural nucleic acid, gRNA, miRNA, shRNA, siRNA, piRNA, PMO, DNA and a DNA plasmid encoding a therapeutic peptide and/or protein;
optionally wherein the cargo molecule is a guide RNA, a Cas protein, preferably Cas9 or Cas12, or an adeno-associated virus (AAV) and/or wherein the therapeutic cargo is luminally loaded.

9. The composition according to any one of the previous claims, wherein the composition further comprises a release system, preferably wherein the release system comprises:
(a) an organic compound-based release system;
(b) a polypeptide-based release system;
(c) a cis-cleaving polypeptide-based release system;
(d) a cis-cleaving polypeptide-based release system comprising an intein;
(e) a chemically induced dimerization system; and/or
(f) a reversible protein-protein interaction module.

10. The composition according to claim 9, wherein the release system can be activated to release the cargo molecule from the EV.

11. The composition according to any one of the previous claims, wherein the composition is co-administered with, and/or further comprises, an endosomal escape moiety that enhances release of the EV from endosomes.

12. The composition according to claim 11, wherein the molecule that enhances release of the EV from endosomes is:
(a) a molecule that binds to protons;
(b) a fusogenic protein; or
(c) a cationic peptide or polymer.

13. The composition according to claim 11 or 12, wherein the molecule that enhances release of the EV from endosomes is:
(a) an endosmolytic compound;
(b) chloroquine, or a variant or derivative thereof, or
(c) a cell penetrating peptide (CPP).

14. The composition according to any one of the previous claims, wherein the EV is an exosome.

15. The composition according to any one of the previous claims, wherein the EV is derived from HEK293 cells, HEK293T cells, adipocytes, and/or mesenchymal stem cells.

16. The composition according to any one of the previous claims for use in a method of delivering the cargo molecule in vitro, in vivo, and/or in situ.

17. The composition according to any one of the previous claims and at least one pharmaceutically acceptable excipient, for use in a method of therapy in a subject.

18. The composition according to any one of the previous claims and at least one pharmaceutically acceptable excipient, for use in a method of treating and/or preventing Alzheimer's disease, autoimmune conditions, cancer, cardiovascular disease, cystic fibrosis, Duchenne muscular dystrophy, haemophilia, Huntington's disease, lysosomal storage disease, liver disorders, macular degeneration, myotonic dystrophy, neuromuscular disease, Parkinson's disease, sepsis, spinal muscular atrophy, stroke, genetic disorders, CNS conditions, neuro-degenerative disorders, heart disorders, Phenylketonuria, heart failure or ALS.

19. The composition according to claims 17 or 18, wherein said use is gene therapy and/or gene editing.

20. The use of a composition according to any one of claims 1 to 15 for purifying an EV.

21. The in vitro or ex vivo use of a composition according to any one of claims 1 to 15 as a research tool, a diagnostic tool, an imaging tool, a biological reference material, an experimental control and/or an experimental standard.

22. The use according to claim 20 or 21, wherein the composition is immobilised to a solid support.

23. A method of manufacturing a composition according to any one of claims 1 to 15 comprising:
(a) introducing into a host cell a polynucleotide construct encoding the single pass EV transmembrane protein;
(b) expressing the polynucleotide construct in the host cell; and
(c) obtaining EVs in which the single pass EV transmembrane protein has been expressed.

24. The method according to claim 23, wherein the host cell is cultured in a bioreactor to produce a conditioned media and the EVs are obtained from the conditioned media;
optionally wherein the EVs are purified from the conditioned media using one or more of liquid chromatography, tangential flow filtration, ultracentrifugation, density-gradient separation, antibody precipitation and polymer precipitation.

25. A polypeptide construct comprising a single pass EV transmembrane protein fused to: (a) a moiety on the surface of the EV; and/or (b) a cargo molecule, according to any one of claims 1 to 10; a polynucleotide construct encoding the polypeptide construct; or a cell comprising the polypeptide construct and/or the polynucleotide construct.
